SEQUENCE LISTING (300 sequences)

SEQ ID NO: 1 - protein, 14 aa, synthetic construct
GLTSGSVSTSHYPS

SEQ ID NO: 2 - protein, 7 aa, synthetic construct
RTNTRSS

SEQ ID NO: 3 - protein, 10 aa, synthetic construct
VLFMGSGTWV

SEQ ID NO: 4 - protein, 10 aa, synthetic construct
GFTVSRSYMS

SEQ ID NO: 5 - protein, 16 aa, synthetic construct
IIYRDGTTYYADSVKG

SEQ ID NO: 6 - protein, 8 aa, synthetic construct
EVDYAFDP

SEQ ID NO: 7 - protein, 14 aa, synthetic construct
GLTSGSVSTSHYPS

SEQ ID NO: 8 - protein, 7 aa, synthetic construct
RTNTRSS

SEQ ID NO: 9 - protein, 10 aa, synthetic construct
VLFMGSGTWV

SEQ ID NO: 10 - protein, 10 aa, synthetic construct
GFTVSRSYMS

SEQ ID NO: 11 - protein, 16 aa, synthetic construct
IIYRDGTTYYADSVKG

SEQ ID NO: 12 - protein, 8 aa, synthetic construct
EVDYAFDP

SEQ ID NO: 13 - protein, 110 aa, synthetic construct
QTVVTQEPSFSVSPGGTVTLTCGLTSGSVSTSHYPSWYQQTPGQAPRTLIYRTNTRSSGVPDRFSGSILGNKAALTITGAQADDESDYYCVLFMGSGTWVFGGGTKLTVL

SEQ ID NO: 14 - protein, 116 aa, synthetic construct
EVQLVESGGGLIQPGGSLRLSCAASGFTVSRSYMSWVRQAPGKGLEWVSIIYRDGTTYYADSVKGRFTISRDNSKNTLYLQMNSLRADDTAVYYCAREVDYAFDPWGQGTLVTVSS

SEQ ID NO: 15 - protein, 110 aa, synthetic construct; identical to SEQ ID NO: 13

SEQ ID NO: 16 - protein, 116 aa, synthetic construct; identical to SEQ ID NO: 14

SEQ ID NO: 17 - protein, 216 aa, synthetic construct
QTVVTQEPSFSVSPGGTVTLTCGLTSGSVSTSHYPSWYQQTPGQAPRTLIYRTNTRSSGVPDRFSGSILGNKAALTITGAQADDESDYYCVLFMGSGTWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 18 - protein, 446 aa, synthetic construct
EVQLVESGGGLIQPGGSLRLSCAASGFTVSRSYMSWVRQAPGKGLEWVSIIYRDGTTYYADSVKGRFTISRDNSKNTLYLQMNSLRADDTAVYYCAREVDYAFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 216 aa, synthetic construct
QTVVTQEPSFSVSPGGTVTLTCGLTSGSVSTSHYPSWYQQTPGQAPRTLIYRTNTRSSGVPDRFSGSILGNKAALTITGAQADDESDYYCVLFMGSGTWVFGGGTKLTVLGQPAVTPSVILFPPSSEELKDNKATLVCLISDFYPRTVKVNWKADGNSVTQGVDTTQPSKQSNNKYAASSFLHLTANQWKSYQSVTCQVTHEGHTVEKSLAPAECS

SEQ ID NO: 20 - protein, 439 aa, synthetic construct
EVQLVESGGGLIQPGGSLRLSCAASGFTVSRSYMSWVRQAPGKGLEWVSIIYRDGTTYYADSVKGRFTISRDNSKNTLYLQMNSLRADDTAVYYCAREVDYAFDPWGQGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 21 - protein, 11 aa, synthetic construct
QASQSIGSSLA

SEQ ID NO: 22 - protein, 7 aa, synthetic construct
QASKLAS

SEQ ID NO: 23 - protein, 12 aa, synthetic construct
QCTGNGGDFIGA

SEQ ID NO: 24 - protein, 10 aa, synthetic construct
GFSLSSYYVR

SEQ ID NO: 25 - protein, 16 aa, synthetic construct
IISDSGSTYYASWAKG

SEQ ID NO: 26 - protein, 10 aa, synthetic construct
GSGYTIPTDL

SEQ ID NO: 27 - protein, 11 aa, synthetic construct
QASQSIGSSLA

SEQ ID NO: 28 - protein, 7 aa, synthetic construct
QASKLAS

SEQ ID NO: 29 - protein, 12 aa, synthetic construct
QCTGNGGDFIGA

SEQ ID NO: 30 - protein, 10 aa, synthetic construct
GFSLSSYYVR

SEQ ID NO: 31 - protein, 16 aa, synthetic construct
IISDSGSTYYASWAKG

SEQ ID NO: 32 - protein, 10 aa, synthetic construct
GSGYTIPTDL

SEQ ID NO: 33 - protein, 112 aa, synthetic construct
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPYTFGGGTKLEIK

SEQ ID NO: 34 - protein, 118 aa, synthetic construct
QVQLKQSGPGLVQPSQSLSITCTVSGFSLTSYGVHWVRQSPGKGLEWLGVIWSGGSTDYNAAFISRLSISKDNSKSQVFFKMNSLQANDTAIYYCARNDYDRPIFAYWGQGTLVTVSA

SEQ ID NO: 35 - protein, 110 aa, synthetic construct
DPVMTQTPASVSEPVGGTVTIKCQASQSIGSSLAWYQQKPGQRPKLLIYQASKLASGVPSRFKGSRSGTEFTLTISDLECADAATYYCQCTGNGGDFIGAFGGGTEVVVK

SEQ ID NO: 36 - protein, 115 aa, synthetic construct
QSLEESGGGLVTPGTPLTLTCTASGFSLSSYYVRWVRQAPGKGLEYIGIISDSGSTYYASWAKGRFTISKTSTTVDLKFTSPTTEDTATYFCARGSGYTIPTDLWGPGTLVTVSS

SEQ ID NO: 37 - protein, 219 aa, synthetic construct
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 448 aa, synthetic construct
QVQLKQSGPGLVQPSQSLSITCTVSGFSLTSYGVHWVRQSPGKGLEWLGVIWSGGSTDYNAAFISRLSISKDNSKSQVFFKMNSLQANDTAIYYCARNDYDRPIFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 214 aa, synthetic construct
DPVMTQTPASVSEPVGGTVTIKCQASQSIGSSLAWYQQKPGQRPKLLIYQASKLASGVPSRFKGSRSGTEFTLTISDLECADAATYYCQCTGNGGDFIGAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 40 - protein, 438 aa, synthetic construct
QSLEESGGGLVTPGTPLTLTCTASGFSLSSYYVRWVRQAPGKGLEYIGIISDSGSTYYASWAKGRFTISKTSTTVDLKFTSPTTEDTATYFCARGSGYTIPTDLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 41 - protein, 17 aa, synthetic construct
KSSQSLLYSSNQRTYLA

SEQ ID NO: 42 - protein, 7 aa, synthetic construct
WVSTREF

SEQ ID NO: 43 - protein, 8 aa, synthetic construct
HQYLSMFT

SEQ ID NO: 44 - protein, 10 aa, synthetic construct
GFTFSSFGMH

SEQ ID NO: 45 - protein, 16 aa, synthetic construct
YISSDNAIYYADTVEG

SEQ ID NO: 46 - protein, 7 aa, synthetic construct
SAWYFNV

SEQ ID NO: 47 - protein, 17 aa, synthetic construct
KSSQSLLYSSNQRTYLA

SEQ ID NO: 48 - protein, 7 aa, synthetic construct
WVSTREF

SEQ ID NO: 49 - protein, 8 aa, synthetic construct
HQYLSMFT

SEQ ID NO: 50 - protein, 10 aa, synthetic construct
GFTFSSFGMH

SEQ ID NO: 51 - protein, 16 aa, synthetic construct
YISSDNAIYYADTVEG

SEQ ID NO: 52 - protein, 7 aa, synthetic construct
SAWYFNV

SEQ ID NO: 53 - protein, 112 aa, synthetic construct
KIIMTQSPSSLAVSAGEEVTMTCKSSQSLLYSSNQRTYLAWYQQKPGQSPKLLIYWVSTREFGVPDRFTGSGSGTDFTLTISSVQPEDLAVYYCHQYLSMFTFGSGTRLELK

SEQ ID NO: 54 - protein, 115 aa, synthetic construct
DVQLVESGGGLVQPGGSRKLSCAASGFTFSSFGMHWVRQAPEKGLEWVAYISSDNAIYYADTVEGRFTISRDNPKNTLFLQMTSLRSEDTAIYYCARSAWYFNVWGAGTTVTVSS

SEQ ID NO: 55 - protein, 112 aa, synthetic construct; identical to SEQ ID NO: 53

SEQ ID NO: 56 - protein, 115 aa, synthetic construct; identical to SEQ ID NO: 54

SEQ ID NO: 57 - protein, 219 aa, synthetic construct
KIIMTQSPSSLAVSAGEEVTMTCKSSQSLLYSSNQRTYLAWYQQKPGQSPKLLIYWVSTREFGVPDRFTGSGSGTDFTLTISSVQPEDLAVYYCHQYLSMFTFGSGTRLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 58 - protein, 439 aa, synthetic construct
DVQLVESGGGLVQPGGSRKLSCAASGFTFSSFGMHWVRQAPEKGLEWVAYISSDNAIYYADTVEGRFTISRDNPKNTLFLQMTSLRSEDTAIYYCARSAWYFNVWGAGTTVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 59 - protein, 216 aa, synthetic construct
KIIMTQSPSSLAVSAGEEVTMTCKSSQSLLYSSNQRTYLAWYQQKPGQSPKLLIYWVSTREFGVPDRFTGSGSGTDFTLTISSVQPEDLAVYYCHQYLSMFTFGSGTRLELKRDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 60 - protein, 438 aa, synthetic construct
DVQLVESGGGLVQPGGSRKLSCAASGFTFSSFGMHWVRQAPEKGLEWVAYISSDNAIYYADTVEGRFTISRDNPKNTLFLQMTSLRSEDTAIYYCARSAWYFNVWGAGTTVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSEDDPEVQFTWYINNEQVRTARPPLREQQFNSTIRVVSTLPIAHEDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 61 - protein, 11 aa, synthetic construct
QASQNTDIRLA

SEQ ID NO: 62 - protein, 7 aa, synthetic construct
SASTLAS

SEQ ID NO: 63 - protein, 13 aa, synthetic construct
LGNYDCSYADCYA

SEQ ID NO: 64 - protein, 10 aa, synthetic construct
GIDLNNYEMG

SEQ ID NO: 65 - protein, 16 aa, synthetic construct
YIRTGGSAYYASWAKG

SEQ ID NO: 66 - protein, 11 aa, synthetic construct
PYAFVSLINDL

SEQ ID NO: 67 - protein, 11 aa, synthetic construct
QASQNTDIRLA

SEQ ID NO: 68 - protein, 7 aa, synthetic construct
SASTLAS

SEQ ID NO: 69 - protein, 13 aa, synthetic construct
LGNYDCSYADCYA

SEQ ID NO: 70 - protein, 10 aa, synthetic construct
GIDLNNYEMG

SEQ ID NO: 71 - protein, 16 aa, synthetic construct
YIRTGGSAYYASWAKG

SEQ ID NO: 72 - protein, 11 aa, synthetic construct
PYAFVSLINDL

SEQ ID NO: 73 - protein, 111 aa, synthetic construct
AQVLTQTPSSVSAAVGGTVTINCQASQNTDIRLAWYQQKPGQPPKRLIYSASTLASGVPSRFKGSGSGTQFTLTISDLECDDAATYYCLGNYDCSYADCYAFGGGTEVVVR

SEQ ID NO: 74 - protein, 116 aa, synthetic construct
QSLEESGGRLVTPGTPLTLTCTVSGIDLNNYEMGWVRQAPGKGLEWIGYIRTGGSAYYASWAKGRFTISKTSTTVDLKMTSLATEDTATYFCARPYAFVSLINDLWGPGTLVTVSS

SEQ ID NO: 75 - protein, 111 aa, synthetic construct; identical to SEQ ID NO: 73

SEQ ID NO: 76 - protein, 116 aa, synthetic construct; identical to SEQ ID NO: 74

SEQ ID NO: 77 - protein, 218 aa, synthetic construct
AQVLTQTPSSVSAAVGGTVTINCQASQNTDIRLAWYQQKPGQPPKRLIYSASTLASGVPSRFKGSGSGTQFTLTISDLECDDAATYYCLGNYDCSYADCYAFGGGTEVVVRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 446 aa, synthetic construct
QSLEESGGRLVTPGTPLTLTCTVSGIDLNNYEMGWVRQAPGKGLEWIGYIRTGGSAYYASWAKGRFTISKTSTTVDLKMTSLATEDTATYFCARPYAFVSLINDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 215 aa, synthetic construct
AQVLTQTPSSVSAAVGGTVTINCQASQNTDIRLAWYQQKPGQPPKRLIYSASTLASGVPSRFKGSGSGTQFTLTISDLECDDAATYYCLGNYDCSYADCYAFGGGTEVVVRGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 80 - protein, 439 aa, synthetic construct
QSLEESGGRLVTPGTPLTLTCTVSGIDLNNYEMGWVRQAPGKGLEWIGYIRTGGSAYYASWAKGRFTISKTSTTVDLKMTSLATEDTATYFCARPYAFVSLINDLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 81 - protein, 13 aa, synthetic construct
QASQSVWKNNYLS

SEQ ID NO: 82 - protein, 7 aa, synthetic construct
TASSLAS

SEQ ID NO: 83 - protein, 10 aa, synthetic construct
AGDVGGGIRT

SEQ ID NO: 84 - protein, 10 aa, synthetic construct
GFSLSSYTTS

SEQ ID NO: 85 - protein, 17 aa, synthetic construct
VIDVGSDDTYYATWAKG

SEQ ID NO: 86 - protein, 11 aa, synthetic construct
HGATGGTVFDL

SEQ ID NO: 87 - protein, 13 aa, synthetic construct
QASQSVWKNNYLS

SEQ ID NO: 88 - protein, 7 aa, synthetic construct
TASSLAS

SEQ ID NO: 89 - protein, 10 aa, synthetic construct
AGDVGGGIRT

SEQ ID NO: 90 - protein, 10 aa, synthetic construct
GFSLSSYTTS

SEQ ID NO: 91 - protein, 16 aa, synthetic construct
VIDVGSDDTYYATWAK

SEQ ID NO: 92 - protein, 11 aa, synthetic construct
HGATGGTVFDL

SEQ ID NO: 93 - protein, 110 aa, synthetic construct
AQVLTQTPSPVSAAVGGTVTINCQASQSVWKNNYLSWFQQKPGQPPKLLIYTASSLASGVPSRFKGSGSGTQFTLTISDLECDDAATYYCAGDVGGGIRTFGGGTEVVVK

SEQ ID NO: 94 - protein, 117 aa, synthetic construct
QSLEESGGRLVTPGTPLTLTCTASGFSLSSYTTSWVRQAPGKGLEWVGVIDVGSDDTYYATWAKGRFTISRTSTTVDLKMTSLTAADTATYFCTRHGATGGTVFDLWGPGTLVTVSS

SEQ ID NO: 95 - protein, 109 aa, synthetic construct
AQVLTQTPSPVSAAVGGTVTINCQASQSVWKNNYLSWFQQKPGQPPKLLIYTASSLASGVPSRFKGSGSGQFTLTISDLECDDAATYYCAGDVGGGIRTFGGGTEVVVK

SEQ ID NO: 96 - protein, 117 aa, synthetic construct; identical to SEQ ID NO: 94

SEQ ID NO: 97 - protein, 217 aa, synthetic construct
AQVLTQTPSPVSAAVGGTVTINCQASQSVWKNNYLSWFQQKPGQPPKLLIYTASSLASGVPSRFKGSGSGTQFTLTISDLECDDAATYYCAGDVGGGIRTFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 98 - protein, 447 aa, synthetic construct
QSLEESGGRLVTPGTPLTLTCTASGFSLSSYTTSWVRQAPGKGLEWVGVIDVGSDDTYYATWAKGRFTISRTSTTVDLKMTSLTAADTATYFCTRHGATGGTVFDLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 214 aa, synthetic construct
AQVLTQTPSPVSAAVGGTVTINCQASQSVWKNNYLSWFQQKPGQPPKLLIYTASSLASGVPSRFKGSGSGTQFTLTISDLECDDAATYYCAGDVGGGIRTFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 100 - protein, 440 aa, synthetic construct
QSLEESGGRLVTPGTPLTLTCTASGFSLSSYTTSWVRQAPGKGLEWVGVIDVGSDDTYYATWAKGRFTISRTSTTVDLKMTSLTAADTATYFCTRHGATGGTVFDLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 101 - protein, 17 aa, synthetic construct
KSSQSVLYSSNQKNYLA

SEQ ID NO: 102 - protein, 7 aa, synthetic construct
WASTRES

SEQ ID NO: 103 - protein, 8 aa, synthetic construct
HQHLSSWT

SEQ ID NO: 104 - protein, 10 aa, synthetic construct
GFTFRNYGMS

SEQ ID NO: 105 - protein, 17 aa, synthetic construct
VINSNGGKTYYPDSVKG

SEQ ID NO: 106 - protein, 8 aa, synthetic construct
DGKGWFAY

SEQ ID NO: 107 - protein, 17 aa, synthetic construct
KSSQSVLYSSNQKNYLA

SEQ ID NO: 108 - protein, 7 aa, synthetic construct
WASTRES

SEQ ID NO: 109 - protein, 8 aa, synthetic construct
HQHLSSWT

SEQ ID NO: 110 - protein, 10 aa, synthetic construct
GFTFRNYGMS

SEQ ID NO: 111 - protein, 17 aa, synthetic construct
VINSNGGKTYYPDSVKG

SEQ ID NO: 112 - protein, 8 aa, synthetic construct
DGKGWFAY

SEQ ID NO: 113 - protein, 112 aa, synthetic construct
NIMMTQSPSSLAVSAGEKVTMNCKSSQSVLYSSNQKNYLAWYQQKPGQSPKLLIYWASTRESGVPDRFTGSGSGTDFALTISSVQLEDLAVYYCHQHLSSWTFGGGTKVEIK

SEQ ID NO: 114 - protein, 117 aa, synthetic construct
EVQLVESGGGLVQPGGSLKLSCAASGFTFRNYGMSWVRQTPDKRLELVAVINSNGGKTYYPDSVKGRFTISRDNAKNTLYLQMSSLKSEDTAMYYCARDGKGWFAYWGQGTLVTVSA

SEQ ID NO: 115 - protein, 112 aa, synthetic construct; identical to SEQ ID NO: 113

SEQ ID NO: 116 - protein, 117 aa, synthetic construct; identical to SEQ ID NO: 114

SEQ ID NO: 117 - protein, 219 aa, synthetic construct
NIMMTQSPSSLAVSAGEKVTMNCKSSQSVLYSSNQKNYLAWYQQKPGQSPKLLIYWASTRESGVPDRFTGSGSGTDFALTISSVQLEDLAVYYCHQHLSSWTFGGGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 118 - protein, 441 aa, synthetic construct
EVQLVESGGGLVQPGGSLKLSCAASGFTFRNYGMSWVRQTPDKRLELVAVINSNGGKTYYPDSVKGRFTISRDNAKNTLYLQMSSLKSEDTAMYYCARDGKGWFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 119 - protein, 216 aa, synthetic construct
NIMMTQSPSSLAVSAGEKVTMNCKSSQSVLYSSNQKNYLAWYQQKPGQSPKLLIYWASTRESGVPDRFTGSGSGTDFALTISSVQLEDLAVYYCHQHLSSWTFGGGTKVEIKRDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 120 - protein, 440 aa, synthetic construct
EVQLVESGGGLVQPGGSLKLSCAASGFTFRNYGMSWVRQTPDKRLELVAVINSNGGKTYYPDSVKGRFTISRDNAKNTLYLQMSSLKSEDTAMYYCARDGKGWFAYWGQGTLVTVSAGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSEDDPEVQFTWYINNEQVRTARPPLREQQFNSTIRVVSTLPIAHEDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 121 - protein, 13 aa, synthetic construct
TGNSNNVGNQGAA

SEQ ID NO: 122 - protein, 7 aa, synthetic construct
RNNNRPS

SEQ ID NO: 123 - protein, 11 aa, synthetic construct
SAWDSSLNAWV

SEQ ID NO: 124 - protein, 10 aa, synthetic construct
GFTFSNYVMH

SEQ ID NO: 125 - protein, 17 aa, synthetic construct
LIWSDGSTIFHADSVKG

SEQ ID NO: 126 - protein, 14 aa, synthetic construct
EPRAIADNYYGMDV

SEQ ID NO: 127 - protein, 13 aa, synthetic construct
TGNSNNVGNQGAA

SEQ ID NO: 128 - protein, 7 aa, synthetic construct
RNNNRPS

SEQ ID NO: 129 - protein, 11 aa, synthetic construct
SAWDSSLNAWV

SEQ ID NO: 130 - protein, 10 aa, synthetic construct
GFTFSNYVMH

SEQ ID NO: 131 - protein, 17 aa, synthetic construct
LIWSDGSTIFHADSVKG

SEQ ID NO: 132 - protein, 14 aa, synthetic construct
EPRAIADNYYGMDV

SEQ ID NO: 133 - protein, 110 aa, synthetic construct
QAGLTQPPSVSKGLRQTATLTCTGNSNNVGNQGAAWLQQHQGHPPKLLSYRNNNRPSGISERLSASRSGNTASLTITGLQPEDEADYYCSAWDSSLNAWVFGGGTKLAVL

SEQ ID NO: 134 - protein, 123 aa, synthetic construct
QVQLVESGGDVVQPGRSLRLSCAASGFTFSNYVMHWVRQAPGEGLEWVSLIWSDGSTIFHADSVKGRFTISRDNSKNTLYLQMDSLRAEDTAVYYCAREPRAIADNYYGMDVWGQGTSVTVSS

SEQ ID NO: 135 - protein, 110 aa, synthetic construct; identical to SEQ ID NO: 133

SEQ ID NO: 136 - protein, 123 aa, synthetic construct; identical to SEQ ID NO: 134

SEQ ID NO: 137 - protein, 216 aa, synthetic construct
QAGLTQPPSVSKGLRQTATLTCTGNSNNVGNQGAAWLQQHQGHPPKLLSYRNNNRPSGISERLSASRSGNTASLTITGLQPEDEADYYCSAWDSSLNAWVFGGGTKLAVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 138 - protein, 453 aa, synthetic construct
QVQLVESGGDVVQPGRSLRLSCAASGFTFSNYVMHWVRQAPGEGLEWVSLIWSDGSTIFHADSVKGRFTISRDNSKNTLYLQMDSLRAEDTAVYYCAREPRAIADNYYGMDVWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - protein, 216 aa, synthetic construct
QAGLTQPPSVSKGLRQTATLTCTGNSNNVGNQGAAWLQQHQGHPPKLLSYRNNNRPSGISERLSASRSGNTASLTITGLQPEDEADYYCSAWDSSLNAWVFGGGTKLAVLGQPAVTPSVILFPPSSEELKDNKATLVCLISDFYPRTVKVNWKADGNSVTQGVDTTQPSKQSNNKYAASSFLHLTANQWKSYQSVTCQVTHEGHTVEKSLAPAECS

SEQ ID NO: 140 - protein, 446 aa, synthetic construct
QVQLVESGGDVVQPGRSLRLSCAASGFTFSNYVMHWVRQAPGEGLEWVSLIWSDGSTIFHADSVKGRFTISRDNSKNTLYLQMDSLRAEDTAVYYCAREPRAIADNYYGMDVWGQGTSVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 141 - protein, 13 aa, synthetic construct
QASKTVYDDNALA

SEQ ID NO: 142 - protein, 7 aa, synthetic construct
KASTLAS

SEQ ID NO: 143 - protein, 12 aa, synthetic construct
AGGYIYDSGDHA

SEQ ID NO: 144 - protein, 10 aa, synthetic construct
GIDLSRGQVG

SEQ ID NO: 145 - protein, 16 aa, synthetic construct
FKGYGGNAFYTNWAKG

SEQ ID NO: 146 - protein, 10 aa, synthetic construct
VAGDINHLDL

SEQ ID NO: 147 - protein, 13 aa, synthetic construct
QASKTVYDDNALA

SEQ ID NO: 148 - protein, 7 aa, synthetic construct
KASTLAS

SEQ ID NO: 149 - protein, 12 aa, synthetic construct
AGGYIYDSGDHA

SEQ ID NO: 150 - protein, 10 aa, synthetic construct
GIDLSRGQVG

SEQ ID NO: 151 - protein, 16 aa, synthetic construct
FKGYGGNAFYTNWAKG

SEQ ID NO: 152 - protein, 10 aa, synthetic construct
VAGDINHLDL

SEQ ID NO: 153 - protein, 112 aa, synthetic construct
AAVLTQTPSPVSAAVGGTVSINCQASKTVYDDNALAWYQQKPGQPPKLLIYKASTLASGVPSRFSGSGSGTQFTLTISDVQCDDAATYYCAGGYIYDSGDHAFGGGTEVVVK

SEQ ID NO: 154 - protein, 116 aa, synthetic construct
QSLEESGGRLVTPGTPLTLTCTVSGIDLSRGQVGWVRQAPGEGLEYIGFKGYGGNAFYTNWAKGRFTISKTSSTTVDLKITTPTTEDTATYFCARVAGDINHLDLWGQGTLVTVSS

SEQ ID NO: 155 - protein, 112 aa, synthetic construct; identical to SEQ ID NO: 153

SEQ ID NO: 156 - protein, 116 aa, synthetic construct; identical to SEQ ID NO: 154

SEQ ID NO: 157 - protein, 219 aa, synthetic construct
AAVLTQTPSPVSAAVGGTVSINCQASKTVYDDNALAWYQQKPGQPPKLLIYKASTLASGVPSRFSGSGSGTQFTLTISDVQCDDAATYYCAGGYIYDSGDHAFGGGTEVVVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 158 - protein, 440 aa, synthetic construct
QSLEESGGRLVTPGTPLTLTCTVSGIDLSRGQVGWVRQAPGEGLEYIGFKGYGGNAFYTNWAKGRFTISKTSSTTVDLKITTPTTEDTATYFCARVAGDINHLDLWGQGTLVTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 159 - protein, 216 aa, synthetic construct
AAVLTQTPSPVSAAVGGTVSINCQASKTVYDDNALAWYQQKPGQPPKLLIYKASTLASGVPSRFSGSGSGTQFTLTISDVQCDDAATYYCAGGYIYDSGDHAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 160 - protein, 439 aa, synthetic construct
QSLEESGGRLVTPGTPLTLTCTVSGIDLSRGQVGWVRQAPGEGLEYIGFKGYGGNAFYTNWAKGRFTISKTSSTTVDLKITTPTTEDTATYFCARVAGDINHLDLWGQGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 161 - protein, 16 aa, synthetic construct
RSSQSIVYSDGNTYLE

SEQ ID NO: 162 - protein, 7 aa, synthetic construct
KVSHRFS

SEQ ID NO: 163 - protein, 9 aa, synthetic construct
FQGSHVPWT

SEQ ID NO: 164 - protein, 10 aa, synthetic construct
GYTFTDYYMK

SEQ ID NO: 165 - protein, 17 aa, synthetic construct
DINPNNYDTNYNQKFKG

SEQ ID NO: 166 - skipped

SEQ ID NO: 167 - protein, 16 aa, synthetic construct
RSSQSIVYSDGNTYLE

SEQ ID NO: 168 - protein, 7 aa, synthetic construct
KVSHRFS

SEQ ID NO: 169 - protein, 9 aa, synthetic construct
FQGSHVPWT

SEQ ID NO: 170 - protein, 10 aa, synthetic construct
GYTFTDYYMK

SEQ ID NO: 171 - protein, 17 aa, synthetic construct
DINPNNYDTNYNQKFKG

SEQ ID NO: 172 - skipped

SEQ ID NO: 173 - protein, 112 aa, synthetic construct
DVLMTQTPLSLPVSLGDQASIFCRSSQSIVYSDGNTYLEWYLQKPGQSPKLLIYKVSHRFSGVPDRFSGSGSGTDFTLKISRVEAEDLGVYFCFQGSHVPWTFGGGTKLEIK

SEQ ID NO: 174 - protein, 112 aa, synthetic construct
EVQLQQSGPGLVKPGASVKMSCKASGYTFTDYYMKWMKQSHGKSLEWIGDINPNNYDTNYNQKFKGRATLTVDKSSSTAYMQLNSLTSEDSAVYYCARWDYWGQGTTLTVSS

SEQ ID NO: 175 - protein, 112 aa, synthetic construct; identical to SEQ ID NO: 173

SEQ ID NO: 176 - protein, 112 aa, synthetic construct; identical to SEQ ID NO: 174

SEQ ID NO: 177 - protein, 219 aa, synthetic construct
DVLMTQTPLSLPVSLGDQASIFCRSSQSIVYSDGNTYLEWYLQKPGQSPKLLIYKVSHRFSGVPDRFSGSGSGTDFTLKISRVEAEDLGVYFCFQGSHVPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 178 - protein, 436 aa, synthetic construct
EVQLQQSGPGLVKPGASVKMSCKASGYTFTDYYMKWMKQSHGKSLEWIGDINPNNYDTNYNQKFKGRATLTVDKSSSTAYMQLNSLTSEDSAVYYCARWDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 179 - protein, 216 aa, synthetic construct
DVLMTQTPLSLPVSLGDQASIFCRSSQSIVYSDGNTYLEWYLQKPGQSPKLLIYKVSHRFSGVPDRFSGSGSGTDFTLKISRVEAEDLGVYFCFQGSHVPWTFGGGTKLEIKRDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 180 - protein, 435 aa, synthetic construct
EVQLQQSGPGLVKPGASVKMSCKASGYTFTDYYMKWMKQSHGKSLEWIGDINPNNYDTNYNQKFKGRATLTVDKSSSTAYMQLNSLTSEDSAVYYCARWDYWGQGTTLTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSEDDPEVQFTWYINNEQVRTARPPLREQQFNSTIRVVSTLPIAHEDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 181 - protein, 11 aa, synthetic construct
RASQSIDNYLN

SEQ ID NO: 182 - protein, 7 aa, synthetic construct
AASSLQS

SEQ ID NO: 183 - protein, 9 aa, synthetic construct
QQSYSSPYT

SEQ ID NO: 184 - protein, 10 aa, synthetic construct
GLTFSNSWMS

SEQ ID NO: 185 - protein, 17 aa, synthetic construct
NINQDAGQKYSVDSVRG

SEQ ID NO: 186 - protein, 9 aa, synthetic construct
LGGWRHLDY

SEQ ID NO: 187 - protein, 11 aa, synthetic construct
RASQSIDNYLN

SEQ ID NO: 188 - protein, 7 aa, synthetic construct
AASSLQS

SEQ ID NO: 189 - protein, 9 aa, synthetic construct
QQSYSSPYT

SEQ ID NO: 190 - protein, 10 aa, synthetic construct
GLTFSNSWMS

SEQ ID NO: 191 - protein, 17 aa, synthetic construct
NINQDAGQKYSVDSVRG

SEQ ID NO: 192 - protein, 9 aa, synthetic construct
LGGWRHLDY

SEQ ID NO: 193 - protein, 107 aa, synthetic construct
DIQMTQSPSSLSASVGDRVTITCRASQSIDNYLNWYQQKSGKAPDLLIYAASSLQSGVPSRFSGSGSGTDFTLTISSLQPEDFATYYCQQSYSSPYTFGQGTKLEIK

SEQ ID NO: 194 - protein, 118 aa, synthetic construct
EVQLVESGGGLVQPGGSLRLSCAASGLTFSNSWMSWVRQTPGKGLEWVANINQDAGQKYSVDSVRGRFTISRDNAKNSLYLQMNSLRAEDTAVYYCARLGGWRHLDYWGQGTLVTVSS

SEQ ID NO: 195 - protein, 107 aa, synthetic construct; identical to SEQ ID NO: 193

SEQ ID NO: 196 - protein, 118 aa, synthetic construct; identical to SEQ ID NO: 194

SEQ ID NO: 197 - protein, 214 aa, synthetic construct
DIQMTQSPSSLSASVGDRVTITCRASQSIDNYLNWYQQKSGKAPDLLIYAASSLQSGVPSRFSGSGSGTDFTLTISSLQPEDFATYYCQQSYSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 198 - protein, 448 aa, synthetic construct
EVQLVESGGGLVQPGGSLRLSCAASGLTFSNSWMSWVRQTPGKGLEWVANINQDAGQKYSVDSVRGRFTISRDNAKNSLYLQMNSLRAEDTAVYYCARLGGWRHLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 199 - protein, 211 aa, synthetic construct
DIQMTQSPSSLSASVGDRVTITCRASQSIDNYLNWYQQKSGKAPDLLIYAASSLQSGVPSRFSGSGSGTDFTLTISSLQPEDFATYYCQQSYSSPYTFGQGTKLEIKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 200 - protein, 441 aa, synthetic construct
EVQLVESGGGLVQPGGSLRLSCAASGLTFSNSWMSWVRQTPGKGLEWVANINQDAGQKYSVDSVRGRFTISRDNAKNSLYLQMNSLRAEDTAVYYCARLGGWRHLDYWGQGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 201 - protein, 11 aa, synthetic construct
QASQSISSYLA

SEQ ID NO: 202 - protein, 7 aa, synthetic construct
RASTLTS

SEQ ID NO: 203 - protein, 13 aa, synthetic construct
QCTGGEICVKGRT

SEQ ID NO: 204 - protein, 11 aa, synthetic construct
GFSFSGVYDIC

SEQ ID NO: 205 - protein, 17 aa, synthetic construct
CIYIVTANPYYATWAKG

SEQ ID NO: 206 - protein, 11 aa, synthetic construct
GGDSTAWSWEL

SEQ ID NO: 207 - protein, 11 aa, synthetic construct
QASQSISSYLA

SEQ ID NO: 208 - protein, 7 aa, synthetic construct
RASTLTS

SEQ ID NO: 209 - protein, 13 aa, synthetic construct
QCTGGEICVKGRT

SEQ ID NO: 210 - protein, 11 aa, synthetic construct
GFSFSGVYDIC

SEQ ID NO: 211 - protein, 17 aa, synthetic construct
CIYIVTANPYYATWAKG

SEQ ID NO: 212 - protein, 11 aa, synthetic construct
GGDSTAWSWEL

SEQ ID NO: 213 - protein, 111 aa, synthetic construct
AFELTQTPASVEAAVGGTVTIKCQASQSISSYLAWYQQKPGQPPKLLIYRASTLTSGVPSRFRGSGSGTEFTLTISDLECADAATYYCQCTGGEICVKGRTFGGGTEVVVE

SEQ ID NO: 214 - protein, 120 aa, synthetic construct
QEQLEESGGGLVQPEGSLTLTCTASGFSFSGVYDICWVRQAPGKGPEWIACIYIVTANPYYATWAKGRFTMSKTSSTTVTLQLNSLTAADTATYFCVRGGDSTAWSWELWGPGTLVTVSS

SEQ ID NO: 215 - protein, 111 aa, synthetic construct; identical to SEQ ID NO: 213

SEQ ID NO: 216 - protein, 120 aa, synthetic construct; identical to SEQ ID NO: 214

SEQ ID NO: 217 - protein, 218 aa, synthetic construct
AFELTQTPASVEAAVGGTVTIKCQASQSISSYLAWYQQKPGQPPKLLIYRASTLTSGVPSRFRGSGSGTEFTLTISDLECADAATYYCQCTGGEICVKGRTFGGGTEVVVERTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 218 - protein, 450 aa, synthetic construct
QEQLEESGGGLVQPEGSLTLTCTASGFSFSGVYDICWVRQAPGKGPEWIACIYIVTANPYYATWAKGRFTMSKTSSTTVTLQLNSLTAADTATYFCVRGGDSTAWSWELWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 219 - protein, 215 aa, synthetic construct
AFELTQTPASVEAAVGGTVTIKCQASQSISSYLAWYQQKPGQPPKLLIYRASTLTSGVPSRFRGSGSGTEFTLTISDLECADAATYYCQCTGGEICVKGRTFGGGTEVVVEGDPVAPTVLIFPPAADQVATGTVTIVCVANKYFPDVTVTWEVDGTTQQSGIENSKTPQNSADCTYNLSSTLTLTSTQYNSHKEYTCKVTQGTTSVVQSFNRGDC

SEQ ID NO: 220 - protein, 443 aa, synthetic construct
QEQLEESGGGLVQPEGSLTLTCTASGFSFSGVYDICWVRQAPGKGPEWIACIYIVTANPYYATWAKGRFTMSKTSSTTVTLQLNSLTAADTATYFCVRGGDSTAWSWELWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPTVLDSDGSYFLYSKLSVPTSEWQRGDVFTCSVMHEALHNHYTQKSISRSPGK

SEQ ID NO: 221 - protein, 13 aa, synthetic construct
QSSQSVYSNNLLS

SEQ ID NO: 222 - protein, 7 aa, synthetic construct
RASTLAS

SEQ ID NO: 223 - protein, 10 aa, synthetic construct
GAVYSGYIYG

SEQ ID NO: 224 - protein, 10 aa, synthetic construct
GFSLITYTVI

SEQ ID NO: 225 - protein, 16 aa, synthetic construct
AIRSGESISYASWAKG

SEQ ID NO: 226 - protein, 7 aa, synthetic construct
SGNGFDS

SEQ ID NO: 227 - protein, 13 aa, synthetic construct
QSSQSVYSNNLLS

SEQ ID NO: 228 - protein, 7 aa, synthetic construct
RASTLAS

SEQ ID NO: 229 - protein, 10 aa, synthetic construct
GAVYSGYIYG

SEQ ID NO: 230 - protein, 10 aa, synthetic construct
GFSLITYTVI

SEQ ID NO: 231 - protein, 16 aa, synthetic construct
AIRSGESISYASWAKG

SEQ ID NO: 232 - protein, 7 aa, synthetic construct
SGNGFDS

SEQ ID NO: 233 - protein, 110 aa, synthetic construct
DPVMTQTPSSTSAAVGGTVTINCQSSQSVYSNNLLSWYQQKPGQPPKLLIYRASTLASGVPSRFKGSGSGTQFSLTISDLECDDVATYYCGAVYSGYIYGFGGGTEVVVK

SEQ ID NO: 234 - protein, 112 aa, synthetic construct
QSVEESGGRLVTPGTPLTLTCTVSGFSLITYTVIWVRQAPGKGLEWIGAIRSGESISYASWAKGRFTISETSTTVDLKITSPTTEDTATYFCTRSGNGFDSWGPGTLVTVSS

SEQ ID NO: 235 - protein, 110 aa, synthetic construct; identical to SEQ ID NO: 233

SEQ ID NO: 236 - protein, 112 aa, synthetic construct; identical to SEQ ID NO: 234

SEQ ID NO: 237 - protein, 217 aa, synthetic construct
DPVMTQTPSSTSAAVGGTVTINCQSSQSVYSNNLLSWYQQKPGQPPKLLIYRASTLASGVPSRFKGSGSGTQFSLTISDLECDDVATYYCGAVYSGYIYGFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 238 - protein, 442 aa, synthetic construct
QSVEESGGRLVTPGTPLTLTCTVSGFSLITYTVIWVRQAPGKGLEWIGAIRSGESISYASWAKGRFTISETSTTVDLKITSPTTEDTATYFCTRSGNGFDSWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 239 - protein, 214 aa, synthetic construct
DPVMTQTPSSTSAAVGGTVTINCQSSQSVYSNNLLSWYQQKPGQPPKLLIYRASTLASGVPSRFKGSGSGTQFSLTISDLECDDVATYYCGAVYSGYIYGFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 240 - protein, 435 aa, synthetic construct
QSVEESGGRLVTPGTPLTLTCTVSGFSLITYTVIWVRQAPGKGLEWIGAIRSGESISYASWAKGRFTISETSTTVDLKITSPTTEDTATYFCTRSGNGFDSWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 241 - protein, 11 aa, synthetic construct
QASESVGDRLA

SEQ ID NO: 242 - protein, 7 aa, synthetic construct
YIATLAS

SEQ ID NO: 243 - protein, 12 aa, synthetic construct
QYTNYDSVSGNV

SEQ ID NO: 244 - protein, 11 aa, synthetic construct
GIDFSGYVYMC

SEQ ID NO: 245 - protein, 18 aa, synthetic construct
CIDTDGSGRTWYASWAKG

SEQ ID NO: 246 - protein, 11 aa, synthetic construct
ETRSTGFSFNL

SEQ ID NO: 247 - protein, 11 aa, synthetic construct
QASESVGDRLA

SEQ ID NO: 248 - protein, 7 aa, synthetic construct
YIATLAS

SEQ ID NO: 249 - protein, 12 aa, synthetic construct
QYTNYDSVSGNV

SEQ ID NO: 250 - protein, 11 aa, synthetic construct
GIDFSGYVYMC

SEQ ID NO: 251 - protein, 18 aa, synthetic construct
CIDTDGSGRTWYASWAKG

SEQ ID NO: 252 - protein, 11 aa, synthetic construct
ETRSTGFSFNL

SEQ ID NO: 253 - protein, 110 aa, synthetic construct
DIVMTQTPASVSAAVGGTVTIKCQASESVGDRLAWYQQKPGQPPKLLIYYIATLASGVSSRFKGSGSGTQFTLTISDLECDDAATYYCQYTNYDSVSGNVFGGGTEVVVR

SEQ ID NO: 254 - protein, 118 aa, synthetic construct
QSLEESGGDLVKPGASLTLTCTASGIDFSGYVYMCWVRQAPGKGLEWITCIDTDGSGRTWYASWAKGRFTISKTSTTVTLQMASLTAADTATYFCARETRSTGFSFNWGPGTLVTVSS

SEQ ID NO: 255 - protein, 110 aa, synthetic construct; identical to SEQ ID NO: 253

SEQ ID NO: 256 - protein, 119 aa, synthetic construct
QSLEESGGDLVKPGASLTLTCTASGIDFSGYVYMCWVRQAPGKGLEWITCIDTDGSGRTWYASWAKGRFTISKTSTTVTLQMASLTAADTATYFCARETRSTGFSFNLWGPGTLVTVSS

SEQ ID NO: 257 - protein, 217 aa, synthetic construct
DIVMTQTPASVSAAVGGTVTIKCQASESVGDRLAWYQQKPGQPPKLLIYYIATLASGVSSRFKGSGSGTQFTLTISDLECDDAATYYCQYTNYDSVSGNVFGGGTEVVVRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 258 - protein, 449 aa, synthetic construct
QSLEESGGDLVKPGASLTLTCTASGIDFSGYVYMCWVRQAPGKGLEWITCIDTDGSGRTWYASWAKGRFTISKTSTTVTLQMASLTAADTATYFCARETRSTGFSFNLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 259 - protein, 214 aa, synthetic construct
DIVMTQTPASVSAAVGGTVTIKCQASESVGDRLAWYQQKPGQPPKLLIYYIATLASGVSSRFKGSGSGTQFTLTISDLECDDAATYYCQYTNYDSVSGNVFGGGTEVVVRGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 260 - protein, 442 aa, synthetic construct
QSLEESGGDLVKPGASLTLTCTASGIDFSGYVYMCWVRQAPGKGLEWITCIDTDGSGRTWYASWAKGRFTISKTSTTVTLQMASLTAADTATYFCARETRSTGFSFNLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 261 - protein, 13 aa, synthetic construct
QASQSVVDNNWLA

SEQ ID NO: 262 - protein, 7 aa, synthetic construct
RASNLAA

SEQ ID NO: 263 - protein, 10 aa, synthetic construct
GAHVSFGIFG

SEQ ID NO: 264 - protein, 10 aa, synthetic construct
GFSLSNHGVN

SEQ ID NO: 265 - protein, 16 aa, synthetic construct
IINGNGGTWYASWAKG

SEQ ID NO: 266 - protein, 10 aa, synthetic construct
GGAGAADSDI

SEQ ID NO: 267 - protein, 13 aa, synthetic construct
QASQSVVDNNWLA

SEQ ID NO: 268 - protein, 7 aa, synthetic construct
RASNLAA

SEQ ID NO: 269 - protein, 10 aa, synthetic construct
GAHVSFGIFG

SEQ ID NO: 270 - protein, 10 aa, synthetic construct
GFSLSNHGVN

SEQ ID NO: 271 - protein, 16 aa, synthetic construct
IINGNGGTWYASWAKG

SEQ ID NO: 272 - protein, 10 aa, synthetic construct
GGAGAADSDI

SEQ ID NO: 273 - protein, 110 aa, synthetic construct
AQVLTQTPSSTSAAVGGTVTINCQASQSVVDNNWLAWYQQKPGQPPKQLIYRASNLAAGVPSRFKGSGSGPQFTLTISDLECDDAATYFCGAHVSFGIFGFGGGTEVVVK

SEQ ID NO: 274 - protein, 115 aa, synthetic construct
QSVEESGGRLVTPGTPLTLTCTVSGFSLSNHGVNWVRQAPGKGLEWIGIINGNGGTWYASWAKGRFTISKTSTTVDLKMTSPTTEDTATYFCARGGAGAADSDIWGPGTLVTVSL

SEQ ID NO: 275 - protein, 110 aa, synthetic construct; identical to SEQ ID NO: 273

SEQ ID NO: 276 - protein, 115 aa, synthetic construct; identical to SEQ ID NO: 274

SEQ ID NO: 277 - protein, 217 aa, synthetic construct
AQVLTQTPSSTSAAVGGTVTINCQASQSVVDNNWLAWYQQKPGQPPKQLIYRASNLAAGVPSRFKGSGSGPQFTLTISDLECDDAATYFCGAHVSFGIFGFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 278 - protein, 445 aa, synthetic construct
QSVEESGGRLVTPGTPLTLTCTVSGFSLSNHGVNWVRQAPGKGLEWIGIINGNGGTWYASWAKGRFTISKTSTTVDLKMTSPTTEDTATYFCARGGAGAADSDIWGPGTLVTVSLASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 279 - protein, 214 aa, synthetic construct
AQVLTQTPSSTSAAVGGTVTINCQASQSVVDNNWLAWYQQKPGQPPKQLIYRASNLAAGVPSRFKGSGSGPQFTLTISDLECDDAATYFCGAHVSFGIFGFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 280 - protein, 438 aa, synthetic construct
QSVEESGGRLVTPGTPLTLTCTVSGFSLSNHGVNWVRQAPGKGLEWIGIINGNGGTWYASWAKGRFTISKTSTTVDLKMTSPTTEDTATYFCARGGAGAADSDIWGPGTLVTVSLGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 281 - protein, 11 aa, synthetic construct
QASESIGSYLS

SEQ ID NO: 282 - protein, 7 aa, synthetic construct
YASTLAS

SEQ ID NO: 283 - protein, 12 aa, synthetic construct
AGYKNWINDEYP

SEQ ID NO: 284 - protein, 10 aa, synthetic construct
GFSLSAYDMS

SEQ ID NO: 285 - protein, 16 aa, synthetic construct
IIDTGGSAYYMNWAKG

SEQ ID NO: 286 - protein, 13 aa, synthetic construct
VPWSSDSGSYLNL

SEQ ID NO: 287 - protein, 11 aa, synthetic construct
QASESIGSYLS

SEQ ID NO: 288 - protein, 7 aa, synthetic construct
YASTLAS

SEQ ID NO: 289 - protein, 12 aa, synthetic construct
AGYKNWINDEYP

SEQ ID NO: 290 - protein, 10 aa, synthetic construct
GFSLSAYDMS

SEQ ID NO: 291 - protein, 16 aa, synthetic construct
IIDTGGSAYYMNWAKG

SEQ ID NO: 292 - protein, 13 aa, synthetic construct
VPWSSDSGSYLNL

SEQ ID NO: 293 - protein, 110 aa, synthetic construct
ALVMTQTPSPVSAAVGSTVTISCQASESIGSYLSWYQQKPGQPPKLLIYYASTLASGVPSRFSGSGSGTQFTLTISGVECDDAATYYCAGYKNWINDEYPFGGGTEVVVK

SEQ ID NO: 294 - protein, 118 aa, synthetic construct
QSVEESGGRLVTPGTPLTLTCTASGFSLSAYDMSWVRQAPGKGLEWIGIIDTGGSAYYMNWAKGRFTISRTSTAVDLKMTSLTTEDTATYFCARVPWSSDSGSYLNLWGPGTLVTVSS

SEQ ID NO: 295 - protein, 110 aa, synthetic construct; identical to SEQ ID NO: 293

SEQ ID NO: 296 - protein, 118 aa, synthetic construct; identical to SEQ ID NO: 294

SEQ ID NO: 297 - protein, 217 aa, synthetic construct
ALVMTQTPSPVSAAVGSTVTISCQASESIGSYLSWYQQKPGQPPKLLIYYASTLASGVPSRFSGSGSGTQFTLTISGVECDDAATYYCAGYKNWINDEYPFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 298 - protein, 448 aa, synthetic construct
QSVEESGGRLVTPGTPLTLTCTASGFSLSAYDMSWVRQAPGKGLEWIGIIDTGGSAYYMNWAKGRFTISRTSTAVDLKMTSLTTEDTATYFCARVPWSSDSGSYLNLWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 299 - protein, 214 aa, synthetic construct
ALVMTQTPSPVSAAVGSTVTISCQASESIGSYLSWYQQKPGQPPKLLIYYASTLASGVPSRFSGSGSGTQFTLTISGVECDDAATYYCAGYKNWINDEYPFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 300 - protein, 441 aa, synthetic construct
QSVEESGGRLVTPGTPLTLTCTASGFSLSAYDMSWVRQAPGKGLEWIGIIDTGGSAYYMNWAKGRFTISRTSTAVDLKMTSLTTEDTATYFCARVPWSSDSGSYLNLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

(19) United States
(12) Patent Application Publication
(10) Pub. No.: US 2024/0337656 A1
(43) Pub. Date: Oct. 10, 2024

(54) STREPTOCOCCUS PNEUMONIAE SEROTYPE-SPECIFIC DETECTION ASSAY, REAGENTS AND KITS

(71) Applicant: Merck Sharp & Dohme LLC, Rahway, NJ (US)

(72) Inventors: Zhifeng Chen, Warrington, PA (US); Kara S. Cox, Harleysville, PA (US); Katrina M. Nolan, West Chester, PA (US); Gowrisankar Rajam, Doylestown, PA (US); Jeanette F. E. Roman, Quakertown, PA (US); Mary Lillian Shank-Retzlaff, Norristown, PA (US); Thorsten Verch, Blue Bell, PA (US); Kalpit A. Vora, Yardley, PA (US)

(73) Assignee: Merck Sharp & Dohme LLC, Rahway, NJ (US)

(21) Appl. No.: 18/625,385

(22) Filed: Apr. 3, 2024

Related U.S. Application Data
(60) Provisional application No. 63/457,254, filed on Apr. 5, 2023.

(51) Int. Cl. G01N 33/569; C07K 16/12; G01N 33/543

(52) U.S. Cl. CPC G01N 33/56944; C07K 16/1275; G01N 33/54326; C07K 2317/92; G01N 2333/3156; G01N 2469/10

(57) ABSTRACT

S. pneumoniae is a major cause of community-acquired pneumonia (CAP) in young children, older adults, and those with conditions or medications that compromise their immunity. Since the introduction of pneumococcal vaccines, the disease burden of vaccine serotypes (STs) on invasive pneumococcal disease has reduced; however, the effect on the burden of CAP is less well known, potentially due to a lack of testing for pneumococcal STs.

### CROSS-REFERENCE TO RELATED APPLICATIONS

This application claims the benefit of priority to U.S. Provisional Patent Application No. 63/457,254, filed Apr. 5, 2023.

### REFERENCE TO SEQUENCE LISTING SUBMITTED ELECTRONICALLY

The instant application contains a Sequence Listing which has been submitted electronically in XML format and is hereby incorporated by reference in its entirety. The XML file, created on Aug. 16, 2022, is named 25390USNP_SL.xml and is 285,463 bytes in size.

### FIELD OF THE INVENTION

The invention described herein relates to a high-throughput and multiplex Streptococcus pneumoniae (S. pneumoniae) serotype (ST)-specific urine antigen detection (SSUAD) assay, reagents and kit useful to support epidemiology studies, clinical trials and clinical therapy.

### BACKGROUND OF THE INVENTION

Despite vaccine availability, S. pneumoniae continues to be a major respiratory pathogen in children and older adults (Centers for Disease Control and Prevention. The Pink Book: epidemiology and prevention of vaccine-preventable diseases. 14th Edition. Chapter 17: pneumococcal disease. www.cdc.gov/vaccines/pubs/pinkbook/pneumo.html. Accessed Aug. 23, 2021 and European Centre for Disease Prevention and Control. Disease factsheet about pneumococcal disease. ecdc.europa.eu/en/pneumococcal-disease/facts. Accessed Aug. 16, 2021). Pneumococcus exhibits capsular polysaccharide diversity, drug resistance, and ability to cause a spectrum of diseases from otitis media to potentially life-threatening meningitis, contributing to their role as a major public health burden (Balsells E. et al., Serotype distribution of S. pneumoniae causing invasive disease in children in the post-PCV era: a systematic review and meta-analysis. PLOS One. 2017; 12:e0177113; Centers for Disease Control and Prevention. Surveillance and reporting. www.cdc.gov/pneumococcal/surveillance.html. Accessed Apr. 3, 2019; Løvlie A. et al., Changes in pneumococcal carriage prevalence and factors associated with carriage in Norwegian children, four years after introduction of PCV13. BMC Infect Dis. 2020; 20:29; and Savulescu C. et al., Effect of high-valency pneumococcal conjugate vaccines on invasive pneumococcal disease in children in SpIDnet countries: an observational multicentre study. Lancet Respir Med. 2017; 5:648-656). Structural differences in the capsule polysaccharide, a key virulence factor of S. pneumoniae, are used to categorize pneumococcal STs, with more than 90 distinct STs identified to date (World Health Organization. The weekly epidemiological record No 8, 2019, 94, 85-104. apps.who.int/iris/bitstream/handle/10665/310968/WER9408.pdf?ua=1. Accessed Apr. 3, 2019). The burden and ST distribution of non-bacteremic and non-invasive pneumococcal diseases and conditions, accounting for approximately 75% of pneumococcal pneumonia cases, are grossly underestimated, primarily due to the lack of in vitro assays (Said M. A. et al., Estimating the burden of pneumococcal pneumonia among adults: a systematic review and meta-analysis of diagnostic techniques. PLOS One. 2013; 8:e60273.).

A pneumococcal polysaccharide (PnPs) vaccine covering 23 STs (PPSV23), as well as pneumococcal conjugate vaccines (PCVs) covering 10 or 13 STs (PCV10 and PCV13), are currently available (World Health Organization. 23-valent pneumococcal polysaccharide vaccine. WHO position paper. Weekly Epidemiological Record. 2008; 83:373-384 and World Health Organization. Pneumococcal conjugate vaccines in infants and children under 5 years of age: WHO position paper—February 2019. Weekly Epidemiological Record. 2019; 94:85-104). PCV13 (Prevnar 13™, Pfizer, Inc. Philadelphia, PA, USA) is currently used in the childhood immunization schedule, while PPSV23 (Pneumovax 23™, Merck Sharp & Dohme Corp., Kenilworth, NJ, USA) is recommended for older adults and children >2 years of age with high-risk conditions in many European countries and the United States (van der Linden M. et al., Effects of infant pneumococcal conjugate vaccination on serotype distribution in invasive pneumococcal disease among children and adults in Germany. PLOS One. 2015; 10:00131494; Castiglia P. Recommendations for pneumococcal immunization outside routine childhood immunization programs in Western Europe. Adv Ther. 2014; 31:1011-1044; Matanock A. et al., Use of 13-valent pneumococcal conjugate vaccine and 23-valent pneumococcal polysaccharide vaccine among adults aged >/=65 years: updated recommendations of the Advisory Committee on Immunization Practices. MMWR Morb Mortal Wkly Rep. 2019; 68:1069-1075; and Kobayashi M. et al., Intervals between PCV13 and PPSV23 vaccines: recommendations of the Advisory Committee on Immunization Practices (ACIP). MMWR Morb Mortal Wkly Rep. 2015; 64:944-947). A 20-valent PCV (PCV20) (Prevnar 20™, Pfizer, Inc. Philadelphia, PA, USA) and a 15-valent PCV (PCV15) (Vaxneuvance™, Merck Sharp & Dohme Corp., Kenilworth, NJ, USA) have recently been approved in the United States by the Food and Drug Administration (FDA) for adults ≥18 years of age; both offer broader ST coverage than PCV13 (Prevnar 13™) (Food and Drug Administration. PREVNAR 20 (Pneumococcal 20-valent Conjugate Vaccine) Prescribing Information. www.fda.gov/media/149987/download. Accessed 26 Jul. 2021 and Food and Drug Administration. VAXNEUVANCE™ (Pneumococcal 15-valent Conjugate Vaccine) Prescribing Information. www.fda.gov/media/150819/download. Accessed 21 Jul. 2021).

Sensitive and specific assays are essential to determine the burden of PD, especially in conditions such as community-acquired pneumonia (CAP). Currently, pneumococcal CAP is mostly diagnosed by non-specific tests, such as chest X-rays and empirical clinical symptoms (Metlay J. P. et al., Diagnosis and treatment of adults with community-acquired pneumonia. An official clinical practice guideline of the American Thoracic Society and Infectious Diseases Society of America. American journal of respiratory and critical care medicine. 2019; 200:e45-e67 and Pletz M. W. et al., The burden of pneumococcal pneumonia-experience of the German competence network CAPNETZ. Pneumologie. 2012; 66:470-475). Occasionally, BinaxNOW™ (Abbott Diagnostics, Scarborough, Maine), a urine antigen test that detects pneumococcal common cell wall polysaccharide in a ST agonistic manner, is used for CAP diagnosis (Sinclair A. et al., Systematic review and meta-analysis of a urine-based pneumococcal antigen test for diagnosis of community-acquired pneumonia caused by S. pneumoniae. Journal of clinical microbiology. 2013; 51:2303-2310). Routine ST identification in patients with IPD is done via microbiological culture of clinical samples followed by a highly subjective latex agglutination and/or Quellung test, both of which have low sensitivity and require viable bacterial samples (Pride M. W. et al., Validation of an immunodiagnostic assay for detection of 13 S. pneumoniae serotype-specific polysaccharides in human urine. Clin Vaccine Immunol. 2012; 19:1131-1141; Said M. A. et al., Estimating the burden of pneumococcal pneumonia among adults: a systematic review and meta-analysis of diagnostic techniques. PLoS One. 2013; 8:e60273; Kuch A. et al., Usefulness of Pneumotest-latex for direct serotyping of S. pneumoniae isolates in clinical samples. Journal of clinical microbiology. 2014; 52:2647-2649; and Vissers M. et al., Increased carriage of non-vaccine serotypes with low invasive disease potential four years after switching to the 10-valent pneumococcal conjugate vaccine in The Netherlands. PLOS One. 2018; 13:00194823). Highly accurate ST identification can be achieved with polymerase chain reaction (PCR) and whole genome analysis, but these are not generally amenable to rapid or repeated testing to support large-scale surveillance studies or clinical trials (Public Health England. Guidelines for the public health management of clusters of severe pneumococcal disease in closed settings-Updated January 2020. assets.publishing.service.gov.uk/government/uploads/system/uploads/attachment_data/file/867175/Pneumococccal_cluster_guidelines.pdf. Accessed Apr. 4, 2021). More recently, ST-specific monoclonal antibodies have been used to detect and identify the presence of ST-specific PnPs in clinical samples (Wunderink R. G. et al., Pneumococcal Community-Acquired Pneumonia Detected by Serotype-Specific Urinary Antigen Detection Assays. Clin Infect Dis. 2018; 66:1504-1510 and Kalina W V, Souza V, Wu K, Giardina P, McKeen A, Jiang Q, Tan C, French R, Ren Y, Belanger K, McElhiney S, Unnithan M, Cheng H, Mininni T, Giordano-Schmidt D, Gessner B D, Jansen K U, Pride M W. Qualification and Clinical Validation of an Immunodiagnostic Assay for Detecting 11 Additional S. pneumoniae Serotype-specific Polysaccharides in Human Urine. Clin Infect Dis. 2020; 71:e430-e438.).

### SUMMARY OF THE INVENTION

Herein we describe the development, qualification, and clinical validation of a high-throughput, multiplex, serotype-specific urine antigen detection (SSUAD) assay for the quantification of PnPs STs of S. pneumoniae (1, 3, 4, 5, 6A, 6B, 7F, 9V, 14, 18C, 19A, 19F, 22F, 23F, and 33F) covered by Vaxneuvance™, i.e. the “Vaxneuvance serotypes”. We report performance characteristics that establish SSUAD as a suitable method to detect ST-specific PnPs in adult urine to support epidemiology studies or clinical trials.

In one embodiment the invention provides a method for detecting the presence or absence of a pneumococcal serotype (ST) capsular polysaccharide (PnPs) in a sample, the method comprising:
a) contacting the sample with: a first monoclonal antibody (mAb), or a functional variant thereof, that binds the pneumococcal serotype (ST) capsular polysaccharide (PnPs) in the sample to form a first mAb-antigen complex;
b) contacting the sample with: a second monoclonal antibody (mAb), or a functional variant thereof, that binds the pneumococcal serotype (ST) capsular polysaccharide (PnPs) to form a first mAb-antigen-second mAb complex;
c) contacting the sample with: a third antibody that binds the second monoclonal antibody (mAb), or a functional variant thereof, wherein the third antibody is detectably coupled to a reporter molecule;
d) detecting the presence or absence of the first mAb-antigen-second mAb complex by detecting the reporter molecule;
wherein the presence of the first mAb-antigen-second mAb complex confirms the presence of a pneumococcal serotype (ST) capsular polysaccharide (PnPs); and
wherein the absence of the first mAb-antigen-second mAb complex is indicative of the absence of a pneumococcal serotype (ST) capsular polysaccharide (PnPs).

In another embodiment the invention provides the method above wherein the first monoclonal antibody, or a functional variant thereof, specifically binds to a particular pneumococcal serotype capsular polysaccharide selected from ST-1, ST-3, ST-4, ST-5, ST-6A, ST-6B, ST-7F, ST-9V, ST-14, ST-18C, ST-19A, ST-19F, ST-22F, ST-23F and ST-33F.

In another embodiment the invention provides the method above wherein the first monoclonal antibody, or a functional variant thereof, comprises:
a) six complementarity determining regions (CDRs) selected from the group consisting of SEQ. ID. NOs.: 1-6, 21-26, 41-46, 61-66, 81-86, 101-106, 121-126, 141-146, 161-166, 181-186, 201-206, 221-226, 241-246, 261-266, and 281-286;
b) a variable heavy chain and variable light chain sequence selected from the group consisting of SEQ ID NOs: 13-14, 33-34, 53-54, 73-74, 93-94, 113-114, 133-134, 153-154, 173-174, 193-194, 213-214, 233-234, 253-254, 273-274, and 293-294; or
c) a full length light and heavy chain sequence selected from the group consisting of SEQ ID NOs: 17-18, 37-38, 57-58, 77-78, 97-98, 117-118, 137-138, 157-158, 177-178, 197-198, 217-218, 237-238, 257-258, 277-278, and 297-298.

In another embodiment the invention provides the method above wherein the second monoclonal antibody, or a functional variant thereof, specifically binds to a particular pneumococcal serotype capsular polysaccharide selected from ST-1, ST-3, ST-4, ST-5, ST-6A, ST-6B, ST-7F, ST-9V, ST-14, ST-18C, ST-19A, ST-19F, ST-22F, ST-23F and ST-33F.

In another embodiment the invention provides the method above wherein the second monoclonal antibody, or a functional variant thereof, comprises:
a) six complementarity determining regions (CDRs) selected from the group consisting of SEQ. ID. NOs.: 7-12, 27-32, 47-52, 67-72, 87-92, 107-112, 127-132, 147-152, 167-172, 187-192, 207-212, 227-232, 247-252, 267-272, and 287-292;
b) a variable heavy chain and variable light chain sequence selected from the group consisting of SEQ ID NOs: 15-16, 35-36, 55-56, 75-76, 95-96, 115-116, 135-136, 155-156, 175-176, 195-196, 215-216, 235-236, 255-256, 275-276, and 295-296; or
c) a full length light and heavy chain sequence selected from the group consisting of SEQ ID NOs: 19-20, 39-40, 59-60, 79-80, 99-100, 119-120, 139-140, 159-160, 179-180, 199-200, 219-220, 239-240, 259-260, 279-280, and 299-300.

In another embodiment the invention provides the method above wherein the first monoclonal antibody (mAb) binds a S. pneumoniae ST-1 PnPs to form a first mAb-antigen complex, wherein the first mAb comprises the following six CDRs:
i. a light chain CDR 1 comprising an amino acid sequence of SEQ ID NO: 1, or a functional variant thereof;
ii. a light chain CDR 2 comprising an amino acid sequence of SEQ ID NO: 2, or a functional variant thereof;
iii. a light chain CDR 3 comprising an amino acid sequence of SEQ ID NO: 3, or a functional variant thereof;
iv. a heavy chain CDR 4 comprising an amino acid sequence of SEQ ID NO: 4, or a functional variant thereof;
v. a heavy chain CDR 5 comprising an amino acid sequence of SEQ ID NO: 5, or a functional variant thereof;
vi. a heavy chain CDR 6 comprising an amino acid sequence of SEQ ID NO: 6, or a functional variant thereof;
wherein the first mAb binds a S. pneumoniae ST-1 PnPs; and
the second monoclonal antibody (mAb) binds a S. pneumoniae ST-1 PnPs to form a first mAb-antigen-second mAb complex, wherein the second mAb comprises the following six CDRs:
i. a light chain CDR 1 comprising an amino acid sequence of SEQ ID NO: 7, or a functional variant thereof;
ii. a light chain CDR 2 comprising an amino acid sequence of SEQ ID NO: 8, or a functional variant thereof;
iii. a light chain CDR 3 comprising an amino acid sequence of SEQ ID NO: 9, or a functional variant thereof;
iv. a heavy chain CDR 4 comprising an amino acid sequence of SEQ ID NO: 10, or a functional variant thereof;
v. a heavy chain CDR 5 comprising an amino acid sequence of SEQ ID NO: 11, or a functional variant thereof;
vi. a heavy chain CDR 6 comprising an amino acid sequence of SEQ ID NO: 12, or a functional variant thereof;
wherein the second mAb binds a S. pneumoniae ST-1 PnPs.

In another embodiment the invention provides the method above wherein the first monoclonal antibody (mAb) binds a S. pneumoniae ST-1 PnPs to form a first mAb-antigen complex, wherein the first mAb comprises:
i. a variable light chain comprising an amino acid sequence of SEQ ID NO: 13, or a functional variant thereof; and
ii. a variable heavy chain comprising an amino acid sequence of SEQ ID NO: 14, or a functional variant thereof; and
the second monoclonal antibody (mAb) binds a S. pneumoniae ST-1 PnPs to form a first mAb-antigen-second mAb complex, wherein the second mAb comprises:
i. a variable light chain comprising an amino acid sequence of SEQ ID NO: 15, or a functional variant thereof; and
ii. a variable heavy chain comprising an amino acid sequence of SEQ ID NO: 16, or a functional variant thereof.

In another embodiment the invention provides the method above wherein the first monoclonal antibody (mAb) binds a S. pneumoniae ST-1 PnPs to form a first mAb-antigen complex, wherein the first mAb comprises:
i. a full length light chain comprising an amino acid sequence of SEQ ID NO: 17, or a functional variant thereof; and
ii. a full length heavy chain comprising an amino acid sequence of SEQ ID NO: 18, or a functional variant thereof; and
the second monoclonal antibody (mAb) binds a S. pneumoniae ST-1 PnPs to form a first mAb-antigen-second mAb complex, wherein the second mAb comprises:
i. a full length light chain comprising an amino acid sequence of SEQ ID NO: 19, or a functional variant thereof; and
ii. a full length heavy chain comprising an amino acid sequence of SEQ ID NO: 20, or a functional variant thereof.

In another embodiment the invention provides the method above wherein the first monoclonal antibody (mAb) binds a S. pneumoniae ST-3 PnPs to form a first mAb-antigen complex, wherein the first mAb comprises the following six CDRs:
i. a light chain CDR 1 comprising an amino acid sequence of SEQ ID NO: 21, or a functional variant thereof;
ii. a light chain CDR 2 comprising an amino acid sequence of SEQ ID NO: 22, or a functional variant thereof;
iii. a light chain CDR 3 comprising an amino acid sequence of SEQ ID NO: 23, or a functional variant thereof;
iv. a heavy chain CDR 4 comprising an amino acid sequence of SEQ ID NO: 24, or a functional variant thereof;
v. a heavy chain CDR 5 comprising an amino acid sequence of SEQ ID NO: 25, or a functional variant thereof;
vi. a heavy chain CDR 6 comprising an amino acid sequence of SEQ ID NO: 26, or a functional variant thereof;
wherein the first mAb binds a S. pneumoniae ST-3 PnPs; and
the second monoclonal antibody (mAb) binds a S. pneumoniae ST-3 PnPs to form a first mAb-antigen-second mAb complex, wherein the second mAb comprises the following six CDRs:
i. a light chain CDR 1 comprising an amino acid sequence of SEQ ID NO: 27, or a functional variant thereof;
ii. a light chain CDR 2 comprising an amino acid sequence of SEQ ID NO: 28, or a functional variant thereof;
iii. a light chain CDR 3 comprising an amino acid sequence of SEQ ID NO: 29, or a functional variant thereof;
iv. a heavy chain CDR 4 comprising an amino acid sequence of SEQ ID NO: 30, or a functional variant thereof;
v. a heavy chain CDR 5 comprising an amino acid sequence of SEQ ID NO: 31, or a functional variant thereof;
vi. a heavy chain CDR 6 comprising an amino acid sequence of SEQ ID NO: 32, or a functional variant thereof;
wherein the second mAb binds a S. pneumoniae ST-3 PnPs.

In another embodiment the invention provides the method above wherein the first monoclonal antibody (mAb) binds a S. pneumoniae ST-3 PnPs to form a first mAb-antigen complex, wherein the first mAb comprises:
i. a variable light chain comprising an amino acid sequence of SEQ ID NO: 33, or a functional variant thereof; and
ii. a variable heavy chain comprising an amino acid sequence of SEQ ID NO: 34, or a functional variant thereof; and
the second monoclonal antibody (mAb) binds a S. pneumoniae ST-3 PnPs to form a first mAb-antigen-second mAb complex, wherein the second mAb comprises:
i. a variable light chain comprising an amino acid sequence of SEQ ID NO: 35, or a functional variant thereof; and
ii. a variable heavy chain comprising an amino acid sequence of SEQ ID NO: 36, or a functional variant thereof.

In another embodiment the invention provides the method above wherein the first monoclonal antibody (mAb) binds a S. pneumoniae ST-3 PnPs to form a first mAb-antigen complex, wherein the first mAb comprises:
i. a full length light chain comprising an amino acid sequence of SEQ ID NO: 37, or a functional variant thereof; and
ii. a full length heavy chain comprising an amino acid sequence of SEQ ID NO: 38, or a functional variant thereof; and
the second monoclonal antibody (mAb) binds a S. pneumoniae ST-3 PnPs to form a first mAb-antigen-second mAb complex, wherein the second mAb comprises:
i. a full length light chain comprising an amino acid sequence of SEQ ID NO: 39, or a functional variant thereof; and
ii. a full length heavy chain comprising an amino acid sequence of SEQ ID NO: 40, or a functional variant thereof.

In another embodiment the invention provides the method above wherein the first monoclonal antibody (mAb) binds a S. pneumoniae ST-4 PnPs to form a first mAb-antigen complex, wherein the first mAb comprises the following six CDRs:
i. a light chain CDR 1 comprising an amino acid sequence of SEQ ID NO: 41, or a functional variant thereof;
ii. a light chain CDR 2 comprising an amino acid sequence of SEQ ID NO: 42, or a functional variant thereof;
iii. a light chain CDR 3 comprising an amino acid sequence of SEQ ID NO: 43, or a functional variant thereof;
iv. a heavy chain CDR 4 comprising an amino acid sequence of SEQ ID NO: 44, or a functional variant thereof;
v. a heavy chain CDR 5 comprising an amino acid sequence of SEQ ID NO: 45, or a functional variant thereof;
vi. a heavy chain CDR 6 comprising an amino acid sequence of SEQ ID NO: 46, or a functional variant thereof;
wherein the first mAb binds a S. pneumoniae ST-4 PnPs; and
the second monoclonal antibody (mAb) binds a S. pneumoniae ST-4 PnPs to form a first mAb-antigen-second mAb complex, wherein the second mAb comprises the following six CDRs:
i. a light chain CDR 1 comprising an amino acid sequence of SEQ ID NO: 47, or a functional variant thereof;
ii. a light chain CDR 2 comprising an amino acid sequence of SEQ ID NO: 48, or a functional variant thereof;
iii. a light chain CDR 3 comprising an amino acid sequence of SEQ ID NO: 49, or a functional variant thereof;
iv. a heavy chain CDR 4 comprising an amino acid sequence of SEQ ID NO: 50, or a functional variant thereof;
v. a heavy chain CDR 5 comprising an amino acid sequence of SEQ ID NO: 51, or a functional variant thereof;
vi. a heavy chain CDR 6 comprising an amino acid sequence of SEQ ID NO: 52, or a functional variant thereof;
wherein the second mAb binds a S. pneumoniae ST-4 PnPs.

In another embodiment the invention provides the method above wherein the first monoclonal antibody (mAb) binds a S. pneumoniae ST-4 PnPs to form a first mAb-antigen complex, wherein the first mAb comprises:
i. a variable light chain comprising an amino acid sequence of SEQ ID NO: 53, or a functional variant thereof; and
ii. a variable heavy chain comprising an amino acid sequence of SEQ ID NO: 54, or a functional variant thereof; and
the second monoclonal antibody (mAb) binds a S. pneumoniae ST-4 PnPs to form a first mAb-antigen-second mAb complex, wherein the second mAb comprises:
i. a variable light chain comprising an amino acid sequence of SEQ ID NO: 55, or a functional variant thereof; and
ii. a variable heavy chain comprising an amino acid sequence of SEQ ID NO: 56, or a functional variant thereof.

In another embodiment the invention provides the method above wherein the first monoclonal antibody (mAb) binds a S. pneumoniae ST-4 PnPs to form a first mAb-antigen complex, wherein the first mAb comprises:
i. a full length light chain comprising an amino acid sequence of SEQ ID NO: 57, or a functional variant thereof; and
ii. a full length heavy chain comprising an amino acid sequence of SEQ ID NO: 58, or a functional variant thereof; and
the second monoclonal antibody (mAb) binds a S. pneumoniae ST-4 PnPs to form a first mAb-antigen-second mAb complex, wherein the second mAb comprises:
i. a full length light chain comprising an amino acid sequence of SEQ ID NO: 59, or a functional variant thereof; and
ii. a full length heavy chain comprising an amino acid sequence of SEQ ID NO: 60, or a functional variant thereof.

In another embodiment the invention provides the method above wherein the first monoclonal antibody (mAb) binds a S. pneumoniae ST-5 PnPs to form a first mAb-antigen complex, wherein the first mAb comprises the following six CDRs:
i. a light chain CDR 1 comprising an amino acid sequence of SEQ ID NO: 61, or a functional variant thereof;
ii. a light chain CDR 2 comprising an amino acid sequence of SEQ ID NO: 62, or a functional variant thereof;
iii. a light chain CDR 3 comprising an amino acid sequence of SEQ ID NO: 63, or a functional variant thereof;
iv. a heavy chain CDR 4 comprising an amino acid sequence of SEQ ID NO: 64, or a functional variant thereof;
v. a heavy chain CDR 5 comprising an amino acid sequence of SEQ ID NO: 65, or a functional variant thereof;
vi. a heavy chain CDR 6 comprising an amino acid sequence of SEQ ID NO: 66, or a functional variant thereof;
wherein the first mAb binds a S. pneumoniae ST-5 PnPs; and
the second monoclonal antibody (mAb) binds a S. pneumoniae ST-5 PnPs to form a first mAb-antigen-second mAb complex, wherein the second mAb comprises the following six CDRs:
i. a light chain CDR 1 comprising an amino acid sequence of SEQ ID NO: 67, or a functional variant thereof;
ii. a light chain CDR 2 comprising an amino acid sequence of SEQ ID NO: 68, or a functional variant thereof;
iii. a light chain CDR 3 comprising an amino acid sequence of SEQ ID NO: 69, or a functional variant thereof;
iv. a heavy chain CDR 4 comprising an amino acid sequence of SEQ ID NO: 70, or a functional variant thereof;
v. a heavy chain CDR 5 comprising an amino acid sequence of SEQ ID NO: 71, or a functional variant thereof;
vi. a heavy chain CDR 6 comprising an amino acid sequence of SEQ ID NO: 72, or a functional variant thereof;
wherein the second mAb binds a S. pneumoniae ST-5 PnPs.

In another embodiment the invention provides the method above wherein the first monoclonal antibody (mAb) binds a S. pneumoniae ST-5 PnPs to form a first mAb-antigen complex, wherein the first mAb comprises:
i. a variable light chain comprising an amino acid sequence of SEQ ID NO: 73, or a functional variant thereof; and
ii. a variable heavy chain comprising an amino acid sequence of SEQ ID NO: 74, or a functional variant thereof; and
the second monoclonal antibody (mAb) binds a S. pneumoniae ST-5 PnPs to form a first mAb-antigen-second mAb complex, wherein the second mAb comprises:
i. a variable light chain comprising an amino acid sequence of SEQ ID NO: 75, or a functional variant thereof; and
ii. a variable heavy chain comprising an amino acid sequence of SEQ ID NO: 76, or a functional variant thereof.

In another embodiment the invention provides the method above wherein the first monoclonal antibody (mAb) binds a S. pneumoniae ST-5 PnPs to form a first mAb-antigen complex, wherein the first mAb comprises:
i. a full length light chain comprising an amino acid sequence of SEQ ID NO: 77, or a functional variant thereof; and
ii. a full length heavy chain comprising an amino acid sequence of SEQ ID NO: 78, or a functional variant thereof; and
the second monoclonal antibody (mAb) binds a S. pneumoniae ST-5 PnPs to form a first mAb-antigen-second mAb complex, wherein the second mAb comprises:
i. a full length light chain comprising an amino acid sequence of SEQ ID NO: 79, or a functional variant thereof; and
ii. a full length heavy chain comprising an amino acid sequence of SEQ ID NO: 80, or a functional variant thereof.

In another embodiment the invention provides the method above wherein the first monoclonal antibody (mAb) binds a S. pneumoniae ST-6A PnPs to form a first mAb-antigen complex, wherein the first mAb comprises the following six CDRs:
i. a light chain CDR 1 comprising an amino acid sequence of SEQ ID NO: 81, or a functional variant thereof;
ii. a light chain CDR 2 comprising an amino acid sequence of SEQ ID NO: 82, or a functional variant thereof;
iii. a light chain CDR 3 comprising an amino acid sequence of SEQ ID NO: 83, or a functional variant thereof;
iv. a heavy chain CDR 4 comprising an amino acid sequence of SEQ ID NO: 84, or a functional variant thereof;
v. a heavy chain CDR 5 comprising an amino acid sequence of SEQ ID NO: 85, or a functional variant thereof;
vi. a heavy chain CDR 6 comprising an amino acid sequence of SEQ ID NO: 86, or a functional variant thereof;
wherein the first mAb binds a S. pneumoniae ST-6A PnPs; and
the second monoclonal antibody (mAb) binds a S. pneumoniae ST-6A PnPs to form a first mAb-antigen-second mAb complex, wherein the second mAb comprises the following six CDRs:
i. a light chain CDR 1 comprising an amino acid sequence of SEQ ID NO: 87, or a functional variant thereof;
ii. a light chain CDR 2 comprising an amino acid sequence of SEQ ID NO: 88, or a functional variant thereof;
iii. a light chain CDR 3 comprising an amino acid sequence of SEQ ID NO: 89, or a functional variant thereof;
iv. a heavy chain CDR 4 comprising an amino acid sequence of SEQ ID NO: 90, or a functional variant thereof;
v. a heavy chain CDR 5 comprising an amino acid sequence of SEQ ID NO: 91, or a functional variant thereof;
vi. a heavy chain CDR 6 comprising an amino acid sequence of SEQ ID NO: 92, or a functional variant thereof;
wherein the second mAb binds a S. pneumoniae ST-6A PnPs.

In another embodiment the invention provides the method above wherein the first monoclonal antibody (mAb) binds a S. pneumoniae ST-6A PnPs to form a first mAb-antigen complex, wherein the first mAb comprises:
i. a variable light chain comprising an amino acid sequence of SEQ ID NO: 93, or a functional variant thereof; and
ii. a variable heavy chain comprising an amino acid sequence of SEQ ID NO: 94, or a functional variant thereof; and
the second monoclonal antibody (mAb) binds a S. pneumoniae ST-6A PnPs to form a first mAb-antigen-second mAb complex, wherein the second mAb comprises:
i. a variable light chain comprising an amino acid sequence of SEQ ID NO: 95, or a functional variant thereof; and
ii. a variable heavy chain comprising an amino acid sequence of SEQ ID NO: 96, or a functional variant thereof.

In another embodiment the invention provides the method above wherein the first monoclonal antibody (mAb) binds a S. pneumoniae ST-6A PnPs to form a first mAb-antigen complex, wherein the first mAb comprises:
i. a full length light chain comprising an amino acid sequence of SEQ ID NO: 97, or a functional variant thereof; and
ii. a full length heavy chain comprising an amino acid sequence of SEQ ID NO: 98, or a functional variant thereof; and
the second monoclonal antibody (mAb) binds a S. pneumoniae ST-6A PnPs to form a first mAb-antigen-second mAb complex, wherein the second mAb comprises:
i. a full length light chain comprising an amino acid sequence of SEQ ID NO: 99, or a functional variant thereof; and
ii. a full length heavy chain comprising an amino acid sequence of SEQ ID NO: 100, or a functional variant thereof.

In another embodiment the invention provides the method above wherein the first monoclonal antibody (mAb) binds a S. pneumoniae ST-6B PnPs to form a first mAb-antigen complex, wherein the first mAb comprises the following six CDRs:
i. a light chain CDR 1 comprising an amino acid sequence of SEQ ID NO: 101, or a functional variant thereof;
ii. a light chain CDR 2 comprising an amino acid sequence of SEQ ID NO: 102, or a functional variant thereof;
iii. a light chain CDR 3 comprising an amino acid sequence of SEQ ID NO: 103, or a functional variant thereof;
iv. a heavy chain CDR 4 comprising an amino acid sequence of SEQ ID NO: 104, or a functional variant thereof;
v. a heavy chain CDR 5 comprising an amino acid sequence of SEQ ID NO: 105, or a functional variant thereof;
vi. a heavy chain CDR 6 comprising an amino acid sequence of SEQ ID NO: 106, or a functional variant thereof;
wherein the first mAb binds a S. pneumoniae ST-6B PnPs; and
the second monoclonal antibody (mAb) binds a S. pneumoniae ST-6B PnPs to form a first mAb-antigen-second mAb complex, wherein the second mAb comprises the following six CDRs:
i. a light chain CDR 1 comprising an amino acid sequence of SEQ ID NO: 107, or a functional variant thereof;
ii. a light chain CDR 2 comprising an amino acid sequence of SEQ ID NO: 108, or a functional variant thereof;
iii. a light chain CDR 3 comprising an amino acid sequence of SEQ ID NO: 109, or a functional variant thereof;
iv. a heavy chain CDR 4 comprising an amino acid sequence of SEQ ID NO: 110, or a functional variant thereof;
v. a heavy chain CDR 5 comprising an amino acid sequence of SEQ ID NO: 111, or a functional variant thereof;
vi. a heavy chain CDR 6 comprising an amino acid sequence of SEQ ID NO: 112, or a functional variant thereof;
wherein the second mAb binds a S. pneumoniae ST-6B PnPs.

In another embodiment the invention provides the method above wherein the first monoclonal antibody (mAb) binds a S. pneumoniae ST-6B PnPs to form a first mAb-antigen complex, wherein the first mAb comprises:
i. a variable light chain comprising an amino acid sequence of SEQ ID NO: 113, or a functional variant thereof; and
ii. a variable heavy chain comprising an amino acid sequence of SEQ ID NO: 114, or a functional variant thereof; and
the second monoclonal antibody (mAb) binds a S. pneumoniae ST-6B PnPs to form a first mAb-antigen-second mAb complex, wherein the second mAb comprises:
i. a variable light chain comprising an amino acid sequence of SEQ ID NO: 115, or a functional variant thereof; and
ii. a variable heavy chain comprising an amino acid sequence of SEQ ID NO: 116, or a functional variant thereof.

In another embodiment the invention provides the method above wherein the first monoclonal antibody (mAb) binds a S. pneumoniae ST-6B PnPs to form a first mAb-antigen complex, wherein the first mAb comprises:
i. a full length light chain comprising an amino acid sequence of SEQ ID NO: 117, or a functional variant thereof; and
ii. a full length heavy chain comprising an amino acid sequence of SEQ ID NO: 118, or a functional variant thereof; and
the second monoclonal antibody (mAb) binds a S. pneumoniae ST-6B PnPs to form a first mAb-antigen-second mAb complex, wherein the second mAb comprises:
i. a full length light chain comprising an amino acid sequence of SEQ ID NO: 119, or a functional variant thereof; and
ii. a full length heavy chain comprising an amino acid sequence of SEQ ID NO: 120, or a functional variant thereof.

In another embodiment the invention provides the method above wherein the first monoclonal antibody (mAb) binds a S. pneumoniae ST-7F PnPs to form a first mAb-antigen complex, wherein the first mAb comprises the following six CDRs:
i. a light chain CDR 1 comprising an amino acid sequence of SEQ ID NO: 121, or a functional variant thereof;
ii. a light chain CDR 2 comprising an amino acid sequence of SEQ ID NO: 122, or a functional variant thereof;
iii. a light chain CDR 3 comprising an amino acid sequence of SEQ ID NO: 123, or a functional variant thereof;
iv. a heavy chain CDR 4 comprising an amino acid sequence of SEQ ID NO: 124, or a functional variant thereof;
v. a heavy chain CDR 5 comprising an amino acid sequence of SEQ ID NO: 125, or a functional variant thereof;
vi. a heavy chain CDR 6 comprising an amino acid sequence of SEQ ID NO: 126, or a functional variant thereof;
wherein the first mAb binds a S. pneumoniae ST-7F PnPs; and
the second monoclonal antibody (mAb) binds a S. pneumoniae ST-7F PnPs to form a first mAb-antigen-second mAb complex, wherein the second mAb comprises the following six CDRs:
i. a light chain CDR 1 comprising an amino acid sequence of SEQ ID NO: 127, or a functional variant thereof;
ii. a light chain CDR 2 comprising an amino acid sequence of SEQ ID NO: 128, or a functional variant thereof;
iii. a light chain CDR 3 comprising an amino acid sequence of SEQ ID NO: 129, or a functional variant thereof;
iv. a heavy chain CDR 4 comprising an amino acid sequence of SEQ ID NO: 130, or a functional variant thereof;
v. a heavy chain CDR 5 comprising an amino acid sequence of SEQ ID NO: 131, or a functional variant thereof;
vi. a heavy chain CDR 6 comprising an amino acid sequence of SEQ ID NO: 132, or a functional variant thereof;
wherein the second mAb binds a S. pneumoniae ST-7F PnPs.

In another embodiment the invention provides the method above wherein the first monoclonal antibody (mAb) binds a S. pneumoniae ST-7F PnPs to form a first mAb-antigen complex, wherein the first mAb comprises:
i. a variable light chain comprising an amino acid sequence of SEQ ID NO: 133, or a functional variant thereof; and
ii. a variable heavy chain comprising an amino acid sequence of SEQ ID NO: 134, or a functional variant thereof; and
the second monoclonal antibody (mAb) binds a S. pneumoniae ST-7F PnPs to form a first mAb-antigen-second mAb complex, wherein the second mAb comprises:
i. a variable light chain comprising an amino acid sequence of SEQ ID NO: 135, or a functional variant thereof; and
ii. a variable heavy chain comprising an amino acid sequence of SEQ ID NO: 136, or a functional variant thereof;

In another embodiment the invention provides the method above wherein the first monoclonal antibody (mAb) binds a S. pneumoniae ST-7F PnPs to form a first mAb-antigen complex, wherein the first mAb comprises:
i. a full length light chain comprising an amino acid sequence of SEQ ID NO: 137, or a functional variant thereof; and
ii. a full length heavy chain comprising an amino acid sequence of SEQ ID NO: 138, or a functional variant thereof; and
the second monoclonal antibody (mAb) binds a S. pneumoniae ST-7F PnPs to form a first mAb-antigen-second mAb complex, wherein the second mAb comprises:
i. a full length light chain comprising an amino acid sequence of SEQ ID NO: 139, or a functional variant thereof; and
ii. a full length heavy chain comprising an amino acid sequence of SEQ ID NO: 140, or a functional variant thereof.

In another embodiment the invention provides the method above wherein the first monoclonal antibody (mAb) binds a S. pneumoniae ST-9V PnPs to form a first mAb-antigen complex, wherein the first mAb comprises the following six CDRs:
i. a light chain CDR 1 comprising an amino acid sequence of SEQ ID NO: 141, or a functional variant thereof;
ii. a light chain CDR 2 comprising an amino acid sequence of SEQ ID NO: 142, or a functional variant thereof;
iii. a light chain CDR 3 comprising an amino acid sequence of SEQ ID NO: 143, or a functional variant thereof;
iv. a heavy chain CDR 4 comprising an amino acid sequence of SEQ ID NO: 144, or a functional variant thereof;
v. a heavy chain CDR 5 comprising an amino acid sequence of SEQ ID NO: 145, or a functional variant thereof;
vi. a heavy chain CDR 6 comprising an amino acid sequence of SEQ ID NO: 146, or a functional variant thereof;
wherein the first mAb binds a S. pneumoniae ST-9V PnPs; and
the second monoclonal antibody (mAb) binds a S. pneumoniae ST-9V PnPs to form a first mAb-antigen-second mAb complex, wherein the second mAb comprises the following six CDRs:
i. a light chain CDR 1 comprising an amino acid sequence of SEQ ID NO: 147, or a functional variant thereof;
ii. a light chain CDR 2 comprising an amino acid sequence of SEQ ID NO: 148, or a functional variant thereof;
iii. a light chain CDR 3 comprising an amino acid sequence of SEQ ID NO: 149, or a functional variant thereof;
iv. a heavy chain CDR 4 comprising an amino acid sequence of SEQ ID NO: 150, or a functional variant thereof;
v. a heavy chain CDR 5 comprising an amino acid sequence of SEQ ID NO: 151, or a functional variant thereof;
vi. a heavy chain CDR 6 comprising an amino acid sequence of SEQ ID NO: 152, or a functional variant thereof;
wherein the second mAb binds a S. pneumoniae ST-9V PnPs.

In another embodiment the invention provides the method above wherein the first monoclonal antibody (mAb) binds a S. pneumoniae ST-9V PnPs to form a first mAb-antigen complex, wherein the first mAb comprises:
i. a variable light chain comprising an amino acid sequence of SEQ ID NO: 153, or a functional variant thereof; and
ii. a variable heavy chain comprising an amino acid sequence of SEQ ID NO: 154, or a functional variant thereof; and
the second monoclonal antibody (mAb) binds a S. pneumoniae ST-9V PnPs to form a first mAb-antigen-second mAb complex, wherein the second mAb comprises:
i. a variable light chain comprising an amino acid sequence of SEQ ID NO: 155, or a functional variant thereof; and
ii. a variable heavy chain comprising an amino acid sequence of SEQ ID NO: 156, or a functional variant thereof.

In another embodiment the invention provides the method above wherein the first monoclonal antibody (mAb) binds a S. pneumoniae ST-9V PnPs to form a first mAb-antigen complex, wherein the first mAb comprises:
i. a full length light chain comprising an amino acid sequence of SEQ ID NO: 157, or a functional variant thereof; and
ii. a full length heavy chain comprising an amino acid sequence of SEQ ID NO: 158, or a functional variant thereof; and
the second monoclonal antibody (mAb) binds a S. pneumoniae ST-9V PnPs to form a first mAb-antigen-second mAb complex, wherein the second mAb comprises:
i. a full length light chain comprising an amino acid sequence of SEQ ID NO: 159, or a functional variant thereof; and
ii. a full length heavy chain comprising an amino acid sequence of SEQ ID NO: 160, or a functional variant thereof.

In another embodiment the invention provides the method above wherein the first monoclonal antibody (mAb) binds a S. pneumoniae ST-14 PnPs to form a first mAb-antigen complex, wherein the first mAb comprises the following six CDRs:
i. a light chain CDR 1 comprising an amino acid sequence of SEQ ID NO: 161, or a functional variant thereof;
ii. a light chain CDR 2 comprising an amino acid sequence of SEQ ID NO: 162, or a functional variant thereof;
iii. a light chain CDR 3 comprising an amino acid sequence of SEQ ID NO: 163, or a functional variant thereof;
iv. a heavy chain CDR 4 comprising an amino acid sequence of SEQ ID NO: 164, or a functional variant thereof;
v. a heavy chain CDR 5 comprising an amino acid sequence of SEQ ID NO: 165, or a functional variant thereof;
vi. a heavy chain CDR 6 comprising an amino acid sequence of SEQ ID NO: 166, or a functional variant thereof;
wherein the first mAb binds a S. pneumoniae ST-14 PnPs; and
the second monoclonal antibody (mAb) binds a S. pneumoniae ST-14 PnPs to form a first mAb-antigen-second mAb complex, wherein the second mAb comprises the following six CDRs:
i. a light chain CDR 1 comprising an amino acid sequence of SEQ ID NO: 167, or a functional variant thereof;
ii. a light chain CDR 2 comprising an amino acid sequence of SEQ ID NO: 168, or a functional variant thereof;
iii. a light chain CDR 3 comprising an amino acid sequence of SEQ ID NO: 169, or a functional variant thereof;
iv. a heavy chain CDR 4 comprising an amino acid sequence of SEQ ID NO: 170, or a functional variant thereof;
v. a heavy chain CDR 5 comprising an amino acid sequence of SEQ ID NO: 171, or a functional variant thereof;
vi. a heavy chain CDR 6 comprising an amino acid sequence of SEQ ID NO: 172, or a functional variant thereof;
wherein the second mAb binds a S. pneumoniae ST-14 PnPs.

In another embodiment the invention provides the method above wherein the first monoclonal antibody (mAb) binds a S. pneumoniae ST-14 PnPs to form a first mAb-antigen complex, wherein the first mAb comprises:
i. a variable light chain comprising an amino acid sequence of SEQ ID NO: 173, or a functional variant thereof; and
ii. a variable heavy chain comprising an amino acid sequence of SEQ ID NO: 174, or a functional variant thereof; and
the second monoclonal antibody (mAb) binds a S. pneumoniae ST-14 PnPs to form a first mAb-antigen-second mAb complex, wherein the second mAb comprises:
i. a variable light chain comprising an amino acid sequence of SEQ ID NO: 175, or a functional variant thereof; and
ii. a variable heavy chain comprising an amino acid sequence of SEQ ID NO: 176, or a functional variant thereof.

In another embodiment the invention provides the method above wherein the first monoclonal antibody (mAb) binds a S. pneumoniae ST-14 PnPs to form a first mAb-antigen complex, wherein the first mAb comprises:
i. a full length light chain comprising an amino acid sequence of SEQ ID NO: 177, or a functional variant thereof; and
ii. a full length heavy chain comprising an amino acid sequence of SEQ ID NO: 178, or a functional variant thereof; and
the second monoclonal antibody (mAb) binds a S. pneumoniae ST-14 PnPs to form a first mAb-antigen-second mAb complex, wherein the second mAb comprises:
i. a full length light chain comprising an amino acid sequence of SEQ ID NO: 179, or a functional variant thereof; and
ii. a full length heavy chain comprising an amino acid sequence of SEQ ID NO: 180, or a functional variant thereof.

In another embodiment the invention provides the method above wherein the first monoclonal antibody (mAb) binds a S. pneumoniae ST-18C PnPs to form a first mAb-antigen complex, wherein the first mAb comprises the following six CDRs:
i. a light chain CDR 1 comprising an amino acid sequence of SEQ ID NO: 181, or a functional variant thereof;
ii. a light chain CDR 2 comprising an amino acid sequence of SEQ ID NO: 182, or a functional variant thereof;
iii. a light chain CDR 3 comprising an amino acid sequence of SEQ ID NO: 183, or a functional variant thereof;
iv. a heavy chain CDR 4 comprising an amino acid sequence of SEQ ID NO: 184, or a functional variant thereof;
v. a heavy chain CDR 5 comprising an amino acid sequence of SEQ ID NO: 185, or a functional variant thereof;
vi. a heavy chain CDR 6 comprising an amino acid sequence of SEQ ID NO: 186, or a functional variant thereof;
wherein the first mAb binds a S. pneumoniae ST-18C PnPs; and
the second monoclonal antibody (mAb) binds a S. pneumoniae ST-18C PnPs to form a first mAb-antigen-second mAb complex, wherein the second mAb comprises the following six CDRs:
i. a light chain CDR 1 comprising an amino acid sequence of SEQ ID NO: 187, or a functional variant thereof;
ii. a light chain CDR 2 comprising an amino acid sequence of SEQ ID NO: 188, or a functional variant thereof;
iii. a light chain CDR 3 comprising an amino acid sequence of SEQ ID NO: 189, or a functional variant thereof;
iv. a heavy chain CDR 4 comprising an amino acid sequence of SEQ ID NO: 190, or a functional variant thereof;
v. a heavy chain CDR 5 comprising an amino acid sequence of SEQ ID NO: 191, or a functional variant thereof;
vi. a heavy chain CDR 6 comprising an amino acid sequence of SEQ ID NO: 192, or a functional variant thereof;
wherein the second mAb binds a S. pneumoniae ST-18C PnPs.

In another embodiment the invention provides the method above wherein the first monoclonal antibody (mAb) binds a S. pneumoniae ST-18C PnPs to form a first mAb-antigen complex, wherein the first mAb comprises:
i. a variable light chain comprising an amino acid sequence of SEQ ID NO: 193, or a functional variant thereof; and
ii. a variable heavy chain comprising an amino acid sequence of SEQ ID NO: 194, or a functional variant thereof; and
the second monoclonal antibody (mAb) binds a S. pneumoniae ST-18C PnPs to form a first mAb-antigen-second mAb complex, wherein the second mAb comprises:
i. a variable light chain comprising an amino acid sequence of SEQ ID NO: 195, or a functional variant thereof; and
ii. a variable heavy chain comprising an amino acid sequence of SEQ ID NO: 196, or a functional variant thereof.

In another embodiment the invention provides the method above wherein the first monoclonal antibody (mAb) binds a S. pneumoniae ST-18C PnPs to form a first mAb-antigen complex, wherein the first mAb comprises:
i. a full length light chain comprising an amino acid sequence of SEQ ID NO: 197, or a functional variant thereof; and
ii. a full length heavy chain comprising an amino acid sequence of SEQ ID NO: 198, or a functional variant thereof; and
the second monoclonal antibody (mAb) binds a S. pneumoniae ST-18C PnPs to form a first mAb-antigen-second mAb complex, wherein the second mAb comprises:
i. a full length light chain comprising an amino acid sequence of SEQ ID NO: 199, or a functional variant thereof; and
ii. a full length heavy chain comprising an amino acid sequence of SEQ ID NO: 200, or a functional variant thereof.

In another embodiment the invention provides the method above wherein the first monoclonal antibody (mAb) binds a S. pneumoniae ST-19A PnPs to form a first mAb-antigen complex, wherein the first mAb comprises the following six CDRs:
i. a light chain CDR 1 comprising an amino acid sequence of SEQ ID NO: 201, or a functional variant thereof;
ii. a light chain CDR 2 comprising an amino acid sequence of SEQ ID NO: 202, or a functional variant thereof;
iii. a light chain CDR 3 comprising an amino acid sequence of SEQ ID NO: 203, or a functional variant thereof;
iv. a heavy chain CDR 4 comprising an amino acid sequence of SEQ ID NO: 204, or a functional variant thereof;
v. a heavy chain CDR 5 comprising an amino acid sequence of SEQ ID NO: 205, or a functional variant thereof;
vi. a heavy chain CDR 6 comprising an amino acid sequence of SEQ ID NO: 206, or a functional variant thereof;
wherein the first mAb binds a S. pneumoniae ST-19A PnPs; and
the second monoclonal antibody (mAb) binds a S. pneumoniae ST-19A PnPs to form a first mAb-antigen-second mAb complex, wherein the second mAb comprises the following six CDRs:
i. a light chain CDR 1 comprising an amino acid sequence of SEQ ID NO: 207, or a functional variant thereof;
ii. a light chain CDR 2 comprising an amino acid sequence of SEQ ID NO: 208, or a functional variant thereof;
iii. a light chain CDR 3 comprising an amino acid sequence of SEQ ID NO: 209, or a functional variant thereof;
iv. a heavy chain CDR 4 comprising an amino acid sequence of SEQ ID NO: 210, or a functional variant thereof;
v. a heavy chain CDR 5 comprising an amino acid sequence of SEQ ID NO: 211, or a functional variant thereof;
vi. a heavy chain CDR 6 comprising an amino acid sequence of SEQ ID NO: 212, or a functional variant thereof;
wherein the second mAb binds a S. pneumoniae ST-19A PnPs.

In another embodiment the invention provides the method above wherein the first monoclonal antibody (mAb) binds a S. pneumoniae ST-19A PnPs to form a first mAb-antigen complex, wherein the first mAb comprises:
i. a variable light chain comprising an amino acid sequence of SEQ ID NO: 213, or a functional variant thereof; and
ii. a variable heavy chain comprising an amino acid sequence of SEQ ID NO: 214, or a functional variant thereof; and
the second monoclonal antibody (mAb) binds a S. pneumoniae ST-19A PnPs to form a first mAb-antigen-second mAb complex, wherein the second mAb comprises:
i. a variable light chain comprising an amino acid sequence of SEQ ID NO: 215, or a functional variant thereof; and
ii. a variable heavy chain comprising an amino acid sequence of SEQ ID NO: 216, or a functional variant thereof.

In another embodiment the invention provides the method above wherein the first monoclonal antibody (mAb) binds a S. pneumoniae ST-19A PnPs to form a first mAb-antigen complex, wherein the first mAb comprises:
i. a full length light chain comprising an amino acid sequence of SEQ ID NO: 217, or a functional variant thereof; and
ii. a full length heavy chain comprising an amino acid sequence of SEQ ID NO: 218, or a functional variant thereof; and
the second monoclonal antibody (mAb) binds a S. pneumoniae ST-19A PnPs to form a first mAb-antigen-second mAb complex, wherein the second mAb comprises:
i. a full length light chain comprising an amino acid sequence of SEQ ID NO: 219, or a functional variant thereof; and
ii. a full length heavy chain comprising an amino acid sequence of SEQ ID NO: 220, or a functional variant thereof.

In another embodiment the invention provides the method above wherein the first monoclonal antibody (mAb) binds a S. pneumoniae ST-19F PnPs to form a first mAb-antigen complex, wherein the first mAb comprises the following six CDRs:
i. a light chain CDR 1 comprising an amino acid sequence of SEQ ID NO: 221, or a functional variant thereof;
ii. a light chain CDR 2 comprising an amino acid sequence of SEQ ID NO: 222, or a functional variant thereof;
iii. a light chain CDR 3 comprising an amino acid sequence of SEQ ID NO: 223, or a functional variant thereof;
iv. a heavy chain CDR 4 comprising an amino acid sequence of SEQ ID NO: 224, or a functional variant thereof;
v. a heavy chain CDR 5 comprising an amino acid sequence of SEQ ID NO: 225, or a functional variant thereof;
vi. a heavy chain CDR 6 comprising an amino acid sequence of SEQ ID NO: 226, or a functional variant thereof;
wherein the first mAb binds a S. pneumoniae ST-19F PnPs; and
the second monoclonal antibody (mAb) binds a S. pneumoniae ST-19F PnPs to form a first mAb-antigen-second mAb complex, wherein the second mAb comprises the following six CDRs:
i. a light chain CDR 1 comprising an amino acid sequence of SEQ ID NO: 227, or a functional variant thereof;
ii. a light chain CDR 2 comprising an amino acid sequence of SEQ ID NO: 228, or a functional variant thereof;
iii. a light chain CDR 3 comprising an amino acid sequence of SEQ ID NO: 229, or a functional variant thereof;
iv. a heavy chain CDR 4 comprising an amino acid sequence of SEQ ID NO: 230, or a functional variant thereof;
v. a heavy chain CDR 5 comprising an amino acid sequence of SEQ ID NO: 231, or a functional variant thereof;
vi. a heavy chain CDR 6 comprising an amino acid sequence of SEQ ID NO: 232, or a functional variant thereof;
wherein the second mAb binds a S. pneumoniae ST-19F PnPs.

In another embodiment the invention provides the method above wherein the first monoclonal antibody (mAb) binds a S. pneumoniae ST-19F PnPs to form a first mAb-antigen complex, wherein the first mAb comprises:
i. a variable light chain comprising an amino acid sequence of SEQ ID NO: 233, or a functional variant thereof; and
ii. a variable heavy chain comprising an amino acid sequence of SEQ ID NO: 234, or a functional variant thereof; and
the second monoclonal antibody (mAb) binds a S. pneumoniae ST-19F PnPs to form a first mAb-antigen-second mAb complex, wherein the second mAb comprises:
i. a variable light chain comprising an amino acid sequence of SEQ ID NO: 235, or a functional variant thereof; and
ii. a variable heavy chain comprising an amino acid sequence of SEQ ID NO: 236, or a functional variant thereof.

In another embodiment the invention provides the method above wherein the first monoclonal antibody (mAb) binds a S. pneumoniae ST-19F PnPs to form a first mAb-antigen complex, wherein the first mAb comprises:
i. a full length light chain comprising an amino acid sequence of SEQ ID NO: 237, or a functional variant thereof; and
ii. a full length heavy chain comprising an amino acid sequence of SEQ ID NO: 238, or a functional variant thereof; and
the second monoclonal antibody (mAb) binds a S. pneumoniae ST-19F PnPs to form a first mAb-antigen-second mAb complex, wherein the second mAb comprises:
i. a full length light chain comprising an amino acid sequence of SEQ ID NO: 239, or a functional variant thereof; and
ii. a full length heavy chain comprising an amino acid sequence of SEQ ID NO: 240, or a functional variant thereof.

In another embodiment the invention provides the method above wherein the first monoclonal antibody (mAb) binds a S. pneumoniae ST-22F PnPs to form a first mAb-antigen complex, wherein the first mAb comprises the following six CDRs:
i. a light chain CDR 1 comprising an amino acid sequence of SEQ ID NO: 241, or a functional variant thereof;
ii. a light chain CDR 2 comprising an amino acid sequence of SEQ ID NO: 242, or a functional variant thereof;
iii. a light chain CDR 3 comprising an amino acid sequence of SEQ ID NO: 243, or a functional variant thereof;
iv. a heavy chain CDR 4 comprising an amino acid sequence of SEQ ID NO: 244, or a functional variant thereof;
v. a heavy chain CDR 5 comprising an amino acid sequence of SEQ ID NO: 245, or a functional variant thereof;
vi. a heavy chain CDR 6 comprising an amino acid sequence of SEQ ID NO: 246, or a functional variant thereof;
wherein the first mAb binds a S. pneumoniae ST-22F PnPs; and
the second monoclonal antibody (mAb) binds a S. pneumoniae ST-22F PnPs to form a first mAb-antigen-second mAb complex, wherein the second mAb comprises the following six CDRs:
i. a light chain CDR 1 comprising an amino acid sequence of SEQ ID NO: 247, or a functional variant thereof;
ii. a light chain CDR 2 comprising an amino acid sequence of SEQ ID NO: 248, or a functional variant thereof;
iii. a light chain CDR 3 comprising an amino acid sequence of SEQ ID NO: 249, or a functional variant thereof;
iv. a heavy chain CDR 4 comprising an amino acid sequence of SEQ ID NO: 250, or a functional variant thereof;
v. a heavy chain CDR 5 comprising an amino acid sequence of SEQ ID NO: 251, or a functional variant thereof;
vi. a heavy chain CDR 6 comprising an amino acid sequence of SEQ ID NO: 252, or a functional variant thereof;
wherein the second mAb binds a S. pneumoniae ST-22F PnPs.

In another embodiment the invention provides the method above wherein the first monoclonal antibody (mAb) binds a S. pneumoniae ST-22F PnPs to form a first mAb-antigen complex, wherein the first mAb comprises:
i. a variable light chain comprising an amino acid sequence of SEQ ID NO: 253, or a functional variant thereof; and
ii. a variable heavy chain comprising an amino acid sequence of SEQ ID NO: 254, or a functional variant thereof; and
the second monoclonal antibody (mAb) binds a S. pneumoniae ST-22F PnPs to form a first mAb-antigen-second mAb complex, wherein the second mAb comprises:
i. a variable light chain comprising an amino acid sequence of SEQ ID NO: 255, or a functional variant thereof; and
ii. a variable heavy chain comprising an amino acid sequence of SEQ ID NO: 256, or a functional variant thereof.

In another embodiment the invention provides the method above wherein the first monoclonal antibody (mAb) binds a S. pneumoniae ST-22F PnPs to form a first mAb-antigen complex, wherein the first mAb comprises:
i. a full length light chain comprising an amino acid sequence of SEQ ID NO: 257, or a functional variant thereof; and
ii. a full length heavy chain comprising an amino acid sequence of SEQ ID NO: 258, or a functional variant thereof; and
the second monoclonal antibody (mAb) binds a S. pneumoniae ST-22F PnPs to form a first mAb-antigen-second mAb complex, wherein the second mAb comprises:
i. a full length light chain comprising an amino acid sequence of SEQ ID NO: 259, or a functional variant thereof; and
ii. a full length heavy chain comprising an amino acid sequence of SEQ ID NO: 260, or a functional variant thereof.

In another embodiment the invention provides the method above wherein the first monoclonal antibody (mAb) binds a S. pneumoniae ST-23F PnPs to form a first mAb-antigen complex, wherein the first mAb comprises the following six CDRs:
i. a light chain CDR 1 comprising an amino acid sequence of SEQ ID NO: 261, or a functional variant thereof;
ii. a light chain CDR 2 comprising an amino acid sequence of SEQ ID NO: 262, or a functional variant thereof;
iii. a light chain CDR 3 comprising an amino acid sequence of SEQ ID NO: 263, or a functional variant thereof;
iv. a heavy chain CDR 4 comprising an amino acid sequence of SEQ ID NO: 264, or a functional variant thereof;
v. a heavy chain CDR 5 comprising an amino acid sequence of SEQ ID NO: 265, or a functional variant thereof;
vi. a heavy chain CDR 6 comprising an amino acid sequence of SEQ ID NO: 266, or a functional variant thereof;
wherein the first mAb binds a S. pneumoniae ST-23F PnPs; and
the second monoclonal antibody (mAb) binds a S. pneumoniae ST-23F PnPs to form a first mAb-antigen-second mAb complex, wherein the second mAb comprises the following six CDRs:
i. a light chain CDR 1 comprising an amino acid sequence of SEQ ID NO: 267, or a functional variant thereof;
ii. a light chain CDR 2 comprising an amino acid sequence of SEQ ID NO: 268, or a functional variant thereof;
iii. a light chain CDR 3 comprising an amino acid sequence of SEQ ID NO: 269, or a functional variant thereof;
iv. a heavy chain CDR 4 comprising an amino acid sequence of SEQ ID NO: 270, or a functional variant thereof;
v. a heavy chain CDR 5 comprising an amino acid sequence of SEQ ID NO: 271, or a functional variant thereof;
vi. a heavy chain CDR 6 comprising an amino acid sequence of SEQ ID NO: 272, or a functional variant thereof;
wherein the second mAb binds a S. pneumoniae ST-23F PnPs.

In another embodiment the invention provides the method above wherein the first monoclonal antibody (mAb) binds a S. pneumoniae ST-23F PnPs to form a first mAb-antigen complex, wherein the first mAb comprises:
i. a variable light chain comprising an amino acid sequence of SEQ ID NO: 273, or a functional variant thereof; and
ii. a variable heavy chain comprising an amino acid sequence of SEQ ID NO: 274, or a functional variant thereof; and
the second monoclonal antibody (mAb) binds a S. pneumoniae ST-23F PnPs to form a first mAb-antigen-second mAb complex, wherein the second mAb comprises:
i. a variable light chain comprising an amino acid sequence of SEQ ID NO: 275, or a functional variant thereof; and
ii. a variable heavy chain comprising an amino acid sequence of SEQ ID NO: 276, or a functional variant thereof.

In another embodiment the invention provides the method above wherein the first monoclonal antibody (mAb) binds a S. pneumoniae ST-23F PnPs to form a first mAb-antigen complex, wherein the first mAb comprises:
i. a full length light chain comprising an amino acid sequence of SEQ ID NO: 277, or a functional variant thereof; and
ii. a full length heavy chain comprising an amino acid sequence of SEQ ID NO: 278, or a functional variant thereof; and
the second monoclonal antibody (mAb) binds a S. pneumoniae ST-23F PnPs to form a first mAb-antigen-second mAb complex, wherein the second mAb comprises:
i. a full length light chain comprising an amino acid sequence of SEQ ID NO: 279, or a functional variant thereof; and
ii. a full length heavy chain comprising an amino acid sequence of SEQ ID NO: 280, or a functional variant thereof.

In another embodiment the invention provides the method above wherein the first monoclonal antibody (mAb) binds a S. pneumoniae ST-33F PnPs to form a first mAb-antigen complex, wherein the first mAb comprises the following six CDRs:
i. a light chain CDR 1 comprising an amino acid sequence of SEQ ID NO: 281, or a functional variant thereof;
ii. a light chain CDR 2 comprising an amino acid sequence of SEQ ID NO: 282, or a functional variant thereof;
iii. a light chain CDR 3 comprising an amino acid sequence of SEQ ID NO: 283, or a functional variant thereof;
iv. a heavy chain CDR 4 comprising an amino acid sequence of SEQ ID NO: 284, or a functional variant thereof;
v. a heavy chain CDR 5 comprising an amino acid sequence of SEQ ID NO: 285, or a functional variant thereof;
vi. a heavy chain CDR 6 comprising an amino acid sequence of SEQ ID NO: 286, or a functional variant thereof;
wherein the first mAb binds a S. pneumoniae ST-33F PnPs; and
the second monoclonal antibody (mAb) binds a S. pneumoniae ST-33F PnPs to form a first mAb-antigen-second mAb complex, wherein the second mAb comprises the following six CDRs:
i. a light chain CDR 1 comprising an amino acid sequence of SEQ ID NO: 287, or a functional variant thereof;
ii. a light chain CDR 2 comprising an amino acid sequence of SEQ ID NO: 288, or a functional variant thereof;
iii. a light chain CDR 3 comprising an amino acid sequence of SEQ ID NO: 289, or a functional variant thereof;
iv. a heavy chain CDR 4 comprising an amino acid sequence of SEQ ID NO: 290, or a functional variant thereof;
v. a heavy chain CDR 5 comprising an amino acid sequence of SEQ ID NO: 291, or a functional variant thereof;
vi. a heavy chain CDR 6 comprising an amino acid sequence of SEQ ID NO: 292, or a functional variant thereof;
wherein the second mAb binds a S. pneumoniae ST-33F PnPs.

In another embodiment the invention provides the method above wherein the first monoclonal antibody (mAb) binds a S. pneumoniae ST-33F PnPs to form a first mAb-antigen complex, wherein the first mAb comprises:
i. a variable light chain comprising an amino acid sequence of SEQ ID NO: 293, or a functional variant thereof; and
ii. a variable heavy chain comprising an amino acid sequence of SEQ ID NO: 294, or a functional variant thereof; and
the second monoclonal antibody (mAb) binds a S. pneumoniae ST-33F PnPs to form a first mAb-antigen-second mAb complex, wherein the second mAb comprises:
i. a variable light chain comprising an amino acid sequence of SEQ ID NO: 295, or a functional variant thereof; and
ii. a variable heavy chain comprising an amino acid sequence of SEQ ID NO: 296, or a functional variant thereof.

In another embodiment the invention provides the method above wherein the first monoclonal antibody (mAb) binds a S. pneumoniae ST-33F PnPs to form a first mAb-antigen complex, wherein the first mAb comprises:
i. a full length light chain comprising an amino acid sequence of SEQ ID NO: 297, or a functional variant thereof; and
ii. a full length heavy chain comprising an amino acid sequence of SEQ ID NO: 298, or a functional variant thereof; and
the second monoclonal antibody (mAb) binds a S. pneumoniae ST-33F PnPs to form a first mAb-antigen-second mAb complex, wherein the second mAb comprises:
i. a full length light chain comprising an amino acid sequence of SEQ ID NO: 299, or a functional variant thereof; and
ii. a full length heavy chain comprising an amino acid sequence of SEQ ID NO: 300, or a functional variant thereof.

In another embodiment the invention provides the method above further comprising contacting the sample with one or more first and second monoclonal antibodies (mAbs), to form one or more first mAb-antigen-second mAb complexes, wherein the one or more first and second monoclonal antibodies (mAbs) bind to one or more S. pneumoniae serotype-specific capsular polysaccharides.

In another embodiment the invention provides the method above wherein the first monoclonal antibody is coupled to a bead.

In another embodiment the invention provides the method above wherein the bead is made of a carboxylated polystyrene material.

In another embodiment the invention provides the method above wherein the bead is a carboxylated polystyrene microsphere.

In another embodiment the invention provides the method above wherein the bead is a magnetic Luminex™ bead.

In another embodiment the invention provides the method above wherein the sample is a human blood sample, a human saliva sample and/or a human urine sample.

In another embodiment the invention provides the method above wherein the sample is a human urine sample.

In another embodiment the invention provides the method above wherein the reporter molecule coupled to the third antibody is phycoerythrin (PE) and binds to the second monoclonal antibody to allow detection and quantitation of the captured PnPs.

In another embodiment the invention provides the method above wherein the reporter molecule coupled to the third antibody is phycoerythrin (PE) and binds to the second monoclonal antibody to allow detection and quantitation of a captured PnPs using a Luminex microfluidics system™.

In an embodiment the invention provides a kit for detecting the presence or absence of one or more S. pneumoniae capsular polysaccharide(s) (PnPs) in a sample, wherein said kit comprises:
a) one or more first monoclonal antibody (mAb), or functional variant thereof, that binds one or more pneumococcal serotype (ST) capsular polysaccharide (PnPs);
b) one or more second monoclonal antibody (mAb), or functional variant thereof, that binds one or more pneumococcal serotype (ST) capsular polysaccharide (PnPs);
c) a third antibody that binds the one or more second monoclonal antibody; and
d) instructions to use said kit.

In another embodiment the invention provides the kit above for detecting the presence or absence of one or more S. pneumoniae capsular polysaccharide(s) (PnPs) in a sample, wherein said kit comprises:
a) one or more first monoclonal antibody (mAb), or functional variant thereof, that binds one or more pneumococcal serotype (ST) capsular polysaccharide (PnPs), said antibody, or functional variant thereof, comprises:
   i) six complementarity determining regions (CDRs) selected from the group consisting of SEQ. ID. NOs.: 1-6, 21-26, 41-46, 61-66, 81-86, 101-106, 121-126, 141-146, 161-166, 181-186, 201-206, 221-226, 241-246, 261-266, and 281-286;
   ii) a variable heavy chain and a variable light chain having an amino acid sequence selected from the group consisting of SEQ ID NOs: 13-14, 33-34, 53-54, 73-74, 93-94, 113-114, 133-134, 153-154, 173-174, 193-194, 213-214, 233-234, 253-254, 273-274, and 293-294; or
   iii) a full length light chain and a full length heavy chain having an amino acid sequence selected from the group consisting of SEQ ID NOs: 17-18, 37-38, 57-58, 77-78, 97-98, 117-118, 137-138, 157-158, 177-178, 197-198, 217-218, 237-238, 257-258, 277-278, and 297-298;
b) one or more second monoclonal antibody (mAb), or functional variant thereof, that binds one or more pneumococcal serotype (ST) capsular polysaccharide (PnPs), said antibody, or functional variant thereof, comprises:
   i) six complementarity determining regions (CDRs) selected from the group consisting of SEQ. ID. NOs.: 7-12, 27-32, 47-52, 67-72, 87-92, 107-112, 127-132, 147-152, 167-172, 187-192, 207-212, 227-232, 247-252, 267-272, and 287-292;
   ii) a variable heavy chain and variable light chain sequence selected from the group consisting of SEQ ID NOs: 15-16, 35-36, 55-56, 75-76, 95-96, 115-116, 135-136, 155-156, 175-176, 195-196, 215-216, 235-236, 255-256, 275-276, and 295-296; or
   iii) a full length light and heavy chain sequence selected from the group consisting of SEQ ID NOs: 19-20, 39-40, 59-60, 79-80, 99-100, 119-120, 139-140, 159-160, 179-180, 199-200, 219-220, 239-240, 259-260, 279-280, and 299-300;
c) a third antibody that binds the second monoclonal antibody; and
d) instructions to use said kit.

In another embodiment the invention provides a monoclonal antibody (mAb), or a functional variant thereof, that specifically binds to a pneumococcal serotype (ST) capsular polysaccharide (PnPs), wherein said mAb comprises:
a) six complementarity determining regions (CDRs) selected from the group consisting of SEQ. ID. NOs.: 1-6, 21-26, 41-46, 61-66, 81-86, 101-106, 121-126, 141-146, 161-166, 181-186, 201-206, 221-226, 241-246, 261-266, 281-286, 7-12, 27-32, 47-52, 67-72, 87-92, 107-112, 127-132, 147-152, 167-172, 187-192, 207-212, 227-232, 247-252, 267-272, and 287-292;
b) a variable heavy chain and a variable light chain having an amino acid sequence selected from the group consisting of SEQ ID NOs: 13-14, 33-34, 53-54, 73-74, 93-94, 113-114, 133-134, 153-154, 173-174, 193-194, 213-214, 233-234, 253-254, 273-274, 293-294, 15-16, 35-36, 55-56, 75-76, 95-96, 115-116, 135-136, 155-156, 175-176, 195-196, 215-216, 235-236, 255-256, 275-276, and 295-296; or
c) a full length light chain and a full length heavy chain having an amino acid sequence selected from the group consisting of SEQ ID NOs: 17-18, 37-38, 57-58, 77-78, 97-98, 117-118, 137-138, 157-158, 177-178, 197-198, 217-218, 237-238, 257-258, 277-278, 297-298, 19-20, 39-40, 59-60, 79-80, 99-100, 119-120, 139-140, 159-160, 179-180, 199-200, 219-220, 239-240, 259-260, 279-280, and 299-300.

In another embodiment the invention provides the monoclonal antibody above, wherein the mAb comprises six CDRs selected from the group consisting of SEQ. ID. NOs.: 1-6, 21-26, 41-46, 61-66, 81-86, 101-106, 121-126, 141-146, 161-166, 181-186, 201-206, 221-226, 241-246, 261-266, and 281-286.

In another embodiment the invention provides the monoclonal antibody above, wherein the mAb comprises six CDRs selected from the group consisting of SEQ. ID. NOs.: 7-12, 27-32, 47-52, 67-72, 87-92, 107-112, 127-132, 147-152, 167-172, 187-192, 207-212, 227-232, 247-252, 267-272, and 287-292.

In another embodiment the invention provides the monoclonal antibody above, wherein the mAb comprises a variable heavy chain and a variable light chain having an amino acid sequence selected from the group consisting of SEQ ID NOs: 13-14, 33-34, 53-54, 73-74, 93-94, 113-114, 133-134, 153-154, 173-174, 193-194, 213-214, 233-234, 253-254, 273-274, and 293-294.

In another embodiment the invention provides the monoclonal antibody above, wherein the mAb comprises a variable heavy chain and a variable light chain having an amino acid sequence selected from the group consisting of SEQ ID NOs: 15-16, 35-36, 55-56, 75-76, 95-96, 115-116, 135-136, 155-156, 175-176, 195-196, 215-216, 235-236, 255-256, 275-276, and 295-296.

In another embodiment the invention provides the monoclonal antibody above, wherein the mAb comprises a full length light chain and a full length heavy chain having an amino acid sequence selected from the group consisting of SEQ ID NOs: 17-18, 37-38, 57-58, 77-78, 97-98, 117-118, 137-138, 157-158, 177-178, 197-198, 217-218, 237-238, 257-258, 277-278, and 297-298.

In another embodiment the invention provides the monoclonal antibody above, wherein the mAb comprises a full length light chain and a full length heavy chain having an amino acid sequence selected from the group consisting of SEQ ID NOs: 19-20, 39-40, 59-60, 79-80, 99-100, 119-120, 139-140, 159-160, 179-180, 199-200, 219-220, 239-240, 259-260, 279-280, and 299-300.

### BRIEF DESCRIPTION OF THE DRAWINGS

FIG. 1: Shows an example pneumococcal antigen detection assay principle and workflow. The capture antibody is the first monoclonal antibody. The detection antibody is the second monoclonal antibody. The tertiary antibody (otherwise known as the “reporter antibody”) is the third antibody (mAb-monoclonal antibody; PE-phycoerythrin; PnPs-pneumococcal polysaccharide; PS-polysaccharide; SSUAD-serotype-specific urine antigen detection).

FIG. 2: Shows assay precision (qualification) by serotype.

FIG. 3. Shows assay specificity (qualification)—geometric mean PnPs concentrations by serotype and sample (concentrations shaded grey are below the LLOQ;

GMC (geometric mean concentration); NS (no spike sample); PC (positive control containing all Vaxneuvance™ pneumococcal polysaccharide serotypes); PnPs (pneumococcal polysaccharide)).

FIG. 4A and FIG. 4B. Shows a consolidated table of parameters from SSUAD validation experiments (the total precision and the ruggedness estimates for types 1 and 5 were established with (without) one extreme concentration; CV (coefficient of variation); Dil (dilution); LOD (limit of detection); LLOQ (lower limit of quantitation); NE (not evaluable); PnPs (pneumococcal polysaccharide); SSUAD (serotype specific urine antigen detection); ULOQ (upper limit of quantitation)).

FIG. 5. Shows assay specificity (validation)—geometric mean PnPs concentrations by serotype and sample (GMC (geometric mean concentration); LLOQ (lower limit of quantitation); NS (no spike sample); PnPs (pneumococcal polysaccharide)).

### DETAILED DESCRIPTION OF THE INVENTION

Despite the availability of robust vaccination options, PD continues to be a major cause of morbidity and mortality. Although there is a database of ST distribution and disease burden for IPD, without sensitive assays, data for non-bacteremic PDs (NBPD) remain scarce. To address this unmet need, we have developed a non-invasive urine antigen assay, SSUAD, a Luminex™-based multiplex assay validated to detect and quantify PnPs of 15 Vaxneuvance™ STs in adult urine.

Early in vitro assays developed to detect PnPs in clinical specimens, (including urine as a potential source of PnPs (Dochez A. R. and Avery O. T. The Elaboration of Specific Soluble Substance by Pneumococcus during Growth. The Journal of experimental medicine. 1917; 26:477-493.), included latex agglutination, counter-current immunoelectrophoresis, and radioimmunoassay (El-Refaie M. and Dulake C. Counter-current immunoelectrophoresis for the diagnosis of pneumococcal chest infection. J Clin Pathol. 1975; 28:801-806; Leinonen M. K. Detection of Pneumococcal Capsular polysaccharide antigens by latex agglutination, counterimmunoelectrophoresis, and radioimmunoassay in middle ear exudates in acute otitis media. Journal of clinical microbiology. 1980; 11:135-140; and O'Neill K. P. et al., Latex agglutination test for diagnosing pneumococcal pneumonia in children in developing countries. BMJ. 1989; 298:1061-1064.). Later, an indirect sandwich enzyme-linked immunosorbent assay (ELISA) was developed to detect serotype-specific PnPs in urine (Leeming J. P. et al., Diagnosis of invasive pneumococcal infection by serotype-specific urinary antigen detection. Journal of clinical microbiology. 2005; 43:4972-4976.). These assays lacked sensitivity, required large specimen volumes, or were low throughput and/or laborious. Luminex xMAP™ technology enabled capturing 100 analytes per well, equating to 100 individual ELISAs. Leveraging this high-throughput system together with ST-specific mAbs, the earliest attempt to detect PnPs in urine was a competitive inhibition assay using the pneumococcal reference serum 89SF (Findlow H. et al., Competitive inhibition flow analysis assay for the non-culture-based detection and scrotyping of pneumococcal capsular polysaccharide. Clin Vaccine Immunol. 2009; 16:222-229.). We have developed and validated a novel 15-plex SSUAD assay to detect and quantify Vaxneuvance™ ST-specific PnPs in adult urine.

The SSUAD assay can quantify 15 Vaxneuvance™ ST-specific PnPs in a single reaction. To ensure assay transferability to a laboratory setting, standardized protocols, reference standards, quality controls, mAbs, and customized data analysis software were developed. The SSUAD assay is accurate, precise, highly sensitive, rugged, and remarkably specific. Some cross-reactivity was observed for ST 19F at very low concentrations (0.485 ng/ml), prompting recalibration of LLOQ to 1.0 ng/mL for this ST. This was expected given previous reports showing cross-reactivity between PnPs-specific antibodies (Yu X. et al., Immunity to cross-reactive serotypes induced by pneumococcal conjugate vaccines in infants. J Infect Dis. 1999; 180:1569-1576 and Cooper D. et al., The 13-valent pneumococcal conjugate vaccine (PCV13) elicits cross-functional opsonophagocytic killing responses in humans to S. pneumoniae serotypes 6C and 7A. Vaccine. 2011; 29:7207-7211.). High SSUAD specificity and sensitivity can be attributed to the use of ST-specific mAbs for both capture and detection, a distinction from previously reported UAD assays that used polyclonal antibodies for detection (Sheppard C. L. et al., Development of a sensitive, multiplexed immunoassay using xMAP beads for detection of serotype-specific S. pneumoniae antigen in urine samples. J Med Microbiol. 2011; 60:49-55.). Use of highly specific detection mAbs may have contributed to high assay sensitivity (0.007-1.0 ng/mL) and throughput with same day assay capability.

Assay precision against Vaxneuvance™ STs in spiking experiments was confirmed during validation with clinical samples. Most importantly, the SSUAD correctly identified Vaxneuvance™ STs in each of the CAPNETZ clinical urine samples known to be culture positive for respective Vaxneuvance™ STs demonstrating 99% (128/129) clinical specificity and sensitivity. This was much higher than 79.3% with an individual assay sensitivity ranging between 20-100%, previously reported for UAD (Sheppard C. L et al., Development of a sensitive, multiplexed immunoassay using xMAP beads for detection of serotype-specific S. pneumoniae antigen in urine samples. J Med Microbiol. 2011; 60:49-55.). Ironically, the heightened specificity may have contributed to SSUAD positivity of some STs in clinical samples in addition to culture-positive STs. Multiple ST-specific PnPs were also reported previously with multiplex UAD (Pride M. W. et al., Validation of an immunodiagnostic assay for detection of 13 S. pneumoniae serotype-specific polysaccharides in human urine. Clin Vaccine Immunol. 2012; 19:1131-1141.). Conversely, the propensity to miss a serotype in a specimen with mixed STs using latex agglutination or Quellung reaction serotyping cannot be ruled out based on low sensitivity and subjectivity.

Urine is a complex matrix and, hence, it is critical to optimize a multiplex indirect PnPs capture assay to ensure minimal interference. Our selectivity data demonstrated recovery of Vaxneuvance™ ST-specific PnPs in urine from different donors with accuracy and minimal matrix interference. In addition, dilutional linearity suggests that sample dilution with reproducible outcomes is possible within the quantifiable range of the SSUAD assay. Assay precision and ruggedness are key characteristics that ensure transferability of SSUAD to other laboratories without further optimization. SSUAD satisfies these criteria with high levels of precision (7.1-14.2%) in the qualification study using spiked samples and 3.2% and 24.9% in the validation study using clinical samples and ruggedness related to bead lots, analyst, and Luminex™ reader. SSUAD utilizes magnetic Luminex™ beads instead of polystyrene beads used previously (Sheppard C. L. et al., Development of a sensitive, multiplexed immunoassay using xMAP beads for detection of serotype-specific S. pneumoniae antigen in urine samples. J Med Microbiol. 2011; 60:49-55.), allowing automation of wash steps and improving assay throughput to support large surveillance or clinical studies.

S. pneumoniae causes a wide range of invasive and non-invasive PDs with potentially fatal outcomes. While IPD is diagnosed using blood culture yielding ST information on the causal organism, identification of STs driving NBPD remains elusive. STs responsible for NBPD may serve as a reservoir that replaces the respiratory niche vacated by vaccine STs after effective vaccinations. A robust assay is needed to interrogate the pneumococcal ST landscape especially in NBPD, and guide development of next-generation vaccines. For example, the increasing prevalence of non-vaccine STs, such as 22F and 33F, may increase the incidence of non-vaccine-type PD in the future (Weinberger D. M. et al., Relating Pneumococcal Carriage Among Children to Disease Rates Among Adults Before and After the Introduction of Conjugate Vaccines. Am J Epidemiol. 2016; 183:1055-1062.). Hence, monitoring changes in STs associated with PD is critical to assess the effectiveness of existing vaccines and direct future vaccine development and deployment.

Studies of pneumococcal ST replacement in CAP are scarce and hampered by low frequency of proactive bacterial testing in clinical settings (Bracken D. C. W. et al., Shift in bacterial etiology from the CAPNETZ cohort in patients with community-acquired pneumonia: data over more than a decade. Infection. 2021; 49:533-537.). The SSUAD assay described in this report will help assess the effectiveness of Vaxneuvance™ against NBPP in ongoing vaccination programs and help monitor the changing burden of PD and the distribution of STs.

Compared with traditional culture assays, the SSUAD assay has advantages. Urine samples are relatively easy to collect and can be stored frozen in batches for later analysis. By contrast, respiratory samples used for bacterial cultures can be difficult to acquire from older or sicker patients (Bracken D. C. W. et al., Shift in bacterial etiology from the CAPNETZ cohort in patients with community-acquired pneumonia: data over more than a decade. Infection. 2021; 49:533-537.). Equipment and reagents required for the SSUAD assay are widely available and well established for routine laboratory purposes. Turnaround time for the SSUAD assay is relatively short, compared with the time required to conduct a Quellung or latex agglutination reaction, and the results from the SSUAD assay are quantifiable. Although a card-based assay is available that detects a common pneumococcal cell wall antigen (BinaxNOW™, Abbott, CA, USA), this test does not measure specific ST(s) present in the sample, which is vital for surveillance studies (Pride M. W. et al., Validation of an immunodiagnostic assay for detection of 13 S. pneumoniae serotype-specific polysaccharides in human urine. Clin Vaccine Immunol. 2012; 19:1131-1141.).

SSUAD is a multiplex assay capable of detecting and quantifying 15 Vaxneuvance™ pneumococcal ST-specific capsular polysaccharide in adult urine. SSUAD is qualified and validated as a highly sensitive and specific assay with acceptable accuracy and precision that can serve as a tool to investigate pneumococcal ST-specific disease burden in NBPP, to assess the impact of pneumococcal vaccines on CAP, and to help inform next-generation vaccine development.

In one aspect, there is provided a method for detecting the presence or absence of a pneumococcal serotype (ST) capsular polysaccharide (PnPs) in a sample, the method comprising:
a) contacting the sample with: a first monoclonal antibody (mAb) that binds the pneumococcal serotype (ST) capsular polysaccharide (PnPs) to form a first mAb-antigen complex;
b) contacting the sample with: a second monoclonal antibody (mAb) that binds the pneumococcal serotype (ST) capsular polysaccharide (PnPs) to form a first mAb-antigen-second mAb complex;
c) contacting the sample with: a third antibody (Ab) that binds the second monoclonal antibody (mAb); wherein the third antibody is detectably tagged with a reporter molecule;
d) detecting the presence or absence of the first mAb-antigen-second mAb complex by detecting the reporter molecule;
wherein the presence of the first mAb-antigen-second mAb complex confirms the presence of a pneumococcal serotype (ST) capsular polysaccharide (PnPs); and
wherein the absence of the first mAb-antigen-second mAb complex is indicative of the absence of a pneumococcal serotype (ST) capsular polysaccharide (PnPs).

In another aspect, the first monoclonal antibody (mAb) binds a S. pneumoniae ST-1 PnPs to form a first mAb-antigen complex, wherein the first mAb comprises the following six CDRs:
i. a light chain CDR 1 comprising an amino acid sequence of SEQ ID NO: 1, or a functional variant thereof;
ii. a light chain CDR 2 comprising an amino acid sequence of SEQ ID NO: 2, or a functional variant thereof;
iii. a light chain CDR 3 comprising an amino acid sequence of SEQ ID NO: 3, or a functional variant thereof;
iv. a heavy chain CDR 4 comprising an amino acid sequence of SEQ ID NO: 4, or a functional variant thereof;
v. a heavy chain CDR 5 comprising an amino acid sequence of SEQ ID NO: 5, or a functional variant thereof;
vi. a heavy chain CDR 6 comprising an amino acid sequence of SEQ ID NO: 6, or a functional variant thereof;
wherein the first mAb binds a S. pneumoniae ST-1 PnPs; and
the second monoclonal antibody (mAb) binds a S. pneumoniae ST-1 PnPs to form a first mAb-antigen-second mAb complex, wherein the second mAb comprises the following six CDRs:
i. a light chain CDR 1 comprising an amino acid sequence of SEQ ID NO: 7, or a functional variant thereof;
ii. a light chain CDR 2 comprising an amino acid sequence of SEQ ID NO: 8, or a functional variant thereof;
iii. a light chain CDR 3 comprising an amino acid sequence of SEQ ID NO: 9, or a functional variant thereof;
iv. a heavy chain CDR 4 comprising an amino acid sequence of SEQ ID NO: 10, or a functional variant thereof;
v. a heavy chain CDR 5 comprising an amino acid sequence of SEQ ID NO: 11, or a functional variant thereof;
vi. a heavy chain CDR 6 comprising an amino acid sequence of SEQ ID NO: 12, or a functional variant thereof;
wherein the second mAb binds a S. pneumoniae ST-1 PnPs.

In another aspect, the first monoclonal antibody (mAb) binds a S. pneumoniae ST-1 PnPs to form a first mAb-antigen complex, wherein the first mAb comprises:
i. a variable light chain comprising an amino acid sequence of SEQ ID NO: 13, or a functional variant thereof; and
ii. a variable heavy chain comprising an amino acid sequence of SEQ ID NO: 14, or a functional variant thereof.

In another aspect, the second monoclonal antibody (mAb) binds a S. pneumoniae ST-1 PnPs to form a first mAb-antigen-second mAb complex, wherein the second mAb comprises:
i. a variable light chain comprising an amino acid sequence of SEQ ID NO: 15, or a functional variant thereof; and
ii. a variable heavy chain comprising an amino acid sequence of SEQ ID NO: 16, or a functional variant thereof.

In another aspect, the first monoclonal antibody (mAb) binds a S. pneumoniae ST-1 PnPs to form a first mAb-antigen complex, wherein the first mAb comprises:
i. a full length light chain comprising an amino acid sequence of SEQ ID NO: 17, or a functional variant thereof; and
ii. a full length heavy chain comprising an amino acid sequence of SEQ ID NO: 18, or a functional variant thereof.

In another aspect, the second monoclonal antibody (mAb) binds a S. pneumoniae ST-1 PnPs to form a first mAb-antigen-second mAb complex, wherein the second mAb comprises:
i. a full length light chain comprising an amino acid sequence of SEQ ID NO: 19, or a functional variant thereof; and
ii. a full length heavy chain comprising an amino acid sequence of SEQ ID NO: 20, or a functional variant thereof.

In another aspect, the first monoclonal antibody (mAb) binds a S. pneumoniae ST-3 PnPs to form a first mAb-antigen complex, wherein the first mAb comprises the following six CDRs:
i. a light chain CDR 1 comprising an amino acid sequence of SEQ ID NO: 21, or a functional variant thereof;
ii. a light chain CDR 2 comprising an amino acid sequence of SEQ ID NO: 22, or a functional variant thereof;
iii. a light chain CDR 3 comprising an amino acid sequence of SEQ ID NO: 23, or a functional variant thereof;
iv. a heavy chain CDR 4 comprising an amino acid sequence of SEQ ID NO: 24, or a functional variant thereof;
v. a heavy chain CDR 5 comprising an amino acid sequence of SEQ ID NO: 25, or a functional variant thereof;
vi. a heavy chain CDR 6 comprising an amino acid sequence of SEQ ID NO: 26, or a functional variant thereof;
wherein the first mAb binds a S. pneumoniae ST-3 PnPs; and
the second monoclonal antibody (mAb) binds a S. pneumoniae ST-3 PnPs to form a first mAb-antigen-second mAb complex, wherein the second mAb comprises the following six CDRs:
i. a light chain CDR 1 comprising an amino acid sequence of SEQ ID NO: 27, or a functional variant thereof;
ii. a light chain CDR 2 comprising an amino acid sequence of SEQ ID NO: 28, or a functional variant thereof;
iii. a light chain CDR 3 comprising an amino acid sequence of SEQ ID NO: 29, or a functional variant thereof;
iv. a heavy chain CDR 4 comprising an amino acid sequence of SEQ ID NO: 30, or a functional variant thereof;
v. a heavy chain CDR 5 comprising an amino acid sequence of SEQ ID NO: 31, or a functional variant thereof;
vi. a heavy chain CDR 6 comprising an amino acid sequence of SEQ ID NO: 32, or a functional variant thereof;
wherein the second mAb binds a S. pneumoniae ST-3 PnPs.

In another aspect, the first monoclonal antibody (mAb) binds a S. pneumoniae ST-3 PnPs to form a first mAb-antigen complex, wherein the first mAb comprises:
i. a variable light chain comprising an amino acid sequence of SEQ ID NO: 33, or a functional variant thereof; and
ii. a variable heavy chain comprising an amino acid sequence of SEQ ID NO: 34, or a functional variant thereof.

In another aspect, the second monoclonal antibody (mAb) binds a S. pneumoniae ST-3 PnPs to form a first mAb-antigen-second mAb complex, wherein the second mAb comprises:
i. a variable light chain comprising an amino acid sequence of SEQ ID NO: 35, or a functional variant thereof; and
ii. a variable heavy chain comprising an amino acid sequence of SEQ ID NO: 36, or a functional variant thereof.

In another aspect, the first monoclonal antibody (mAb) binds a S. pneumoniae ST-3 PnPs to form a first mAb-antigen complex, wherein the first mAb comprises:
i. a full length light chain comprising an amino acid sequence of SEQ ID NO: 37, or a functional variant thereof; and
ii. a full length heavy chain comprising an amino acid sequence of SEQ ID NO: 38, or a functional variant thereof.

In another aspect, the second monoclonal antibody (mAb) binds a S. pneumoniae ST-3 PnPs to form a first mAb-antigen-second mAb complex, wherein the second mAb comprises:
i. a full length light chain comprising an amino acid sequence of SEQ ID NO: 39, or a functional variant thereof; and
ii. a full length heavy chain comprising an amino acid sequence of SEQ ID NO: 40, or a functional variant thereof.

In another aspect, the first monoclonal antibody (mAb) binds a S. pneumoniae ST-4 PnPs to form a first mAb-antigen complex, wherein the first mAb comprises the following six CDRs:
i. a light chain CDR 1 comprising an amino acid sequence of SEQ ID NO: 41, or a functional variant thereof;
ii. a light chain CDR 2 comprising an amino acid sequence of SEQ ID NO: 42, or a functional variant thereof;
iii. a light chain CDR 3 comprising an amino acid sequence of SEQ ID NO: 43, or a functional variant thereof;
iv. a heavy chain CDR 4 comprising an amino acid sequence of SEQ ID NO: 44, or a functional variant thereof;
v. a heavy chain CDR 5 comprising an amino acid sequence of SEQ ID NO: 45, or a functional variant thereof;
vi. a heavy chain CDR 6 comprising an amino acid sequence of SEQ ID NO: 46, or a functional variant thereof;
wherein the first mAb binds a S. pneumoniae ST-4 PnPs; and
the second monoclonal antibody (mAb) binds a S. pneumoniae ST-4 PnPs to form a first mAb-antigen-second mAb complex, wherein the second mAb comprises the following six CDRs:
i. a light chain CDR 1 comprising an amino acid sequence of SEQ ID NO: 47, or a functional variant thereof;
ii. a light chain CDR 2 comprising an amino acid sequence of SEQ ID NO: 48, or a functional variant thereof;
iii. a light chain CDR 3 comprising an amino acid sequence of SEQ ID NO: 49, or a functional variant thereof;
iv. a heavy chain CDR 4 comprising an amino acid sequence of SEQ ID NO: 50, or a functional variant thereof;
v. a heavy chain CDR 5 comprising an amino acid sequence of SEQ ID NO: 51, or a functional variant thereof;
vi. a heavy chain CDR 6 comprising an amino acid sequence of SEQ ID NO: 52, or a functional wherein the second mAb binds a S. pneumoniae ST-4 PnPs.

In another aspect, the first monoclonal antibody (mAb) binds a S. pneumoniae ST-4 PnPs to form a first mAb-antigen complex, wherein the first mAb comprises:
i. a variable light chain comprising an amino acid sequence of SEQ ID NO: 53, or a functional variant thereof; and
ii. a variable heavy chain comprising an amino acid sequence of SEQ ID NO: 54, or a functional variant thereof.

In another aspect, the second monoclonal antibody (mAb) binds a S. pneumoniae ST-4 PnPs to form a first mAb-antigen-second mAb complex, wherein the second mAb comprises:
i. a variable light chain comprising an amino acid sequence of SEQ ID NO: 55, or a functional variant thereof; and
ii. a variable heavy chain comprising an amino acid sequence of SEQ ID NO: 56, or a functional variant thereof.

In another aspect, the first monoclonal antibody (mAb) binds a S. pneumoniae ST-4 PnPs to form a first mAb-antigen complex, wherein the first mAb comprises:
i. a full length light chain comprising an amino acid sequence of SEQ ID NO: 57, or a functional variant thereof; and
ii. a full length heavy chain comprising an amino acid sequence of SEQ ID NO: 58, or a functional variant thereof.

In another aspect, the second monoclonal antibody (mAb) binds a S. pneumoniae ST-4 PnPs to form a first mAb-antigen-second mAb complex, wherein the second mAb comprises:
i. a full length light chain comprising an amino acid sequence of SEQ ID NO: 59, or a functional variant thereof; and
ii. a full length heavy chain comprising an amino acid sequence of SEQ ID NO: 60, or a functional variant thereof.

In another aspect, the first monoclonal antibody (mAb) binds a S. pneumoniae ST-5 PnPs to form a first mAb-antigen complex, wherein the first mAb comprises the following six CDRs:
i. a light chain CDR 1 comprising an amino acid sequence of SEQ ID NO: 61, or a functional variant thereof;
ii. a light chain CDR 2 comprising an amino acid sequence of SEQ ID NO: 62, or a functional variant thereof;
iii. a light chain CDR 3 comprising an amino acid sequence of SEQ ID NO: 63, or a functional variant thereof;
iv. a heavy chain CDR 4 comprising an amino acid sequence of SEQ ID NO: 64, or a functional variant thereof;
v. a heavy chain CDR 5 comprising an amino acid sequence of SEQ ID NO: 65, or a functional variant thereof;
vi. a heavy chain CDR 6 comprising an amino acid sequence of SEQ ID NO: 66, or a functional variant thereof;
wherein the first mAb binds a S. pneumoniae ST-5 PnPs; and
the second monoclonal antibody (mAb) binds a S. pneumoniae ST-5 PnPs to form a first mAb-antigen-second mAb complex, wherein the second mAb comprises the following six CDRs:
i. a light chain CDR 1 comprising an amino acid sequence of SEQ ID NO: 67, or a functional variant thereof;
ii. a light chain CDR 2 comprising an amino acid sequence of SEQ ID NO: 68, or a functional variant thereof;
iii. a light chain CDR 3 comprising an amino acid sequence of SEQ ID NO: 69, or a functional variant thereof;
iv. a heavy chain CDR 4 comprising an amino acid sequence of SEQ ID NO: 70, or a functional variant thereof;
v. a heavy chain CDR 5 comprising an amino acid sequence of SEQ ID NO: 71, or a functional variant thereof;
vi. a heavy chain CDR 6 comprising an amino acid sequence of SEQ ID NO: 72, or a functional variant thereof;
wherein the second mAb binds a S. pneumoniae ST-5 PnPs.

In another aspect, the first monoclonal antibody (mAb) binds a S. pneumoniae ST-5 PnPs to form a first mAb-antigen complex, wherein the first mAb comprises:
i. a variable light chain comprising an amino acid sequence of SEQ ID NO: 73, or a functional variant thereof; and
ii. a variable heavy chain comprising an amino acid sequence of SEQ ID NO: 74, or a functional variant thereof.

In another aspect, the second monoclonal antibody (mAb) binds a S. pneumoniae ST-5 PnPs to form a first mAb-antigen-second mAb complex, wherein the second mAb comprises:
i. a variable light chain comprising an amino acid sequence of SEQ ID NO: 75, or a functional variant thereof; and
ii. a variable heavy chain comprising an amino acid sequence of SEQ ID NO: 76, or a functional variant thereof.

In another aspect, the first monoclonal antibody (mAb) binds a S. pneumoniae ST-5 PnPs to form a first mAb-antigen complex, wherein the first mAb comprises:
i. a full length light chain comprising an amino acid sequence of SEQ ID NO: 77, or a functional variant thereof; and
ii. a full length heavy chain comprising an amino acid sequence of SEQ ID NO: 78, or a functional variant thereof.

In another aspect, the second monoclonal antibody (mAb) binds a S. pneumoniae ST-5 PnPs to form a first mAb-antigen-second mAb complex, wherein the second mAb comprises:
i. a full length light chain comprising an amino acid sequence of SEQ ID NO: 79, or a functional variant thereof; and
ii. a full length heavy chain comprising an amino acid sequence of SEQ ID NO: 80, or a functional variant thereof.

In another aspect, the first monoclonal antibody (mAb) binds a S. pneumoniae ST-6A PnPs to form a first mAb-antigen complex, wherein the first mAb comprises the following six CDRs:
i. a light chain CDR 1 comprising an amino acid sequence of SEQ ID NO: 81, or a functional variant thereof;
ii. a light chain CDR 2 comprising an amino acid sequence of SEQ ID NO: 82, or a functional variant thereof;
iii. a light chain CDR 3 comprising an amino acid sequence of SEQ ID NO: 83, or a functional variant thereof;
iv. a heavy chain CDR 4 comprising an amino acid sequence of SEQ ID NO: 84, or a functional variant thereof;
v. a heavy chain CDR 5 comprising an amino acid sequence of SEQ ID NO: 85, or a functional variant thereof;
vi. a heavy chain CDR 6 comprising an amino acid sequence of SEQ ID NO: 86, or a functional wherein the first mAb binds a S. pneumoniae ST-6A PnPs; and
the second monoclonal antibody (mAb) binds a S. pneumoniae ST-6A PnPs to form a first mAb-antigen-second mAb complex, wherein the second mAb comprises the following six CDRs:
i. a light chain CDR 1 comprising an amino acid sequence of SEQ ID NO: 87, or a functional variant thereof;
ii. a light chain CDR 2 comprising an amino acid sequence of SEQ ID NO: 88, or a functional variant thereof;
iii. a light chain CDR 3 comprising an amino acid sequence of SEQ ID NO: 89, or a functional variant thereof;
iv. a heavy chain CDR 4 comprising an amino acid sequence of SEQ ID NO: 90, or a functional variant thereof;
v. a heavy chain CDR 5 comprising an amino acid sequence of SEQ ID NO: 91, or a functional variant thereof;
vi. a heavy chain CDR 6 comprising an amino acid sequence of SEQ ID NO: 92, or a functional variant thereof;
wherein the second mAb binds a S. pneumoniae ST-6A PnPs.

In another aspect, the first monoclonal antibody (mAb) binds a S. pneumoniae ST-6A PnPs to form a first mAb-antigen complex, wherein the first mAb comprises:
i. a variable light chain comprising an amino acid sequence of SEQ ID NO: 93, or a functional variant thereof; and
ii. a variable heavy chain comprising an amino acid sequence of SEQ ID NO: 94, or a functional variant thereof.

In another aspect, the second monoclonal antibody (mAb) binds a S. pneumoniae ST-6A PnPs to form a first mAb-antigen-second mAb complex, wherein the second mAb comprises:
i. a variable light chain comprising an amino acid sequence of SEQ ID NO: 95, or a functional variant thereof; and
ii. a variable heavy chain comprising an amino acid sequence of SEQ ID NO: 96, or a functional variant thereof.

In another aspect, the first monoclonal antibody (mAb) binds a S. pneumoniae ST-6A PnPs to form a first mAb-antigen complex, wherein the first mAb comprises:
i. a full length light chain comprising an amino acid sequence of SEQ ID NO: 97, or a functional variant thereof; and
ii. a full length heavy chain comprising an amino acid sequence of SEQ ID NO: 98, or a functional variant thereof.

In another aspect, the second monoclonal antibody (mAb) binds a S. pneumoniae ST-6A PnPs to form a first mAb-antigen-second mAb complex, wherein the second mAb comprises:
i. a full length light chain comprising an amino acid sequence of SEQ ID NO: 99, or a functional variant thereof; and
ii. a full length heavy chain comprising an amino acid sequence of SEQ ID NO: 100, or a functional variant thereof.

In another aspect, the first monoclonal antibody (mAb) binds a S. pneumoniae ST-6B PnPs to form a first mAb-antigen complex, wherein the first mAb comprises the following six CDRs:
i. a light chain CDR 1 comprising an amino acid sequence of SEQ ID NO: 101, or a functional variant thereof;
ii. a light chain CDR 2 comprising an amino acid sequence of SEQ ID NO: 102, or a functional variant thereof;
iii. a light chain CDR 3 comprising an amino acid sequence of SEQ ID NO: 103, or a functional variant thereof;
iv. a heavy chain CDR 4 comprising an amino acid sequence of SEQ ID NO: 104, or a functional variant thereof;
v. a heavy chain CDR 5 comprising an amino acid sequence of SEQ ID NO: 105, or a functional variant thereof;
vi. a heavy chain CDR 6 comprising an amino acid sequence of SEQ ID NO: 106, or a functional variant thereof;
wherein the first mAb binds a S. pneumoniae ST-6B PnPs; and
the second monoclonal antibody (mAb) binds a S. pneumoniae ST-6B PnPs to form a first mAb-antigen-second mAb complex, wherein the second mAb comprises the following six CDRs:
i. a light chain CDR 1 comprising an amino acid sequence of SEQ ID NO: 107, or a functional variant thereof;
ii. a light chain CDR 2 comprising an amino acid sequence of SEQ ID NO: 108, or a functional variant thereof;
iii. a light chain CDR 3 comprising an amino acid sequence of SEQ ID NO: 109, or a functional variant thereof;
iv. a heavy chain CDR 4 comprising an amino acid sequence of SEQ ID NO: 110, or a functional variant thereof;
v. a heavy chain CDR 5 comprising an amino acid sequence of SEQ ID NO: 111, or a functional variant thereof;
vi. a heavy chain CDR 6 comprising an amino acid sequence of SEQ ID NO: 112, or a functional variant thereof;
wherein the second mAb binds a S. pneumoniae ST-6B PnPs.

In another aspect, the first monoclonal antibody (mAb) binds a S. pneumoniae ST-6B PnPs to form a first mAb-antigen complex, wherein the first mAb comprises:
i. a variable light chain comprising an amino acid sequence of SEQ ID NO: 113, or a functional variant thereof; and
ii. a variable heavy chain comprising an amino acid sequence of SEQ ID NO: 114, or a functional variant thereof.

In another aspect, the second monoclonal antibody (mAb) binds a S. pneumoniae ST-6B PnPs to form a first mAb-antigen-second mAb complex, wherein the second mAb comprises:
i. a variable light chain comprising an amino acid sequence of SEQ ID NO: 115, or a functional variant thereof; and
ii. a variable heavy chain comprising an amino acid sequence of SEQ ID NO: 116, or a functional variant thereof.

In another aspect, the first monoclonal antibody (mAb) binds a S. pneumoniae ST-6B PnPs to form a first mAb-antigen complex, wherein the first mAb comprises:
i. a full length light chain comprising an amino acid sequence of SEQ ID NO: 117, or a functional variant thereof; and
ii. a full length heavy chain comprising an amino acid sequence of SEQ ID NO: 118, or a functional variant thereof.

In another aspect, the second monoclonal antibody (mAb) binds a S. pneumoniae ST-6B PnPs to form a first mAb-antigen-second mAb complex, wherein the second mAb comprises:
i. a full length light chain comprising an amino acid sequence of SEQ ID NO: 119, or a functional variant thereof; and
ii. a full length heavy chain comprising an amino acid sequence of SEQ ID NO: 120, or a functional variant thereof.

In another aspect, the first monoclonal antibody (mAb) binds a S. pneumoniae ST-7F PnPs to form a first mAb-antigen complex, wherein the first mAb comprises the following six CDRs:
i. a light chain CDR 1 comprising an amino acid sequence of SEQ ID NO: 121, or a functional variant thereof;
ii. a light chain CDR 2 comprising an amino acid sequence of SEQ ID NO: 122, or a functional variant thereof;
iii. a light chain CDR 3 comprising an amino acid sequence of SEQ ID NO: 123, or a functional variant thereof;
iv. a heavy chain CDR 4 comprising an amino acid sequence of SEQ ID NO: 124, or a functional variant thereof;
v. a heavy chain CDR 5 comprising an amino acid sequence of SEQ ID NO: 125, or a functional variant thereof;
vi. a heavy chain CDR 6 comprising an amino acid sequence of SEQ ID NO: 126, or a functional variant thereof;
wherein the first mAb binds a S. pneumoniae ST-7F PnPs; and
the second monoclonal antibody (mAb) binds a S. pneumoniae ST-7F PnPs to form a first mAb-antigen-second mAb complex, wherein the second mAb comprises the following six CDRs:
i. a light chain CDR 1 comprising an amino acid sequence of SEQ ID NO: 127, or a functional variant thereof;
ii. a light chain CDR 2 comprising an amino acid sequence of SEQ ID NO: 128, or a functional variant thereof;
iii. a light chain CDR 3 comprising an amino acid sequence of SEQ ID NO: 129, or a functional variant thereof;
iv. a heavy chain CDR 4 comprising an amino acid sequence of SEQ ID NO: 130, or a functional variant thereof;
v. a heavy chain CDR 5 comprising an amino acid sequence of SEQ ID NO: 131, or a functional variant thereof;
vi. a heavy chain CDR 6 comprising an amino acid sequence of SEQ ID NO: 132, or a functional variant thereof;
wherein the second mAb binds a S. pneumoniae ST-7F PnPs.

In another aspect, the first monoclonal antibody (mAb) binds a S. pneumoniae ST-7F PnPs to form a first mAb-antigen complex, wherein the first mAb comprises:
i. a variable light chain comprising an amino acid sequence of SEQ ID NO: 133, or a functional variant thereof; and
ii. a variable heavy chain comprising an amino acid sequence of SEQ ID NO: 134, or a functional variant thereof.

In another aspect, the second monoclonal antibody (mAb) binds a S. pneumoniae ST-7F PnPs to form a first mAb-antigen-second mAb complex, wherein the second mAb comprises:
i. a variable light chain comprising an amino acid sequence of SEQ ID NO: 135, or a functional variant thereof; and
ii. a variable heavy chain comprising an amino acid sequence of SEQ ID NO: 136, or a functional variant thereof.

In another aspect, the first monoclonal antibody (mAb) binds a S. pneumoniae ST-7F PnPs to form a first mAb-antigen complex, wherein the first mAb comprises:
i. a full length light chain comprising an amino acid sequence of SEQ ID NO: 137, or a functional variant thereof; and
ii. a full length heavy chain comprising an amino acid sequence of SEQ ID NO: 138, or a functional variant thereof.

In another aspect, the second monoclonal antibody (mAb) binds a S. pneumoniae ST-7F PnPs to form a first mAb-antigen-second mAb complex, wherein the second mAb comprises:
i. a full length light chain comprising an amino acid sequence of SEQ ID NO: 139, or a functional variant thereof; and
ii. a full length heavy chain comprising an amino acid sequence of SEQ ID NO: 140, or a functional variant thereof.

In another aspect, the first monoclonal antibody (mAb) binds a S. pneumoniae ST-9V PnPs to form a first mAb-antigen complex, wherein the first mAb comprises the following six CDRs:
i. a light chain CDR 1 comprising an amino acid sequence of SEQ ID NO: 141, or a functional variant thereof;
ii. a light chain CDR 2 comprising an amino acid sequence of SEQ ID NO: 142, or a functional variant thereof;
iii. a light chain CDR 3 comprising an amino acid sequence of SEQ ID NO: 143, or a functional variant thereof;
iv. a heavy chain CDR 4 comprising an amino acid sequence of SEQ ID NO: 144, or a functional variant thereof;
v. a heavy chain CDR 5 comprising an amino acid sequence of SEQ ID NO: 145, or a functional variant thereof;
vi. a heavy chain CDR 6 comprising an amino acid sequence of SEQ ID NO: 146, or a functional variant thereof;
wherein the first mAb binds a S. pneumoniae ST-9V PnPs; and
the second monoclonal antibody (mAb) binds a S. pneumoniae ST-9V PnPs to form a first mAb-antigen-second mAb complex, wherein the second mAb comprises the following six CDRs:
i. a light chain CDR 1 comprising an amino acid sequence of SEQ ID NO: 147, or a functional variant thereof;
ii. a light chain CDR 2 comprising an amino acid sequence of SEQ ID NO: 148, or a functional variant thereof;
iii. a light chain CDR 3 comprising an amino acid sequence of SEQ ID NO: 149, or a functional variant thereof;
iv. a heavy chain CDR 4 comprising an amino acid sequence of SEQ ID NO: 150, or a functional variant thereof;
v. a heavy chain CDR 5 comprising an amino acid sequence of SEQ ID NO: 151, or a functional variant thereof;
vi. a heavy chain CDR 6 comprising an amino acid sequence of SEQ ID NO: 152, or a functional variant thereof;
wherein the second mAb binds a S. pneumoniae ST-9V PnPs.

In another aspect, the first monoclonal antibody (mAb) binds a S. pneumoniae ST-9V PnPs to form a first mAb-antigen complex, wherein the first mAb comprises:
i. a variable light chain comprising an amino acid sequence of SEQ ID NO: 153, or a functional variant thereof; and
ii. a variable heavy chain comprising an amino acid sequence of SEQ ID NO: 154, or a functional variant thereof.

In another aspect, the second monoclonal antibody (mAb) binds a S. pneumoniae ST-9V PnPs to form a first mAb-antigen-second mAb complex, wherein the second mAb comprises:
i. a variable light chain comprising an amino acid sequence of SEQ ID NO: 155, or a functional variant thereof; and
ii. a variable heavy chain comprising an amino acid sequence of SEQ ID NO: 156, or a functional variant thereof.

In another aspect, the first monoclonal antibody (mAb) binds a S. pneumoniae ST-9V PnPs to form a first mAb-antigen complex, wherein the first mAb comprises:
i. a full length light chain comprising an amino acid sequence of SEQ ID NO: 157, or a functional variant thereof; and
ii. a full length heavy chain comprising an amino acid sequence of SEQ ID NO: 158, or a functional variant thereof.

In another aspect, the second monoclonal antibody (mAb) binds a S. pneumoniae ST-9V PnPs to form a first mAb-antigen-second mAb complex, wherein the second mAb comprises:
i. a full length light chain comprising an amino acid sequence of SEQ ID NO: 159, or a functional variant thereof; and
ii. a full length heavy chain comprising an amino acid sequence of SEQ ID NO: 160, or a functional variant thereof.

In another aspect, the first monoclonal antibody (mAb) binds a S. pneumoniae ST-14 PnPs to form a first mAb-antigen complex, wherein the first mAb comprises the following six CDRs:
i. a light chain CDR 1 comprising an amino acid sequence of SEQ ID NO: 161, or a functional variant thereof;
ii. a light chain CDR 2 comprising an amino acid sequence of SEQ ID NO: 162, or a functional variant thereof;
iii. a light chain CDR 3 comprising an amino acid sequence of SEQ ID NO: 163, or a functional variant thereof;
iv. a heavy chain CDR 4 comprising an amino acid sequence of SEQ ID NO: 164, or a functional variant thereof;
v. a heavy chain CDR 5 comprising an amino acid sequence of SEQ ID NO: 165, or a functional variant thereof;
vi. a heavy chain CDR 6 comprising an amino acid sequence of SEQ ID NO: 166, or a functional variant thereof;
wherein the first mAb binds a S. pneumoniae ST-14 PnPs; and
the second monoclonal antibody (mAb) binds a S. pneumoniae ST-14 PnPs to form a first mAb-antigen-second mAb complex, wherein the second mAb comprises the following six CDRs:
i. a light chain CDR 1 comprising an amino acid sequence of SEQ ID NO: 167, or a functional variant thereof;
ii. a light chain CDR 2 comprising an amino acid sequence of SEQ ID NO: 168, or a functional variant thereof;
iii. a light chain CDR 3 comprising an amino acid sequence of SEQ ID NO: 169, or a functional variant thereof;
iv. a heavy chain CDR 4 comprising an amino acid sequence of SEQ ID NO: 170, or a functional variant thereof;
v. a heavy chain CDR 5 comprising an amino acid sequence of SEQ ID NO: 171, or a functional variant thereof;
vi. a heavy chain CDR 6 comprising an amino acid sequence of SEQ ID NO: 172, or a functional variant thereof;
wherein the second mAb binds a S. pneumoniae ST-14 PnPs.

In another aspect, the first monoclonal antibody (mAb) binds a S. pneumoniae ST-14 PnPs to form a first mAb-antigen complex, wherein the first mAb comprises:
i. a variable light chain comprising an amino acid sequence of SEQ ID NO: 173, or a functional variant thereof; and
ii. a variable heavy chain comprising an amino acid sequence of SEQ ID NO: 174, or a functional variant thereof.

In another aspect, the second monoclonal antibody (mAb) binds a S. pneumoniae ST-14 PnPs to form a first mAb-antigen-second mAb complex, wherein the second mAb comprises:
i. a variable light chain comprising an amino acid sequence of SEQ ID NO: 175, or a functional variant thereof; and
ii. a variable heavy chain comprising an amino acid sequence of SEQ ID NO: 176, or a functional variant thereof.

In another aspect, the first monoclonal antibody (mAb) binds a S. pneumoniae ST-14 PnPs to form a first mAb-antigen complex, wherein the first mAb comprises:
i. a full length light chain comprising an amino acid sequence of SEQ ID NO: 177, or a functional variant thereof; and
ii. a full length heavy chain comprising an amino acid sequence of SEQ ID NO: 178, or a functional variant thereof.

In another aspect, the second monoclonal antibody (mAb) binds a S. pneumoniae ST-14 PnPs to form a first mAb-antigen-second mAb complex, wherein the second mAb comprises:
i. a full length light chain comprising an amino acid sequence of SEQ ID NO: 179, or a functional variant thereof; and
ii. a full length heavy chain comprising an amino acid sequence of SEQ ID NO: 180, or a functional variant thereof.

In another aspect, the first monoclonal antibody (mAb) binds a S. pneumoniae ST-18C PnPs to form a first mAb-antigen complex, wherein the first mAb comprises the following six CDRs:
i. a light chain CDR 1 comprising an amino acid sequence of SEQ ID NO: 181, or a functional variant thereof;
ii. a light chain CDR 2 comprising an amino acid sequence of SEQ ID NO: 182, or a functional variant thereof;
iii. a light chain CDR 3 comprising an amino acid sequence of SEQ ID NO: 183, or a functional variant thereof;
iv. a heavy chain CDR 4 comprising an amino acid sequence of SEQ ID NO: 184, or a functional variant thereof;
v. a heavy chain CDR 5 comprising an amino acid sequence of SEQ ID NO: 185, or a functional variant thereof;
vi. a heavy chain CDR 6 comprising an amino acid sequence of SEQ ID NO: 186, or a functional variant thereof;
wherein the first mAb binds a S. pneumoniae ST-18C PnPs; and
the second monoclonal antibody (mAb) binds a S. pneumoniae ST-18C PnPs to form a first mAb-antigen-second mAb complex, wherein the second mAb comprises the following six CDRs:
i. a light chain CDR 1 comprising an amino acid sequence of SEQ ID NO: 187, or a functional variant thereof;
ii. a light chain CDR 2 comprising an amino acid sequence of SEQ ID NO: 188, or a functional variant thereof;
iii. a light chain CDR 3 comprising an amino acid sequence of SEQ ID NO: 189, or a functional variant thereof;
iv. a heavy chain CDR 4 comprising an amino acid sequence of SEQ ID NO: 190, or a functional variant thereof;
v. a heavy chain CDR 5 comprising an amino acid sequence of SEQ ID NO: 191, or a functional variant thereof;
vi. a heavy chain CDR 6 comprising an amino acid sequence of SEQ ID NO: 192, or a functional variant thereof;
wherein the second mAb binds a S. pneumoniae ST-18C PnPs.

In another aspect, the first monoclonal antibody (mAb) binds a S. pneumoniae ST-18C PnPs to form a first mAb-antigen complex, wherein the first mAb comprises:
i. a variable light chain comprising an amino acid sequence of SEQ ID NO: 193, or a functional variant thereof; and
ii. a variable heavy chain comprising an amino acid sequence of SEQ ID NO: 194, or a functional variant thereof.

In another aspect, the second monoclonal antibody (mAb) binds a S. pneumoniae ST-18C PnPs to form a first mAb-antigen-second mAb complex, wherein the second mAb comprises:
i. a variable light chain comprising an amino acid sequence of SEQ ID NO: 195, or a functional variant thereof; and
ii. a variable heavy chain comprising an amino acid sequence of SEQ ID NO: 196, or a functional variant thereof.

In another aspect, the first monoclonal antibody (mAb) binds a S. pneumoniae ST-18C PnPs to form a first mAb-antigen complex, wherein the first mAb comprises:
i. a full length light chain comprising an amino acid sequence of SEQ ID NO: 197, or a functional variant thereof; and
ii. a full length heavy chain comprising an amino acid sequence of SEQ ID NO: 198, or a functional variant thereof.

In another aspect, the second monoclonal antibody (mAb) binds a S. pneumoniae ST-18C PnPs to form a first mAb-antigen-second mAb complex, wherein the second mAb comprises:
i. a full length light chain comprising an amino acid sequence of SEQ ID NO: 199, or a functional variant thereof; and
ii. a full length heavy chain comprising an amino acid sequence of SEQ ID NO: 200, or a functional variant thereof.

In another aspect, the first monoclonal antibody (mAb) binds a S. pneumoniae ST-19A PnPs to form a first mAb-antigen complex, wherein the first mAb comprises the following six CDRs:
i. a light chain CDR 1 comprising an amino acid sequence of SEQ ID NO: 201, or a functional variant thereof;
ii. a light chain CDR 2 comprising an amino acid sequence of SEQ ID NO: 202, or a functional variant thereof;
iii. a light chain CDR 3 comprising an amino acid sequence of SEQ ID NO: 203, or a functional variant thereof;
iv. a heavy chain CDR 4 comprising an amino acid sequence of SEQ ID NO: 204, or a functional variant thereof;
v. a heavy chain CDR 5 comprising an amino acid sequence of SEQ ID NO: 205, or a functional variant thereof;
vi. a heavy chain CDR 6 comprising an amino acid sequence of SEQ ID NO: 206, or a functional variant thereof;
wherein the first mAb binds a S. pneumoniae ST-19A PnPs; and
the second monoclonal antibody (mAb) binds a S. pneumoniae ST-19A PnPs to form a first mAb-antigen-second mAb complex, wherein the second mAb comprises the following six CDRs:
i. a light chain CDR 1 comprising an amino acid sequence of SEQ ID NO: 207, or a functional variant thereof;
ii. a light chain CDR 2 comprising an amino acid sequence of SEQ ID NO: 208, or a functional variant thereof;
iii. a light chain CDR 3 comprising an amino acid sequence of SEQ ID NO: 209, or a functional variant thereof;
iv. a heavy chain CDR 4 comprising an amino acid sequence of SEQ ID NO: 210, or a functional variant thereof;
v. a heavy chain CDR 5 comprising an amino acid sequence of SEQ ID NO: 211, or a functional variant thereof;
vi. a heavy chain CDR 6 comprising an amino acid sequence of SEQ ID NO: 212, or a functional variant thereof;
wherein the second mAb binds a S. pneumoniae ST-19A PnPs.

In another aspect, the first monoclonal antibody (mAb) binds a S. pneumoniae ST-19A PnPs to form a first mAb-antigen complex, wherein the first mAb comprises:
i. a variable light chain comprising an amino acid sequence of SEQ ID NO: 213, or a functional variant thereof; and
ii. a variable heavy chain comprising an amino acid sequence of SEQ ID NO: 214, or a functional variant thereof.

In another aspect, the second monoclonal antibody (mAb) binds a S. pneumoniae ST-19A PnPs to form a first mAb-antigen-second mAb complex, wherein the second mAb comprises:
i. a variable light chain comprising an amino acid sequence of SEQ ID NO: 215, or a functional variant thereof; and
ii. a variable heavy chain comprising an amino acid sequence of SEQ ID NO: 216, or a functional variant thereof.

In another aspect, the first monoclonal antibody (mAb) binds a S. pneumoniae ST-19A PnPs to form a first mAb-antigen complex, wherein the first mAb comprises:
i. a full length light chain comprising an amino acid sequence of SEQ ID NO: 217, or a functional variant thereof; and
ii. a full length heavy chain comprising an amino acid sequence of SEQ ID NO: 218, or a functional variant thereof.

In another aspect, the second monoclonal antibody (mAb) binds a S. pneumoniae ST-19A PnPs to form a first mAb-antigen-second mAb complex, wherein the second mAb comprises:
i. a full length light chain comprising an amino acid sequence of SEQ ID NO: 219, or a functional variant thereof; and
ii. a full length heavy chain comprising an amino acid sequence of SEQ ID NO: 220, or a functional variant thereof.

In another aspect, the first monoclonal antibody (mAb) binds a S. pneumoniae ST-19F PnPs to form a first mAb-antigen complex, wherein the first mAb comprises the following six CDRs:
i. a light chain CDR 1 comprising an amino acid sequence of SEQ ID NO: 221, or a functional variant thereof;
ii. a light chain CDR 2 comprising an amino acid sequence of SEQ ID NO: 222, or a functional variant thereof;
iii. a light chain CDR 3 comprising an amino acid sequence of SEQ ID NO: 223, or a functional variant thereof;
iv. a heavy chain CDR 4 comprising an amino acid sequence of SEQ ID NO: 224, or a functional variant thereof;
v. a heavy chain CDR 5 comprising an amino acid sequence of SEQ ID NO: 225, or a functional variant thereof;
vi. a heavy chain CDR 6 comprising an amino acid sequence of SEQ ID NO: 226, or a functional variant thereof;
wherein the first mAb binds a S. pneumoniae ST-19F PnPs; and
the second monoclonal antibody (mAb) binds a S. pneumoniae ST-19F PnPs to form a first mAb-antigen-second mAb complex, wherein the second mAb comprises the following six CDRs:
i. a light chain CDR 1 comprising an amino acid sequence of SEQ ID NO: 227, or a functional variant thereof;
ii. a light chain CDR 2 comprising an amino acid sequence of SEQ ID NO: 228, or a functional variant thereof;
iii. a light chain CDR 3 comprising an amino acid sequence of SEQ ID NO: 229, or a functional variant thereof;
iv. a heavy chain CDR 4 comprising an amino acid sequence of SEQ ID NO: 230, or a functional variant thereof;
v. a heavy chain CDR 5 comprising an amino acid sequence of SEQ ID NO: 231, or a functional variant thereof;
vi. a heavy chain CDR 6 comprising an amino acid sequence of SEQ ID NO: 232, or a functional variant thereof;
wherein the second mAb binds a S. pneumoniae ST-19F PnPs.

In another aspect, the first monoclonal antibody (mAb) binds a S. pneumoniae ST-19F PnPs to form a first mAb-antigen complex, wherein the first mAb comprises:
i. a variable light chain comprising an amino acid sequence of SEQ ID NO: 233, or a functional variant thereof; and
ii. a variable heavy chain comprising an amino acid sequence of SEQ ID NO: 234, or a functional variant thereof.

In another aspect, the second monoclonal antibody (mAb) binds a S. pneumoniae ST-19F PnPs to form a first mAb-antigen-second mAb complex, wherein the second mAb comprises:
i. a variable light chain comprising an amino acid sequence of SEQ ID NO: 235, or a functional variant thereof; and
ii. a variable heavy chain comprising an amino acid sequence of SEQ ID NO: 236, or a functional variant thereof.

In another aspect, the first monoclonal antibody (mAb) binds a S. pneumoniae ST-19F PnPs to form a first mAb-antigen complex, wherein the first mAb comprises:
i. a full length light chain comprising an amino acid sequence of SEQ ID NO: 237, or a functional variant thereof; and
ii. a full length heavy chain comprising an amino acid sequence of SEQ ID NO: 238, or a functional variant thereof.

In another aspect, the second monoclonal antibody (mAb) binds a S. pneumoniae ST-19F PnPs to form a first mAb-antigen-second mAb complex, wherein the second mAb comprises:
i. a full length light chain comprising an amino acid sequence of SEQ ID NO: 239, or a functional variant thereof; and
ii. a full length heavy chain comprising an amino acid sequence of SEQ ID NO: 240, or a functional variant thereof.

In another aspect, the first monoclonal antibody (mAb) binds a S. pneumoniae ST-22F PnPs to form a first mAb-antigen complex, wherein the first mAb comprises the following six CDRs:
i. a light chain CDR 1 comprising an amino acid sequence of SEQ ID NO: 241, or a functional variant thereof;
ii. a light chain CDR 2 comprising an amino acid sequence of SEQ ID NO: 242, or a functional variant thereof;
iii. a light chain CDR 3 comprising an amino acid sequence of SEQ ID NO: 243, or a functional variant thereof;
iv. a heavy chain CDR 4 comprising an amino acid sequence of SEQ ID NO: 244, or a functional variant thereof;
v. a heavy chain CDR 5 comprising an amino acid sequence of SEQ ID NO: 245, or a functional variant thereof;
vi. a heavy chain CDR 6 comprising an amino acid sequence of SEQ ID NO: 246, or a functional variant thereof;
wherein the first mAb binds a S. pneumoniae ST-22F PnPs; and
the second monoclonal antibody (mAb) binds a S. pneumoniae ST-22F PnPs to form a first mAb-antigen-second mAb complex, wherein the second mAb comprises the following six CDRs:
i. a light chain CDR 1 comprising an amino acid sequence of SEQ ID NO: 247, or a functional variant thereof;
ii. a light chain CDR 2 comprising an amino acid sequence of SEQ ID NO: 248, or a functional variant thereof;
iii. a light chain CDR 3 comprising an amino acid sequence of SEQ ID NO: 249, or a functional variant thereof;
iv. a heavy chain CDR 4 comprising an amino acid sequence of SEQ ID NO: 250, or a functional variant thereof;
v. a heavy chain CDR 5 comprising an amino acid sequence of SEQ ID NO: 251, or a functional variant thereof;
vi. a heavy chain CDR 6 comprising an amino acid sequence of SEQ ID NO: 252, or a functional variant thereof;
wherein the second mAb binds a S. pneumoniae ST-22F PnPs.

In another aspect, the first monoclonal antibody (mAb) binds a S. pneumoniae ST-22F PnPs to form a first mAb-antigen complex, wherein the first mAb comprises:
i. a variable light chain comprising an amino acid sequence of SEQ ID NO: 253, or a functional variant thereof; and
ii. a variable heavy chain comprising an amino acid sequence of SEQ ID NO: 254, or a functional variant thereof.

In another aspect, the second monoclonal antibody (mAb) binds a S. pneumoniae ST-22F PnPs to form a first mAb-antigen-second mAb complex, wherein the second mAb comprises:
i. a variable light chain comprising an amino acid sequence of SEQ ID NO: 255, or a functional variant thereof; and
ii. a variable heavy chain comprising an amino acid sequence of SEQ ID NO: 256, or a functional variant thereof.

In another aspect, the first monoclonal antibody (mAb) binds a S. pneumoniae ST-22F PnPs to form a first mAb-antigen complex, wherein the first mAb comprises:
i. a full length light chain comprising an amino acid sequence of SEQ ID NO: 257, or a functional variant thereof; and
ii. a full length heavy chain comprising an amino acid sequence of SEQ ID NO: 258, or a functional variant thereof.

In another aspect, the second monoclonal antibody (mAb) binds a S. pneumoniae ST-22F PnPs to form a first mAb-antigen-second mAb complex, wherein the second mAb comprises:
i. a full length light chain comprising an amino acid sequence of SEQ ID NO: 259, or a functional variant thereof; and
ii. a full length heavy chain comprising an amino acid sequence of SEQ ID NO: 260, or a functional variant thereof.

In another aspect, the first monoclonal antibody (mAb) binds a S. pneumoniae ST-23F PnPs to form a first mAb-antigen complex, wherein the first mAb comprises the following six CDRs:
i. a light chain CDR 1 comprising an amino acid sequence of SEQ ID NO: 261, or a functional variant thereof;
ii. a light chain CDR 2 comprising an amino acid sequence of SEQ ID NO: 262, or a functional variant thereof;
iii. a light chain CDR 3 comprising an amino acid sequence of SEQ ID NO: 263, or a functional variant thereof;
iv. a heavy chain CDR 4 comprising an amino acid sequence of SEQ ID NO: 264, or a functional variant thereof;
v. a heavy chain CDR 5 comprising an amino acid sequence of SEQ ID NO: 265, or a functional variant thereof;
vi. a heavy chain CDR 6 comprising an amino acid sequence of SEQ ID NO: 266, or a functional variant thereof;
wherein the first mAb binds a S. pneumoniae ST-23F PnPs; and
the second monoclonal antibody (mAb) binds a S. pneumoniae ST-23F PnPs to form a first mAb-antigen-second mAb complex, wherein the second mAb comprises the following six CDRs:
i. a light chain CDR 1 comprising an amino acid sequence of SEQ ID NO: 267, or a functional variant thereof;
ii. a light chain CDR 2 comprising an amino acid sequence of SEQ ID NO: 268, or a functional variant thereof;
iii. a light chain CDR 3 comprising an amino acid sequence of SEQ ID NO: 269, or a functional variant thereof;
iv. a heavy chain CDR 4 comprising an amino acid sequence of SEQ ID NO: 270, or a functional variant thereof;
v. a heavy chain CDR 5 comprising an amino acid sequence of SEQ ID NO: 271, or a functional variant thereof;
vi. a heavy chain CDR 6 comprising an amino acid sequence of SEQ ID NO: 272, or a functional variant thereof;
wherein the second mAb binds a S. pneumoniae ST-23F PnPs.

In another aspect, the first monoclonal antibody (mAb) binds a S. pneumoniae ST-23F PnPs to form a first mAb-antigen complex, wherein the first mAb comprises:
i. a variable light chain comprising an amino acid sequence of SEQ ID NO: 273, or a functional variant thereof; and
ii. a variable heavy chain comprising an amino acid sequence of SEQ ID NO: 274, or a functional variant thereof.

In another aspect, the second monoclonal antibody (mAb) binds a S. pneumoniae ST-23F PnPs to form a first mAb-antigen-second mAb complex, wherein the second mAb comprises:
i. a variable light chain comprising an amino acid sequence of SEQ ID NO: 275, or a functional variant thereof; and
ii. a variable heavy chain comprising an amino acid sequence of SEQ ID NO: 276, or a functional variant thereof.

In another aspect, the first monoclonal antibody (mAb) binds a S. pneumoniae ST-23F PnPs to form a first mAb-antigen complex, wherein the first mAb comprises:
i. a full length light chain comprising an amino acid sequence of SEQ ID NO: 277, or a functional variant thereof; and
ii. a full length heavy chain comprising an amino acid sequence of SEQ ID NO: 278, or a functional variant thereof.

In another aspect, the second monoclonal antibody (mAb) binds a S. pneumoniae ST-23F PnPs to form a first mAb-antigen-second mAb complex, wherein the second mAb comprises:
i. a full length light chain comprising an amino acid sequence of SEQ ID NO: 279, or a functional variant thereof; and
ii. a full length heavy chain comprising an amino acid sequence of SEQ ID NO: 280, or a functional variant thereof.

In another aspect, the first monoclonal antibody (mAb) binds a S. pneumoniae ST-33F PnPs to form a first mAb-antigen complex, wherein the first mAb comprises the following six CDRs:
i. a light chain CDR 1 comprising an amino acid sequence of SEQ ID NO: 281, or a functional variant thereof;
ii. a light chain CDR 2 comprising an amino acid sequence of SEQ ID NO: 282, or a functional variant thereof;
iii. a light chain CDR 3 comprising an amino acid sequence of SEQ ID NO: 283, or a functional variant thereof;
iv. a heavy chain CDR 4 comprising an amino acid sequence of SEQ ID NO: 284, or a functional variant thereof;
v. a heavy chain CDR 5 comprising an amino acid sequence of SEQ ID NO: 285, or a functional variant thereof;
vi. a heavy chain CDR 6 comprising an amino acid sequence of SEQ ID NO: 286, or a functional variant thereof;
wherein the first mAb binds a S. pneumoniae ST-33F PnPs; and
the second monoclonal antibody (mAb) binds a S. pneumoniae ST-33F PnPs to form a first mAb-antigen-second mAb complex, wherein the second mAb comprises the following six CDRs:
i. a light chain CDR 1 comprising an amino acid sequence of SEQ ID NO: 287, or a functional variant thereof;
ii. a light chain CDR 2 comprising an amino acid sequence of SEQ ID NO: 288, or a functional variant thereof;
iii. a light chain CDR 3 comprising an amino acid sequence of SEQ ID NO: 289, or a functional variant thereof;
iv. a heavy chain CDR 4 comprising an amino acid sequence of SEQ ID NO: 290, or a functional variant thereof;
v. a heavy chain CDR 5 comprising an amino acid sequence of SEQ ID NO: 291, or a functional variant thereof;
vi. a heavy chain CDR 6 comprising an amino acid sequence of SEQ ID NO: 292, or a functional variant thereof;
wherein the second mAb binds a S. pneumoniae ST-33F PnPs.

In another aspect, the first monoclonal antibody (mAb) binds a S. pneumoniae ST-33F PnPs to form a first mAb-antigen complex, wherein the first mAb comprises:
i. a variable light chain comprising an amino acid sequence of SEQ ID NO: 293, or a functional variant thereof; and
ii. a variable heavy chain comprising an amino acid sequence of SEQ ID NO: 294, or a functional variant thereof.

In another aspect, the second monoclonal antibody (mAb) binds a S. pneumoniae ST-33F PnPs to form a first mAb-antigen-second mAb complex, wherein the second mAb comprises:
i. a variable light chain comprising an amino acid sequence of SEQ ID NO: 295, or a functional variant thereof; and
ii. a variable heavy chain comprising an amino acid sequence of SEQ ID NO: 296, or a functional variant thereof.

In another aspect, the first monoclonal antibody (mAb) binds a S. pneumoniae ST-33F PnPs to form a first mAb-antigen complex, wherein the first mAb comprises:
i. a full length light chain comprising an amino acid sequence of SEQ ID NO: 297, or a functional variant thereof; and
ii. a full length heavy chain comprising an amino acid sequence of SEQ ID NO: 298, or a functional variant thereof.

In another aspect, the second monoclonal antibody (mAb) binds a S. pneumoniae ST-33F PnPs to form a first mAb-antigen-second mAb complex, wherein the second mAb comprises:
i. a full length light chain comprising an amino acid sequence of SEQ ID NO: 299, or a functional variant thereof; and
ii. a full length heavy chain comprising an amino acid sequence of SEQ ID NO: 300, or a functional variant thereof.

In another aspect, the first monoclonal antibody (mAb) is used as a primary capture antibody. In another embodiment, the first monoclonal antibody (mAb) is coupled to a bead. In another embodiment, the first monoclonal antibody (mAb) is coupled to a bead using diimide conjugation chemistry. In another embodiment, the bead is a Luminex™ bead.

In another aspect, the second monoclonal (mAb) is used as a secondary reagent (a secondary mAb, otherwise known as a detection mAb).

In another aspect, the third antibody (Ab) is a reporter antibody. Reporter antibodies to be used in the methods of the present invention can be labeled with any molecule capable of being detected by a flow cytometer or other detection instrument, hereinafter “reporter molecule”. A reporter molecule, therefore, should be chosen which emits light within the range detectable by the instrument. Instruments for use in the present invention comprise a method of excitation, such as a laser, which have a known excitation wavelength that dictates the necessary emission wavelength of the reporter molecule. For example, the LUMINEX 100™ (Luminex Corp., Austin, TX) detection instrument comprises an argon laser, which has an excitation wavelength of 532 nm. Based on this excitation wavelength, one of skill in the art choosing to use the LUMINEX 1001 for use in the present invention, must choose a reporter molecule which emits light at or near 575 nm. Varying the method of excitation, therefore, will allow the use of a greater variety of reporter molecules.

In one embodiment of the invention, the reporter antibody is labeled with a fluorescent reporter molecule. One of skill in the art will recognize that any fluorescent molecule capable of being detected and/or quantified by the detection instrument can be used as the reporter molecule to label the reporter antibody for use in the methods of the invention. As discussed above, the means of excitation and the detection means of the detection instrument will dictate the choice of available reporter molecules. Reporter molecules may include, but are not limited to the following: fluorescein isothiocyanate (FITC), phycoerythrin (PE), cytofluor tangerine, Alexa™ 532 and Alexa™ 546 (Molecular Probes, Eugene, OR), cyanine 3 (Cy3), cyanine 5 (Cy5), cyanine 5.5 (Cy5.5; Amersham Pharmacia Biotech, Piscataway, NJ), lissamine rhodamine B, tetramethylrhodamine isothiocyanate (TRITC), sulforhodamine B, BODIPY-TMR-X (Molecular Probes), PBXL-1 (Martek Biosciences, Columbia, MD), Texas red-avidin (Molecular Probes), streptavidin, C-phycocyanin, R-phycocyaninc II, allophycocyanins (APC) such as APC-B, peridinin chlorophyll protein (PerCP), cascade blue, coumarin. Other fluorescent reporters that can be used in conjunction with the methods of the present invention are well known in the art (see, e.g., Shapiro, H. M., Practical Flow Cytometry, Third edition. New York: Wiley-Liss, 1995, which is herein incorporated by reference).

In an embodiment, the third antibody (Ab) is coupled to a fluorescent reporter molecule and binds to the secondary mAb (the second mAb) to allow detection and quantitation of the captured PnPs. In a further embodiment, the third antibody (Ab) is coupled to phycoerythrin (PE) and binds to the secondary mAb (the second mAb) to allow detection and quantitation of the captured PnPs. In another embodiment, the third antibody (Ab) is coupled to phycocrythrin (PE) and binds to a secondary mAb (the second mAb) to allow detection and quantitation of a captured analyte using the Luminex microfluidics system™. In another embodiment the third antibody is monoclonal or polyclonal. In a further embodiment, the third antibody is polyclonal.

In another aspect, the third antibody (Ab) is a commercial antibody (for example, Jackson ImmunoResearch, PA. Cat #111-117-008; trade name: R-phycoerythrin (PE) conjugated affinipure Fab fragment Goat anti-rabbit IgG, Fc fragment specific; which is an anti-human IgG specific pAb raised in Goat, purified and conjugated with PE and provided as freeze-dried powder.

In an embodiment, the antibodies of the instant invention comprise the following complementarity determining regions (CDRs), variable heavy chains, variable light chains, full length heavy chains and/or full length light chains:

| NAME | A.A. Sequence | SEQ. ID. NO. |
|---|---|---|
| ST-1 First Monoclonal | GLTSGSVSTSHYPS | SEQ. ID. NO.: 1 |
| Antibody (Human) | | |
| Light Chain CDR 1 | | |
| ST-1 First Monoclonal | RTNTRSS | SEQ. ID. NO.: 2 |
| Antibody (Human) | | |
| Light Chain CDR 2 | | |
| ST-1 First Monoclonal | VLFMGSGTWV | SEQ. ID. NO.: 3 |
| Antibody (Human) | | |
| Light Chain CDR 3 | | |
| ST-1 First Monoclonal | GFTVSRSYMS | SEQ. ID. NO.: 4 |
| Antibody (Human) | | |
| Heavy Chain CDR 1 | | |
| ST-1 First Monoclonal | IIYRDGTTYYADSVKG | SEQ. ID. NO.: 5 |
| Antibody | | |
| (Human) | | |
| Heavy Chain CDR 2 | | |
| ST-1 First Monoclonal | EVDYAFDP | SEQ. ID. NO.: 6 |
| Antibody | | |
| (Human) | | |
| Heavy Chain CDR 3 | | |
| ST-1 Second | GLTSGSVSTSHYPS | SEQ. ID. NO.: 7 |
| Monoclonal Antibody | | |
| (Rabbit) | | |
| Light Chain CDR 1 | | |
| ST-1 Second | RTNTRSS | SEQ. ID. NO.: 8 |
| Monoclonal Antibody | | |
| (Rabbit) | | |
| Light Chain CDR 2 | | |
| ST-1 Second | VLFMGSGTWV | SEQ. ID. NO.: 9 |
| Monoclonal Antibody | | |
| (Rabbit) | | |
| Light Chain CDR 3 | | |
| ST-1 Second | GFTVSRSYMS | SEQ. ID. NO.: 10 |
| Monoclonal Antibody | | |
| (Rabbit) | | |
| Heavy Chain CDR 1 | | |
| ST-1 Second | IIYRDGTTYYADSVKG | SEQ. ID. NO.: 11 |
| Monoclonal Antibody | | |
| (Rabbit) | | |
| Heavy Chain CDR 2 | | |
| ST-1 Second | EVDYAFDP | SEQ. ID. NO.: 12 |
| Monoclonal Antibody | | |
| (Rabbit) | | |
| Heavy Chain CDR 3 | | |
| ST-1 First Monoclonal | QTVVTQEPSFSVSPGGTVTLTCGLTSGSVSTSHYPSWYQ | SEQ. ID. NO.: 13 |
| Antibody | QTPGQAPRTLIYRTNTRSSGVPDRESGSILGNKAALTITG | |
| (Human) | AQADDESDYYCVLFMGSGTWVFGGGTKLTVL | |
| Variable Light Chain | | |
| ST-1 First Monoclonal | EVQLVESGGGLIQPGGSLRLSCAASGFTVSRSYMSWVR | SEQ. ID. NO.: 14 |
| Antibody | QAPGKGLEWVSIIYRDGTTYYADSVKGRFTISRDNSKN | |
| (Human) | TLYLQMNSLRADDTAVYYCAREVDYAFDPWGQGTLV | |
| Variable Heavy Chain | TVSS | |
| ST-1 Second | QTVVTQEPSFSVSPGGTVTLTCGLTSGSVSTSHYPSWYQ | SEQ. ID. NO.: 15 |
| Monoclonal Antibody | QTPGQAPRTLIYRTNTRSSGVPDRESGSILGNKAALTITG | |
| (Rabbit) | AQADDESDYYCVLFMGSGTWVFGGGTKLTVL | |
| Variable Light Chain | | |
| ST-1 Second | EVQLVESGGGLIQPGGSLRLSCAASGFTVSRSYMSWVR | SEQ. ID. NO.: 16 |
| Monoclonal Antibody | QAPGKGLEWVSIIYRDGTTYYADSVKGRFTISRDNSKN | |
| (Rabbit) | TLYLQMNSLRADDTAVYYCAREVDYAFDPWGQGTLV | |
| Variable Heavy Chain | TVSS | |
| ST-1 First Monoclonal | QTVVTQEPSFSVSPGGTVTLTCGLTSGSVSTSHYPSWYQ | SEQ. ID. NO.: 17 |
| Antibody | QTPGQAPRTLIYRTNTRSSGVPDRFSGSILGNKAALTITG | |
| (Human) | AQADDESDYYCVLFMGSGTWVFGGGTKLTVLGQPKA | |
| Full Length Light Chain | APSVTLFPPSSEELQANKATLVCLISDFYPGAVTVAWKA | |
| | DSSPVKAGVETTTPSKQSNNKYAASSYLSLTPEQWKSH | |
| | RSYSCQVTHEGSTVEKTVAPTECS | |
| ST-1 First Monoclonal | EVQLVESGGGLIQPGGSLRLSCAASGFTVSRSYMSWVR | SEQ. ID. NO.: 18 |
| Antibody | QAPGKGLEWVSIIYRDGTTYYADSVKGRFTISRDNSKN | |
| (Human) | TLYLQMNSLRADDTAVYYCAREVDYAFDPWGQGTLV | |
| Full Length Heavy | TVSSASTKGPSVFPLAPSSKSTSGGTAALGCLVKDYFPE | |
| Chain | PVTVSWNSGALTSGVHTFPAVLQSSGLYSLSSVVTVPSS | |
| | SLGTQTYICNVNHKPSNTKVDKKVEPKSCDKTHTCPPC | |
| | PAPELLGGPSVFLFPPKPKDTLMISRTPEVTCVVVDVSH | |
| | EDPEVKFNWYVDGVEVHNAKTKPREEQYNSTYRVVSV | |
| | LTVLHQDWLNGKEYKCKVSNKALPAPIEKTISKAKGQP | |
| | REPQVYTLPPSRDELTKNQVSLTCLVKGFYPSDIAVEWE | |
| | SNGQPENNYKTTPPVLDSDGSFFLYSKLTVDKSRWQQG | |
| | NVFSCSVMHEALHNHYTQKSLSLSPGK | |
| ST-1 Second | QTVVTQEPSFSVSPGGTVTLTCGLTSGSVSTSHYPSWYQ | SEQ. ID. NO.: 19 |
| Monoclonal Antibody | QTPGQAPRTLIYRTNTRSSGVPDRFSGSILGNKAALTITG | |
| (Rabbit) | AQADDESDYYCVLFMGSGTWVFGGGTKLTVLGQPAVT | |
| Full Length Light Chain | PSVILFPPSSEELKDNKATLVCLISDFYPRTVKVNWKAD | |
| | GNSVTQGVDTTQPSKQSNNKYAASSFLHLTANQWKSY | |
| | QSVTCQVTHEGHTVEKSLAPAECS | |
| ST-1 Second | EVQLVESGGGLIQPGGSLRLSCAASGFTVSRSYMSWVR | SEQ. ID. NO.: 20 |
| Monoclonal Antibody | QAPGKGLEWVSIIYRDGTTYYADSVKGRFTISRDNSKN | |
| (Rabbit) | TLYLQMNSLRADDTAVYYCAREVDYAFDPWGQGTLV | |
| Full Length Heavy | TVSSGQPKAPSVFPLAPCCGDTPSSTVTLGCLVKGYLPE | |
| Chain | PVTVTWNSGTLTNGVRTFPSVRQSSGLYSLSSVVSVTSS | |
| | SQPVTCNVAHPATNTKVDKTVAPSTCSKPTCPPPELLGG | |
| | PSVFIFPPKPKDTLMISRTPEVTCVVVDVSQDDPEVQFT | |
| | WYINNEQVRTARPPLREQQFNSTIRVVSTLPIAHQDWLR | |
| | GKEFKCKVHNKALPAPIEKTISKARGQPLEPKVYTMGPP | |
| | REELSSRSVSLTCMINGFYPSDISVEWEKNGKAEDNYKT | |
| | TPAVLDSDGSYFLYSKLSVPTSEWQRGDVFTCSVMHEA | |
| | LHNHYTQKSISRSPGK | |
| ST-3 First Monoclonal | QASQSIGSSLA | SEQ. ID. NO.: 21 |
| Antibody (Human) | | |
| Light Chain CDR 1 | | |
| ST-3 First Monoclonal | QASKLAS | SEQ. ID. NO.: 22 |
| Antibody | | |
| (Human) | | |
| Light Chain CDR 2 | | |
| ST-3 First Monoclonal | QCTGNGGDFIGA | SEQ. ID. NO.: 23 |
| Antibody | | |
| (Human) | | |
| Light Chain CDR 3 | | |
| ST-3 First Monoclonal | GFSLSSYYVR | SEQ. ID. NO.: 24 |
| Antibody | | |
| (Human) | | |
| Heavy Chain CDR 1 | | |
| ST-3 First Monoclonal | IISDSGSTYYASWAKG | SEQ. ID. NO.: 25 |
| Antibody | | |
| (Human) | | |
| Heavy Chain CDR 2 | | |
| ST-3 First Monoclonal | GSGYTIPTDL | SEQ. ID. NO.: 26 |
| Antibody | | |
| (Human) | | |
| Heavy Chain CDR 3 | | |
| ST-3 Second | QASQSIGSSLA | SEQ. ID. NO.: 27 |
| Monoclonal Antibody | | |
| (Rabbit) | | |
| Light Chain CDR 1 | | |
| ST-3 Second | QASKLAS | SEQ. ID. NO.: 28 |
| Monoclonal Antibody | | |
| (Rabbit) | | |
| Light Chain CDR 2 | | |
| ST-3 Second | QCTGNGGDFIGA | SEQ. ID. NO.: 29 |
| Monoclonal Antibody | | |
| (Rabbit) | | |
| Light Chain CDR 3 | | |
| ST-3 Second | GFSLSSYYVR | SEQ. ID. NO.: 30 |
| Monoclonal Antibody | | |
| (Rabbit) | | |
| Heavy Chain CDR 1 | | |
| ST-3 Second | IISDSGSTYYASWAKG | SEQ. ID. NO.: 31 |
| Monoclonal Antibody | | |
| (Rabbit) | | |
| Heavy Chain CDR 2 | | |
| ST-3 Second | GSGYTIPTDL | SEQ. ID. NO.: 32 |
| Monoclonal Antibody | | |
| (Rabbit) | | |
| Heavy Chain CDR 3 | | |
| ST-3 First Monoclonal | DVVMTQTPLSLPVSLGDQASISCRSSQSLVHSNGNTYLH | SEQ. ID. NO.: 33 |
| Antibody | WYLQKPGQSPKLLIYKVSNRFSGVPDRFSGSGSGTDFTL | |
| (Human) | KISRVEAEDLGVYFCSQSTHVPYTFGGGTKLEIK | |
| Variable Light Chain | | |
| ST-3 First Monoclonal | QVQLKQSGPGLVQPSQSLSITCTVSGFSLTSYGVHWVR | SEQ. ID. NO.: 34 |
| Antibody | QSPGKGLEWLGVIWSGGSTDYNAAFISRLSISKDNSKSQ | |
| (Human) | VFFKMNSLQANDTAIYYCARNDYDRPIFAYWGQGTLV | |
| Variable Heavy Chain | TVSA | |
| ST-3 Second | DPVMTQTPASVSEPVGGTVTIKCQASQSIGSSLAWYQQ | SEQ. ID. NO.: 35 |
| Monoclonal Antibody | KPGQRPKLLIYQASKLASGVPSRFKGSRSGTEFTLTISDL | |
| (Rabbit) | ECADAATYYCQCTGNGGDFIGAFGGGTEVVVK | |
| Variable Light Chain | | |
| ST-3 Second | QSLEESGGGLVTPGTPLTLTCTASGFSLSSYYVRWVRQ | SEQ. ID. NO.: 36 |
| Monoclonal Antibody | APGKGLEYIGIISDSGSTYYASWAKGRFTISKTSTTVDLK | |
| (Rabbit) | FTSPTTEDTATYFCARGSGYTIPTDLWGPGTLVTVSS | |
| Variable Heavy Chain | | |
| ST-3 First Monoclonal | DVVMTQTPLSLPVSLGDQASISCRSSQSLVHSNGNTYLH | SEQ. ID. NO.: 37 |
| Antibody | WYLQKPGQSPKLLIYKVSNRFSGVPDRFSGSGSGTDFTL | |
| (Human) | KISRVEAEDLGVYFCSQSTHVPYTFGGGTKLEIKRTVAA | |
| Full Length Light Chain | PSVFIFPPSDEQLKSGTASVVCLLNNFYPREAKVQWKV | |
| | DNALQSGNSQESVTEQDSKDSTYSLSSTLTLSKADYEK | |
| | HKVYACEVTHQGLSSPVTKSFNRGEC | |
| ST-3 First Monoclonal | QVQLKQSGPGLVQPSQSLSITCTVSGFSLTSYGVHWVR | SEQ. ID. NO.: 38 |
| Antibody | QSPGKGLEWLGVIWSGGSTDYNAAFISRLSISKDNSKSQ | |
| (Human) | VFFKMNSLQANDTAIYYCARNDYDRPIFAYWGQGTLV | |
| Full Length Heavy | TVSAASTKGPSVFPLAPSSKSTSGGTAALGCLVKDYFPE | |
| Chain | PVTVSWNSGALTSGVHTFPAVLQSSGLYSLSSVVTVPSS | |
| | SLGTQTYICNVNHKPSNTKVDKKVEPKSCDKTHTCPPC | |
| | PAPELLGGPSVFLFPPKPKDTLMISRTPEVTCVVVDVSH | |
| | EDPEVKFNWYVDGVEVHNAKTKPREEQYNSTYRVVSV | |
| | LTVLHQDWLNGKEYKCKVSNKALPAPIEKTISKAKGQP | |
| | REPQVYTLPPSRDELTKNQVSLTCLVKGFYPSDIAVEWE | |
| | SNGQPENNYKTTPPVLDSDGSFFLYSKLTVDKSRWQQG | |
| | NVFSCSVMHEALHNHYTQKSLSL SPGK | |
| ST-3 Second | DPVMTQTPASVSEPVGGTVTIKCQASQSIGSSLAWYQQ | SEQ. ID. NO.: 39 |
| Monoclonal Antibody | KPGQRPKLLIYQASKLASGVPSRFKGSRSGTEFTLTISDL | |
| (Rabbit) | ECADAATYYCQCTGNGGDFIGAFGGGTEVVVKGDPVA | |
| Full Length Light Chain | PTVLIFPPAADQVATGTVTIVCVANKYFPDVTVTWEVD | |
| | GTTQTTGIENSKTPQNSADCTYNLSSTLTLTSTQYNSHK | |
| | EYTCKVTQGTTSVVQSFNRGDC | |
| ST-3 Second | QSLEESGGGLVTPGTPLTLTCTASGFSLSSYYVRWVRQ | SEQ. ID. NO.: 40 |
| Monoclonal Antibody | APGKGLEYIGIISDSGSTYYASWAKGRFTISKTSTTVDLK | |
| (Rabbit) | FTSPTTEDTATYFCARGSGYTIPTDLWGPGTLVTVSSGQ | |
| Full Length Heavy | PKAPSVFPLAPCCGDTPSSTVTLGCLVKGYLPEPVTVTW | |
| Chain | NSGTLTNGVRTFPSVRQSSGLYSLSSVVSVTSSSQPVTC | |
| | NVAHPATNTKVDKTVAPSTCSKPTCPPPELLGGPSVFIFP | |
| | PKPKDTLMISRTPEVTCVVVDVSQDDPEVQFTWYINNE | |
| | QVRTARPPLREQQFNSTIRVVSTLPIAHQDWLRGKEFKC | |
| | KVHNKALPAPIEKTISKARGQPLEPKVYTMGPPREELSS | |
| | RSVSLTCMINGFYPSDISVEWEKNGKAEDNYKTTPAVL | |
| | DSDGSYFLYSKLSVPTSEWQRGDVFTC | |
| | SVMHEALHNHYTQKSISRSPGK | |
| ST-4 First Monoclonal | KSSQSLLYSSNQRTYLA | SEQ. ID. NO.: 41 |
| Antibody (Mouse) | | |
| Light Chain CDR 1 | | |
| ST-4 First Monoclonal | WVSTREF | SEQ. ID. NO.: 42 |
| Antibody | | |
| (Mouse) | | |
| Light Chain CDR 2 | | |
| ST-4 First Monoclonal | HQYLSMFT | SEQ. ID. NO.: 43 |
| Antibody | | |
| (Mouse) | | |
| Light Chain CDR 3 | | |
| ST-4 First Monoclonal | GFTFSSFGMH | SEQ. ID. NO.: 44 |
| Antibody | | |
| (Mouse) | | |
| Heavy Chain CDR 1 | | |
| ST-4 First Monoclonal | YISSDNAIYYADTVEG | SEQ. ID. NO.: 45 |
| Antibody | | |
| (Mouse) | | |
| Heavy Chain CDR 2 | | |
| ST-4 First Monoclonal | SAWYFNV | SEQ. ID. NO.: 46 |
| Antibody | | |
| (Mouse) | | |
| Heavy Chain CDR 3 | | |
| ST-4 Second | KSSQSLLYSSNQRTYLA | SEQ. ID. NO.: 47 |
| Monoclonal Antibody | | |
| (Rabbit) | | |
| Light Chain CDR 1 | | |
| ST-4 Second | WVSTREF | SEQ. ID. NO.: 48 |
| Monoclonal Antibody | | |
| (Rabbit) | | |
| Light Chain CDR 2 | | |
| ST-4 Second | HQYLSMFT | SEQ. ID. NO.: 49 |
| Monoclonal Antibody | | |
| (Rabbit) | | |
| Light Chain CDR 3 | | |
| ST-4 Second | GFTFSSFGMH | SEQ. ID. NO.: 50 |
| Monoclonal Antibody | | |
| (Rabbit) | | |
| Heavy Chain CDR 1 | | |
| ST-4 Second | YISSDNAIYYADTVEG | SEQ. ID. NO.: 51 |
| Monoclonal Antibody | | |
| (Rabbit) | | |
| Heavy Chain CDR 2 | | |
| ST-4 Second | SAWYFNV | SEQ. ID. NO.: 52 |
| Monoclonal Antibody | | |
| (Rabbit) | | |
| Heavy Chain CDR 3 | | |
| ST-4 First Monoclonal | KIIMTQSPSSLAVSAGEEVTMTCKSSQSLLYSSNQRTYL | SEQ. ID. NO.: 53 |
| Antibody | AWYQQKPGQSPKLLIYWVSTREFGVPDRFTGSGSGTDF | |
| (Mouse) | TLTISSVQPEDLAVYYCHQYLSMFTFGSGTRLELK | |
| Variable Light Chain | | |
| ST-4 First Monoclonal | DVQLVESGGGLVQPGGSRKLSCAASGFTFSSFGMHWV | SEQ. ID. NO.: 54 |
| Antibody | RQAPEKGLEWVAYISSDNAIYYADTVEGRFTISRDNPK | |
| (Mouse) | NTLFLQMTSLRSEDTAIYYCARSAWYFNVWGAGTTVT | |
| Variable Heavy Chain | VSS | |
| ST-4 Second | KIIMTQSPSSLAVSAGEEVTMTCKSSQSLLYSSNQRTYL | SEQ. ID. NO.: 55 |
| Monoclonal Antibody | AWYQQKPGQSPKLLIYWVSTREFGVPDRFTGSGSGTDF | |
| (Rabbit) | TLTISSVQPEDLAVYYCHQYLSMFTFGSGTRLELK | |
| Variable Light Chain | | |
| ST-4 Second | DVQLVESGGGLVQPGGSRKLSCAASGFTFSSFGMHWV | SEQ. ID. NO.: 56 |
| Monoclonal Antibody | RQAPEKGLEWVAYISSDNAIYYADTVEGRFTISRDNPK | |
| (Rabbit) | NTLFLQMTSLRSEDTAIYYCARSAWYFNVWGAGTTVT | |
| Variable Heavy Chain | VSS | |
| ST-4 First Monoclonal | KIIMTQSPSSLAVSAGEEVTMTCKSSQSLLYSSNQRTYL | SEQ. ID. NO.: 57 |
| Antibody | AWYQQKPGQSPKLLIYWVSTREFGVPDRFTGSGSGTDF | |
| (Mouse) | TLTISSVQPEDLAVYYCHQYLSMFTFGSGTRLELKRAD | |
| Full Length Light Chain | AAPTVSIFPPSSEQLTSGGASVVCFLNNFYPKDINVKWKI | |
| | DGSERQNGVLNSWTDQDSKDSTYSMSSTLTLTKDEYER | |
| | HNSYTCEATHKTSTSPIVKSFNRNEC | |
| ST-4 First Monoclonal | DVQLVESGGGLVQPGGSRKLSCAASGFTFSSFGMHWV | SEQ. ID. NO.: 58 |
| Antibody | RQAPEKGLEWVAYISSDNAIYYADTVEGRFTIS | |
| (Mouse) | RDNPKNTLFLQMTSLRSEDTAIYYCARSAWYFNVWGA | |
| Full Length Heavy | GTTVTVSSAKTTPPSVYPLAPGSAAQTNSMVTLGCLVK | |
| Chain | GYFPEPVTVTWNSGSLSSGVHTFPAVLQSDLYTLSSSVT | |
| | VPSSTWPSETVTCNVAHPASSTKVDKKIVPRDCGCKPCI | |
| | CTVPEVSSVFIFPPKPKDVLTITLTPKVTCVVVDISKDDP | |
| | EVQFSWFVDDVEVHTAQTQPREEQFNSTFRSVSELPIM | |
| | HQDWLNGKEFKCRVNSAAFPAPIEKTISKTKGRPKAPQ | |
| | VYTIPPPKEQMAKDKVSLTCMITDFFPEDITVEWQWNG | |
| | QPAENYKNTQPIMDTDGSYFVYSKLNVQKSNWEAGNT | |
| | FTCSVLHEGLHNHHTEKSLSHSPGK | |
| ST-4 Second | KIIMTQSPSSLAVSAGEEVTMTCKSSQSLLYSSNQRTYL | SEQ. ID. NO.: 59 |
| Monoclonal Antibody | AWYQQKPGQSPKLLIYWVSTREFGVPDRFTGSGSGTDF | |
| (Rabbit) | TLTISSVQPEDLAVYYCHQYLSMFTFGSGTRLELKRDPV | |
| Full Length Light Chain | APTVLIFPPAADQVATGTVTIVCVANKYFPDVTVTWEV | |
| | DGTTQTTGIENSKTPQNSADCTYNLSSTLTLTSTQYNSH | |
| | KEYTCKVTQGTTSVVQSFNRGDC | |
| ST-4 Second | DVQLVESGGGLVQPGGSRKLSCAASGFTFSSFGMHWV | SEQ. ID. NO.: 60 |
| Monoclonal Antibody | RQAPEKGLEWVAYISSDNAIYYADTVEGRFTISRDNPK | |
| (Rabbit) | NTLFLQMTSLRSEDTAIYYCARSAWYFNVWGAGTTVT | |
| Full Length Heavy | VSSGQPKAPSVFPLAPCCGDTPSSTVTLGCLVKGYLPEP | |
| Chain | VTVTWNSGTLTNGVRTFPSVRQSSGLYSLSSVVSVTSSS | |
| | QPVTCNVAHPATNTKVDKTVAPSTCSKPTCPPPELLGGP | |
| | SVFIFPPKPKDTLMISRTPEVTCVVVDVSEDDPEVQFTW | |
| | YINNEQVRTARPPLREQQFNSTIRVVSTLPIAHEDWLRG | |
| | KEFKCKVHNKALPAPIEKTISKARGQPLEPKVYTMGPPR | |
| | EELSSRSVSLTCMINGFYPSDISVEWEKNGKAEDNYKTT | |
| | PAVLDSDGSYFLYSKLSVPTSEWQRGDVFTCSVMHEAL | |
| | HNHYTQKSISRSPGK | |
| ST-5 First Monoclonal | QASQNTDIRLA | SEQ. ID. NO.: 61 |
| Antibody (Mouse or | | |
| Human) | | |
| Light Chain CDR 1 | | |
| ST-5 First Monoclonal | SASTLAS | SEQ. ID. NO.: 62 |
| Antibody | | |
| (Human) | | |
| Light Chain CDR 2 | | |
| ST-5 First Monoclonal | LGNYDCSYADCYA | SEQ. ID. NO.: 63 |
| Antibody | | |
| (Human) | | |
| Light Chain CDR 3 | | |
| ST-5 First Monoclonal | GIDLNNYEMG | SEQ. ID. NO.: 64 |
| Antibody | | |
| (Human) | | |
| Heavy Chain CDR 1 | | |
| ST-5 First Monoclonal | YIRTGGSAYYASWAKG | SEQ. ID. NO.: 65 |
| Antibody | | |
| (Human) | | |
| Heavy Chain CDR 2 | | |
| ST-5 First Monoclonal | PYAFVSLINDL | SEQ. ID. NO.: 66 |
| Antibody | | |
| (Human) | | |
| Heavy Chain CDR 3 | | |
| ST-5 Second | QASQNTDIRLA | SEQ. ID. NO.: 67 |
| Monoclonal Antibody | | |
| (Rabbit) | | |
| Light Chain CDR 1 | | |
| ST-5 Second | SASTLAS | SEQ. ID. NO.: 68 |
| Monoclonal Antibody | | |
| (Rabbit) | | |
| Light Chain CDR 2 | | |
| ST-5 Second | LGNYDCSYADCYA | SEQ. ID. NO.: 69 |
| Monoclonal Antibody | | |
| (Rabbit) | | |
| Light Chain CDR 3 | | |
| ST-5 Second | GIDLNNYEMG | SEQ. ID. NO.: 70 |
| Monoclonal Antibody | | |
| (Rabbit) | | |
| Heavy Chain CDR 1 | | |
| ST-5 Second | YIRTGGSAYYASWAKG | SEQ. ID. NO.: 71 |
| Monoclonal Antibody | | |
| (Rabbit) | | |
| Heavy Chain CDR 2 | | |
| ST-5 Second | PYAFVSLINDL | SEQ. ID. NO.: 72 |
| Monoclonal Antibody | | |
| (Rabbit) | | |
| Heavy Chain CDR 3 | | |
| ST-5 First Monoclonal | AQVLTQTPSSVSAAVGGTVTINCQASQNTDIRLAWYQQ | SEQ. ID. NO.: 73 |
| Antibody | KPGQPPKRLIYSASTLASGVPSRFKGSGSGTQFTLTISDL | |
| (Human) | ECDDAATYYCLGNYDCSYADCYAFGGGTEVVVR | |
| Variable Light Chain | | |
| ST-5 First Monoclonal | QSLEESGGRLVTPGTPLTLTCTVSGIDLNNYEMGWVRQ | SEQ. ID. NO.: 74 |
| Antibody | APGKGLEWIGYIRTGGSAYYASWAKGRFTISKTSTTVD | |
| (Human) | LKMTSLATEDTATYFCARPYAFVSLINDLWGPGTLVTV | |
| Variable Heavy Chain | SS | |
| ST-5 Second | AQVLTQTPSSVSAAVGGTVTINCQASQNTDIRLAWYQQ | SEQ. ID. NO.: 75 |
| Monoclonal Antibody | KPGQPPKRLIYSASTLASGVPSRFKGSGSGTQFTLTISDL | |
| (Rabbit) | ECDDAATYYCLGNYDCSYADCYAFGGGTEVVVR | |
| Variable Light Chain | | |
| ST-5 Second | QSLEESGGRLVTPGTPLTLTCTVSGIDLNNYEMGWVRQ | SEQ. ID. NO.: 76 |
| Monoclonal Antibody | APGKGLEWIGYIRTGGSAYYASWAKGRFTISKTSTTVD | |
| (Rabbit) | LKMTSLATEDTATYFCARPYAFVSLINDLWGPGTLVTV | |
| Variable Heavy Chain | SS | |
| ST-5 First Monoclonal | AQVLTQTPSSVSAAVGGTVTINCQASQNTDIRLAWYQQ | SEQ. ID. NO.: 77 |
| Antibody | KPGQPPKRLIYSASTLASGVPSRFKGSGSGTQFTLTISDL | |
| (Human) | ECDDAATYYCLGNYDCSYADCYAFGGGTEVVVRRTV | |
| Full Length Light Chain | AAPSVFIFPPSDEQLKSGTASVVCLLNNFYPREAKVQW | |
| | KVDNALQSGNSQESVTEQDSKDSTYSLSSTLTLSKADY | |
| | EKHKVYACEVTHQGLSSPVTKSFNRGEC | |
| ST-5 First Monoclonal | QSLEESGGRLVTPGTPLTLTCTVSGIDLNNYEMGWVRQ | SEQ. ID. NO.: 78 |
| Antibody | APGKGLEWIGYIRTGGSAYYASWAKGRFTISKTSTTVD | |
| (Human) | LKMTSLATEDTATYFCARPYAFVSLINDLWGPGTLVTV | |
| Full Length Heavy | SSASTKGPSVFPLAPSSKSTSGGTAALGCLVKDYFPEPV | |
| Chain | TVSWNSGALTSGVHTFPAVLQSSGLYSLSSVVTVPSSSL | |
| | GTQTYICNVNHKPSNTKVDKKVEPKSCDKTHTCPPCPA | |
| | PELLGGPSVFLFPPKPKDTLMISRTPEVTCVVVDVSHED | |
| | PEVKFNWYVDGVEVHNAKTKPREEQYNSTYRVVSVLT | |
| | VLHQDWLNGKEYKCKVSNKALPAPIEKTISKAKGQPRE | |
| | PQVYTLPPSRDELTKNQVSLTCLVKGFYPSDIAVEWESN | |
| | GQPENNYKTTPPVLDSDGSFFLYSKLTVDKSRWQQGNV | |
| | FSCSVMHEALHNHYTQKSLSLSPGK | |
| ST-5 Second | AQVLTQTPSSVSAAVGGTVTINCQASQNTDIRLAWYQQ | SEQ. ID. NO.: 79 |
| Monoclonal Antibody | KPGQPPKRLIYSASTLASGVPSRFKGSGSGTQFTLTISDL | |
| (Rabbit) | ECDDAATYYCLGNYDCSYADCYAFGGGTEVVVRGDP | |
| Full Length Light Chain | VAPTVLIFPPAADQVATGTVTIVCVANKYFPDVTVTWE | |
| | VDGTTQTTGIENSKTPQNSADCTYNLSSTLTLTSTQYNS | |
| | HKEYTCKVTQGTTSVVQSFNRGDC | |
| ST-5 Second | QSLEESGGRLVTPGTPLTLTCTVSGIDLNNYEMGWVRQ | SEQ. ID. NO.: 80 |
| Monoclonal Antibody | APGKGLEWIGYIRTGGSAYYASWAKGRFTISKTSTTVD | |
| (Rabbit) | LKMTSLATEDTATYFCARPYAFVSLINDLWGPGTLVTV | |
| Full Length Heavy | SSGQPKAPSVFPLAPCCGDTPSSTVTLGCLVKGYLPEPV | |
| Chain | TVTWNSGTLTNGVRTFPSVRQSSGLYSLSSVVSVTSSSQ | |
| | PVTCNVAHPATNTKVDKTVAPSTCSKPTCPPPELLGGPS | |
| | VFIFPPKPKDTLMISRTPEVTCVVVDVSQDDPEVQFTWY | |
| | INNEQVRTARPPLREQQFNSTIRVVSTLPIAHQDWLRGK | |
| | EFKCKVHNKALPAPIEKTISKARGQPLEPKVYTMGPPRE | |
| | ELSSRSVSLTCMINGFYPSDISVEWEKNGKAEDNYKTTP | |
| | AVLDSDGSYFLYSKLSVPTSEWQRGDVFT | |
| | CSVMHEALHNHYTQKSISRSPGK | |
| ST-6A First Monoclonal | QASQSVWKNNYLS | SEQ. ID. NO.: 81 |
| Antibody (Mouse or | | |
| Human) | | |
| Light Chain CDR 1 | | |
| ST-6A First Monoclonal | TASSLAS | SEQ. ID. NO.: 82 |
| Antibody | | |
| (Human) | | |
| Light Chain CDR 2 | | |
| ST-6A First Monoclonal | AGDVGGGIRT | SEQ. ID. NO.: 83 |
| Antibody | | |
| (Human) | | |
| Light Chain CDR 3 | | |
| ST-6A First Monoclonal | GFSLSSYTTS | SEQ. ID. NO.: 84 |
| Antibody | | |
| (Human) | | |
| Heavy Chain CDR 1 | | |
| ST-6A First Monoclonal | VIDVGSDDTYYATWAKG | SEQ. ID. NO.: 85 |
| Antibody | | |
| (Human) | | |
| Heavy Chain CDR 2 | | |
| ST-6A First Monoclonal | HGATGGTVFDL | SEQ. ID. NO.: 86 |
| Antibody | | |
| (Human) | | |
| Heavy Chain CDR 3 | | |
| ST-6A Second | QASQSVWKNNYLS | SEQ. ID. NO.: 87 |
| Monoclonal Antibody | | |
| (Rabbit) | | |
| Light Chain CDR 1 | | |
| ST-6A Second | TASSLAS | SEQ. ID. NO.: 88 |
| Monoclonal Antibody | | |
| (Rabbit) | | |
| Light Chain CDR 2 | | |
| ST-6A Second | AGDVGGGIRT | SEQ. ID. NO.: 89 |
| Monoclonal Antibody | | |
| (Rabbit) | | |
| Light Chain CDR 3 | | |
| ST-6A Second | GFSLSSYTTS | SEQ. ID. NO.: 90 |
| Monoclonal Antibody | | |
| (Rabbit) | | |
| Heavy Chain CDR 1 | | |
| ST-6A Second | VIDVGSDDTYYATWAK | SEQ. ID. NO.: 91 |
| Monoclonal Antibody | | |
| (Rabbit) | | |
| Heavy Chain CDR 2 | | |
| ST-6A Second | HGATGGTVFDL | SEQ. ID. NO.: 92 |
| Monoclonal Antibody | | |
| (Rabbit) | | |
| Heavy Chain CDR 3 | | |
| ST-6A First Monoclonal | AQVLTQTPSPVSAAVGGTVTINCQASQSVWKNNYLSW | SEQ. ID. NO.: 93 |
| Antibody | FQQKPGQPPKLLIYTASSLASGVPSRFKGSGSGTQFTLTI | |
| (Human) | SDLECDDAATYYCAGDVGGGIRTFGGGTEVVVK | |
| Variable Light Chain | | |
| ST-6A First Monoclonal | QSLEESGGRLVTPGTPLTLTCTASGFSLSSYTTSWVRQA | SEQ. ID. NO.: 94 |
| Antibody | PGKGLEWVGVIDVGSDDTYYATWAKGRFTISRTSTTVD | |
| (Human) | LKMTSLTAADTATYFCTRHGATGGTVFDLWGPGTLVT | |
| Variable Heavy Chain | VSS | |
| ST-6A Second | AQVLTQTPSPVSAAVGGTVTINCQASQSVWKNNYLSW | SEQ. ID. NO.: 95 |
| Monoclonal Antibody | FQQKPGQPPKLLIYTASSLASGVPSRFKGSGSGQFTLTIS | |
| (Rabbit) | DLECDDAATYYCAGDVGGGIRTFGGGTEVVVK | |
| Variable Light Chain | | |
| ST-6A Second | QSLEESGGRLVTPGTPLTLTCTASGFSLSSYTTSWVRQA | SEQ. ID. NO.: 96 |
| Monoclonal Antibody | PGKGLEWVGVIDVGSDDTYYATWAKGRFTISRTSTTVD | |
| (Rabbit) | LKMTSLTAADTATYFCTRHGATGGTVFDLWGPGTLVT | |
| Variable Heavy Chain | VSS | |
| ST-6A First Monoclonal | AQVLTQTPSPVSAAVGGTVTINCQASQSVWKNNYLSW | SEQ. ID. NO.: 97 |
| Antibody | FQQKPGQPPKLLIYTASSLASGVPSRFKGSGSGTQFTLTI | |
| (Human) | SDLECDDAATYYCAGDVGGGIRTFGGGTEVVVKRTVA | |
| Full Length Light Chain | APSVFIFPPSDEQLKSGTASVVCLLNNFYPREAKVQWK | |
| | VDNALQSGNSQESVTEQDSKDSTYSLSSTLTLSKADYE | |
| | KHKVYACEVTHQGLSSPVTKSFNRGEC | |
| ST-6A First Monoclonal | QSLEESGGRLVTPGTPLTLTCTASGFSLSSYTTSWVRQA | SEQ. ID. NO.: 98 |
| Antibody | PGKGLEWVGVIDVGSDDTYYATWAKGRFTISRTSTTVD | |
| (Human) | LKMTSLTAADTATYFCTRHGATGGTVFDLWGPGTLVT | |
| Full Length Heavy | VSSASTKGPSVFPLAPSSKSTSGGTAALGCLVKDYFPEP | |
| Chain | VTVSWNSGALTSGVHTFPAVLQSSGLYSLSSVVTVPSSS | |
| | LGTQTYICNVNHKPSNTKVDKKVEPKSCDKTHTCPPCP | |
| | APELLGGPSVFLFPPKPKDTLMISRTPEVTCVVVDVSHE | |
| | DPEVKFNWYVDGVEVHNAKTKPREEQYNSTYRVVSVL | |
| | TVLHQDWLNGKEYKCKVSNKALPAPIEKTISKAKGQPR | |
| | EPQVYTLPPSRDELTKNQVSLTCLVKGFYPSDIAVEWES | |
| | NGQPENNYKTTPPVLDSDGSFFLYSKLTVDKSRWQQGN | |
| | VFSCSVMHEALHNHYTQKSLSLS PGK | |
| ST-6A Second | AQVLTQTPSPVSAAVGGTVTINCQASQSVWKNNYLSW | SEQ. ID. NO.: 99 |
| Monoclonal Antibody | FQQKPGQPPKLLIYTASSLASGVPSRFKGSGSGTQFTLTI | |
| (Rabbit) | SDLECDDAATYYCAGDVGGGIRTFGGGTEVVVKGDPV | |
| Full Length Light Chain | APTVLIFPPAADQVATGTVTIVCVANKYFPDVTVTWEV | |
| | DGTTQTTGIENSKTPQNSADCTYNLSSTLTLTSTQYNSH | |
| | KEYTCKVTQGTTSVVQSFNRGDC | |
| ST-6A Second | QSLEESGGRLVTPGTPLTLTCTASGFSLSSYTTSWVRQA | SEQ. ID. NO.: |
| Monoclonal Antibody | PGKGLEWVGVIDVGSDDTYYATWAKGRFTISRTSTTVD | 100 |
| (Rabbit) | LKMTSLTAADTATYFCTRHGATGGTVFDLWGPGTLVT | |
| Full Length Heavy | VSSGQPKAPSVFPLAPCCGDTPSSTVTLGCLVKGYLPEP | |
| Chain | VTVTWNSGTLTNGVRTFPSVRQSSGLYSLSSVVSVTSSS | |
| | QPVTCNVAHPATNTKVDKTVAPSTCSKPTCPPPELLGGP | |
| | SVFIFPPKPKDTLMISRTPEVTCVVVDVSQDDPEVQFTW | |
| | YINNEQVRTARPPLREQQFNSTIRVVSTLPIAHQDWLRG | |
| | KEFKCKVHNKALPAPIEKTISKARGQPLEPKVYTMGPPR | |
| | EELSSRSVSLTCMINGFYPSDISVEWEKNGKAEDNYKTT | |
| | PAVLDSDGSYFLYSKLSVPTSEWQRGDVFTCSVMHEAL | |
| | HNHYTQKSISRSPGK | |
| ST-6B First Monoclonal | KSSQSVLYSSNQKNYLA | SEQ. ID. NO.: |
| Antibody (Mouse) | | 101 |
| Light Chain CDR 1 | | |
| ST-6B First Monoclonal | WASTRES | SEQ. ID. NO.: |
| Antibody | | 102 |
| (Mouse) | | |
| Light Chain CDR 2 | | |
| ST-6B First Monoclonal | HQHLSSWT | SEQ. ID. NO.: |
| Antibody | | 103 |
| (Mouse) | | |
| Light Chain CDR 3 | | |
| ST-6B First Monoclonal | GFTFRNYGMS | SEQ. ID. NO.: |
| Antibody | | 104 |
| (Mouse) | | |
| Heavy Chain CDR 1 | | |
| ST-6B First Monoclonal | VINSNGGKTYYPDSVKG | SEQ. ID. NO.: |
| Antibody | | 105 |
| (Mouse) | | |
| Heavy Chain CDR 2 | | |
| ST-6B First Monoclonal | DGKGWFAY | SEQ. ID. NO.: |
| Antibody | | 106 |
| (Mouse) | | |
| Heavy Chain CDR 3 | | |
| ST-6B Second | KSSQSVLYSSNQKNYLA | SEQ. ID. NO.: |
| Monoclonal Antibody | | 107 |
| (Rabbit) | | |
| Light Chain CDR 1 | | |
| ST-6B Second | WASTRES | SEQ. ID. NO.: |
| Monoclonal Antibody | | 108 |
| (Rabbit) | | |
| Light Chain CDR 2 | | |
| ST-6B Second | HQHLSSWT | SEQ. ID. NO.: |
| Monoclonal Antibody | | 109 |
| (Rabbit) | | |
| Light Chain CDR 3 | | |
| ST-6B Second | GFTFRNYGMS | SEQ. ID. NO.: |
| Monoclonal Antibody | | 110 |
| (Rabbit) | | |
| Heavy Chain CDR 1 | | |
| ST-6B Second | VINSNGGKTYYPDSVKG | SEQ. ID. NO.: |
| Monoclonal Antibody | | 111 |
| (Rabbit) | | |
| Heavy Chain CDR 2 | | |
| ST-6B Second | DGKGWFAY | SEQ. ID. NO.: |
| Monoclonal Antibody | | 112 |
| (Rabbit) | | |
| Heavy Chain CDR 3 | | |
| ST-6B First Monoclonal | NIMMTQSPSSLAVSAGEKVTMNCKSSQSVLYSSNQKNY | SEQ. ID. NO.: |
| Antibody | LAWYQQKPGQSPKLLIYWASTRESGVPDRFTGSGSGTD | 113 |
| (Mouse) | FALTISSVQLEDLAVYYCHQHLSSWTFGGGTKVEIK | |
| Variable Light Chain | | |
| ST-6B First Monoclonal | EVQLVESGGGLVQPGGSLKLSCAASGFTFRNYGMSWV | SEQ. ID. NO.: |
| Antibody | RQTPDKRLELVAVINSNGGKTYYPDSVKGRFTISRDNA | 114 |
| (Mouse) | KNTLYLQMSSLKSEDTAMYYCARDGKGWFAYWGQGT | |
| Variable Heavy Chain | LVTVSA | |
| ST-6B Second | NIMMTQSPSSLAVSAGEKVTMNCKSSQSVLYSSNQKNY | SEQ. ID. NO.: |
| Monoclonal Antibody | LAWYQQKPGQSPKLLIYWASTRESGVPDRFTGSGSGTD | 115 |
| (Rabbit) | FALTISSVQLEDLAVYYCHQHLSSWTFGGGTKVEIK | |
| Variable Light Chain | | |
| ST-6B Second | EVQLVESGGGLVQPGGSLKLSCAASGFTFRNYGMSWV | SEQ. ID. NO.: |
| Monoclonal Antibody | RQTPDKRLELVAVINSNGGKTYYPDSVKGRFTISRDNA | 116 |
| (Rabbit) | KNTLYLQMSSLKSEDTAMYYCARDGKGWFAYWGQGT | |
| Variable Heavy Chain | LVTVSA | |
| ST-6B First Monoclonal | NIMMTQSPSSLAVSAGEKVTMNCKSSQSVLYSSNQKNY | SEQ. ID. NO.: |
| Antibody | LAWYQQKPGQSPKLLIYWASTRESGVPDRFTGSGSGTD | 117 |
| (Mouse) | FALTISSVQLEDLAVYYCHQHLSSWTFGGGTKVEIKRA | |
| Full Length Light Chain | DAAPTVSIFPPSSEQLTSGGASVVCFLNNFYPKDINVKW | |
| | KIDGSERQNGVLNSWTDQDSKDSTYSMSSTLTLTKDEY | |
| | ERHNSYTCEATHKTSTSPIVKSFNRNEC | |
| ST-6B First Monoclonal | EVQLVESGGGLVQPGGSLKLSCAASGFTFRNYGMSWV | SEQ. ID. NO.: |
| Antibody | RQTPDKRLELVAVINSNGGKTYYPDSVKGRFTISRDNA | 118 |
| (Mouse) | KNTLYLQMSSLKSEDTAMYYCARDGKGWFAYWGQGT | |
| Full Length Heavy | LVTVSAAKTTPPSVYPLAPGSAAQTNSMVTLGCLVKGY | |
| Chain | FPEPVTVTWNSGSLSSGVHTFPAVLQSDLYTLSSSVTVP | |
| | SSTWPSETVTCNVAHPASSTKVDKKIVPRDCGCKPCICT | |
| | VPEVSSVFIFPPKPKDVLTITLTPKVTCVVVDISKDDPEV | |
| | QFSWFVDDVEVHTAQTQPREEQFNSTFRSVSELPIMHQ | |
| | DWLNGKEFKCRVNSAAFPAPIEKTISKTKGRPKAPQVY | |
| | TIPPPKEQMAKDKVSLTCMITDFFPEDITVEWQWNGQP | |
| | AENYKNTQPIMDTDGSYFVYSKLNVQKSNWEAGNTFT | |
| | CSVLHEGLHNHHTEKSLSHSPGK | |
| ST-6B Second | NIMMTQSPSSLAVSAGEKVTMNCKSSQSVLYSSNQKNY | SEQ. ID. NO.: |
| Monoclonal Antibody | LAWYQQKPGQSPKLLIYWASTRESGVPDRFTGSGSGTD | 119 |
| (Rabbit) | FALTISSVQLEDLAVYYCHQHLSSWTFGGGTKVEIKRDP | |
| Full Length Light Chain | VAPTVLIFPPAADQVATGTVTIVCVANKYFPDVTVTWE | |
| | VDGTTQTTGIENSKTPQNSADCTYNLSSTLTLTSTQYNS | |
| | HKEYTCKVTQGTTSVVQSFNRGDC | |
| ST-6B Second | EVQLVESGGGLVQPGGSLKLSCAASGFTFRNYGMSWV | SEQ. ID. NO.: |
| Monoclonal Antibody | RQTPDKRLELVAVINSNGGKTYYPDSVKGRFTISRDNA | 120 |
| (Rabbit) | KNTLYLQMSSLKSEDTAMYYCARDGKGWFAYWGQGT | |
| Full Length Heavy | LVTVSAGQPKAPSVFPLAPCCGDTPSSTVTLGCLVKGYL | |
| Chain | PEPVTVTWNSGTLTNGVRTFPSVRQSSGLYSLSSVVSVT | |
| | SSSQPVTCNVAHPATNTKVDKTVAPSTCSKPTCPPPELL | |
| | GGPSVFIFPPKPKDTLMISRTPEVTCVVVDVSEDDPEVQ | |
| | FTWYINNEQVRTARPPLREQQFNSTIRVVSTLPIAHEDW | |
| | LRGKEFKCKVHNKALPAPIEKTISKARGQPLEPKVYTM | |
| | GPPREELSSRSVSLTCMINGFYPSDISVEWEKNGKAEDN | |
| | YKTTPAVLDSDGSYFLYSKLSVPTSEWQRGDVFTCSVM | |
| | HEALHNHYTQKSISRSPGK | |
| ST-7F First Monoclonal | TGNSNNVGNQGAA | SEQ. ID. NO.: |
| Antibody (Human) | | 121 |
| Light Chain CDR 1 | | |
| ST-7F First Monoclonal | RNNNRPS | SEQ. ID. NO.: |
| Antibody | | 122 |
| (Human) | | |
| Light Chain CDR 2 | | |
| ST-7F First Monoclonal | SAWDSSLNAWV | SEQ. ID. NO.: |
| Antibody | | 123 |
| (Human) | | |
| Light Chain CDR 3 | | |
| ST-7F First Monoclonal | GFTFSNYVMH | SEQ. ID. NO.: |
| Antibody | | 124 |
| (Human) | | |
| Heavy Chain CDR 1 | | |
| ST-7F First Monoclonal | LIWSDGSTIFHADSVKG | SEQ. ID. NO.: |
| Antibody | | 125 |
| (Human) | | |
| Heavy Chain CDR 2 | | |
| ST-7F First Monoclonal | EPRAIADNYYGMDV | SEQ. ID. NO.: |
| Antibody | | 126 |
| (Human) | | |
| Heavy Chain CDR 3 | | |
| ST-7F Second | TGNSNNVGNQGAA | SEQ. ID. NO.: |
| Monoclonal Antibody | | 127 |
| (Rabbit) | | |
| Light Chain CDR 1 | | |
| ST-7F Second | RNNNRPS | SEQ. ID. NO.: |
| Monoclonal Antibody | | 128 |
| (Rabbit) | | |
| Light Chain CDR 2 | | |
| ST-7F Second | SAWDSSLNAWV | SEQ. ID. NO.: |
| Monoclonal Antibody | | 129 |
| (Rabbit) | | |
| Light Chain CDR 3 | | |
| ST-7F Second | GFTFSNYVMH | SEQ. ID. NO.: |
| Monoclonal Antibody | | 130 |
| (Rabbit) | | |
| Heavy Chain CDR 1 | | |
| ST-7F Second | LIWSDGSTIFHADSVKG | SEQ. ID. NO.: |
| Monoclonal Antibody | | 131 |
| (Rabbit) | | |
| Heavy Chain CDR 2 | | |
| ST-7F Second | EPRAIADNYYGMDV | SEQ. ID. NO.: |
| Monoclonal Antibody | | 132 |
| (Rabbit) | | |
| Heavy Chain CDR 3 | | |
| ST-7F First Monoclonal | QAGLTQPPSVSKGLRQTATLTCTGNSNNVGNQGAAWL | SEQ. ID. NO.: |
| Antibody | QQHQGHPPKLLSYRNNNRPSGISERLSASRSGNTASLTIT | 133 |
| (Human) | GLQPEDEADYYCSAWDSSLNAWVFGGGTKLAVL | |
| Variable Light Chain | | |
| ST-7F First Monoclonal | QVQLVESGGDVVQPGRSLRLSCAASGFTFSNYVMHWV | SEQ. ID. NO.: |
| Antibody | RQAPGEGLEWVSLIWSDGSTIFHADSVKGRFTISRDNSK | 134 |
| (Human) | NTLYLQMDSLRAEDTAVYYCAREPRAIADNYYGMDV | |
| Variable Heavy Chain | WGQGTSVTVSS | |
| ST-7F Second | QAGLTQPPSVSKGLRQTATLTCTGNSNNVGNQGAAWL | SEQ. ID. NO.: |
| Monoclonal Antibody | QQHQGHPPKLLSYRNNNRPSGISERLSASRSGNTASLTIT | 135 |
| (Rabbit) | GLQPEDEADYYCSAWDSSLNAWVFGGGTKLAVL | |
| Variable Light Chain | | |
| ST-7F Second | QVQLVESGGDVVQPGRSLRLSCAASGFTFSNYVMHWV | SEQ. ID. NO.: |
| Monoclonal Antibody | RQAPGEGLEWVSLIWSDGSTIFHADSVKGRFTISRDNSK | 136 |
| (Rabbit) | NTLYLQMDSLRAEDTAVYYCAREPRAIADNYYGMDV | |
| Variable Heavy Chain | WGQGTSVTVSS | |
| ST-7F First Monoclonal | QAGLTQPPSVSKGLRQTATLTCTGNSNNVGNQGAAWL | SEQ. ID. NO.: |
| Antibody | QQHQGHPPKLLSYRNNNRPSGISERLSASRSGNTASLTIT | 137 |
| (Human) | GLQPEDEADYYCSAWDSSLNAWVFGGGTKLAVLGQPK | |
| Full Length Light Chain | AAPSVTLFPPSSEELQANKATLVCLISDFYPGAVTVAWK | |
| | ADSSPVKAGVETTTPSKQSNNKYAASSYLSLTPEQWKS | |
| | HRSYSCQVTHEGSTVEKTVAPTECS | |
| ST-7F First Monoclonal | QVQLVESGGDVVQPGRSLRLSCAASGFTFSNYVMHWV | SEQ. ID. NO.: |
| Antibody | RQAPGEGLEWVSLIWSDGSTIFHADSVKGRFTISRDNSK | 138 |
| (Human) | NTLYLQMDSLRAEDTAVYYCAREPRAIADNYYGMDV | |
| Full Length Heavy | WGQGTSVTVSSASTKGPSVFPLAPSSKSTSGGTAALGCL | |
| Chain | VKDYFPEPVTVSWNSGALTSGVHTFPAVLQSSGLYSLSS | |
| | VVTVPSSSLGTQTYICNVNHKPSNTKVDKKVEPKSCDK | |
| | THTCPPCPAPELLGGPSVFLFPPKPKDTLMISRTPEVTCV | |
| | VVDVSHEDPEVKFNWYVDGVEVHNAKTKPREEQYNST | |
| | YRVVSVLTVLHQDWLNGKEYKCKVSNKALPAPIEKTIS | |
| | KAKGQPREPQVYTLPPSRDELTKNQVSLTCLVKGFYPS | |
| | DIAVEWESNGQPENNYKTTPPVLDSDGSFFLYSKLTVD | |
| | KSRWQQGNVFSCSVMHEALHNHYTQKSLSLSPGK | |
| ST-7F Second | QAGLTQPPSVSKGLRQTATLTCTGNSNNVGNQGAAWL | SEQ. ID. NO.: |
| Monoclonal Antibody | QQHQGHPPKLLSYRNNNRPSGISERLSASRSGNTASLTIT | 139 |
| (Rabbit) | GLQPEDEADYYCSAWDSSLNAWVFGGGTKLAVLGQPA | |
| Full Length Light Chain | VTPSVILFPPSSEELKDNKATLVCLISDFYPRTVKVNWK | |
| | ADGNSVTQGVDTTQPSKQSNNKYAASSFLHLTANQWK | |
| | SYQSVTCQVTHEGHTVEKSLAPAECS | |
| ST-7F Second | QVQLVESGGDVVQPGRSLRLSCAASGFTFSNYVMHWV | SEQ. ID. NO.: |
| Monoclonal Antibody | RQAPGEGLEWVSLIWSDGSTIFHADSVKGRFTISRDNSK | 140 |
| (Rabbit) | NTLYLQMDSLRAEDTAVYYCAREPRAIADNYYGMDV | |
| Full Length Heavy | WGQGTSVTVSSGQPKAPSVFPLAPCCGDTPSSTVTLGCL | |
| Chain | VKGYLPEPVTVTWNSGTLTNGVRTFPSVRQSSGLYSLSS | |
| | VVSVTSSSQPVTCNVAHPATNTKVDKTVAPSTCSKPTC | |
| | PPPELLGGPSVFIFPPKPKDTLMISRTPEVTCVVVDVSQD | |
| | DPEVQFTWYINNEQVRTARPPLREQQFNSTIRVVSTLPI | |
| | AHQDWLRGKEFKCKVHNKALPAPIEKTISKARGQPLEP | |
| | KVYTMGPPREELSSRSVSLTCMINGFYPSDISVEWEKNG | |
| | KAEDNYKTTPAVLDSDGSYFLYSKLSVPTSEWQRGDVF | |
| | TCSVMHEALHNHYTQKSISRSPGK | |
| ST-9V First Monoclonal | QASKTVYDDNALA | SEQ. ID. NO.: |
| Antibody (Mouse or | | 141 |
| Human) | | |
| Light Chain CDR 1 | | |
| ST-9V First Monoclonal | KASTLAS | SEQ. ID. NO.: |
| Antibody | | 142 |
| (Mouse or Human) | | |
| Light Chain CDR 2 | | |
| ST-9V First Monoclonal | AGGYIYDSGDHA | SEQ. ID. NO.: |
| Antibody | | 143 |
| (Mouse) | | |
| Light Chain CDR 3 | | |
| ST-9V First Monoclonal | GIDLSRGQVG | SEQ. ID. NO.: |
| Antibody | | 144 |
| (Mouse) | | |
| Heavy Chain CDR 1 | | |
| ST-9V First Monoclonal | FKGYGGNAFYTNWAKG | SEQ. ID. NO.: |
| Antibody | | 145 |
| (Mouse) | | |
| Heavy Chain CDR 2 | | |
| ST-9V First Monoclonal | VAGDINHLDL | SEQ. ID. NO.: |
| Antibody | | 146 |
| (Mouse) | | |
| Heavy Chain CDR 3 | | |
| ST-9V Second | QASKTVYDDNALA | SEQ. ID. NO.: |
| Monoclonal Antibody | | 147 |
| (Rabbit) | | |
| Light Chain CDR 1 | | |
| ST-9V Second | KASTLAS | SEQ. ID. NO.: |
| Monoclonal Antibody | | 148 |
| (Rabbit) | | |
| Light Chain CDR 2 | | |
| ST-9V Second | AGGYIYDSGDHA | SEQ. ID. NO.: |
| Monoclonal Antibody | | 149 |
| (Rabbit) | | |
| Light Chain CDR 3 | | |
| ST-9V Second | GIDLSRGQVG | SEQ. ID. NO.: |
| Monoclonal Antibody | | 150 |
| (Rabbit) | | |
| Heavy Chain CDR 1 | | |
| ST-9V Second | FKGYGGNAFYTNWAKG | SEQ. ID. NO.: |
| Monoclonal Antibody | | 151 |
| (Rabbit) | | |
| Heavy Chain CDR 2 | | |
| ST-9V Second | VAGDINHLDL | SEQ. ID. NO.: |
| Monoclonal Antibody | | 152 |
| (Rabbit) | | |
| Heavy Chain CDR 3 | | |
| ST-9V First Monoclonal | AAVLTQTPSPVSAAVGGTVSINCQASKTVYDDNALAW | SEQ. ID. NO.: |
| Antibody | YQQKPGQPPKLLIYKASTLASGVPSRFSGSGSGTQFTLTI | 153 |
| (Mouse) | SDVQCDDAATYYCAGGYIYDSGDHAFGGGTEVVVK | |
| Variable Light Chain | | |
| ST-9V First Monoclonal | QSLEESGGRLVTPGTPLTLTCTVSGIDLSRGQVGWVRQ | SEQ. ID. NO.: |
| Antibody | APGEGLEYIGFKGYGGNAFYTNWAKGRFTISKTSSTTV | 154 |
| (Mouse) | DLKITTPTTEDTATYFCARVAGDINHLDLWGQGTLVTV | |
| Variable Heavy Chain | SS | |
| ST-9V Second | AAVLTQTPSPVSAAVGGTVSINCQASKTVYDDNALAW | SEQ. ID. NO.: |
| Monoclonal Antibody | YQQKPGQPPKLLIYKASTLASGVPSRFSGSGSGTQFTLTI | 155 |
| (Rabbit) | SDVQCDDAATYYCAGGYIYDSGDHAFGGGTEVVVK | |
| Variable Light Chain | | |
| ST-9V Second | QSLEESGGRLVTPGTPLTLTCTVSGIDLSRGQVGWVRQ | SEQ. ID. NO.: |
| Monoclonal Antibody | APGEGLEYIGFKGYGGNAFYTNWAKGRFTISKTSSTTV | 156 |
| (Rabbit) | DLKITTPTTEDTATYFCARVAGDINHLDLWGQGTLVTV | |
| Variable Heavy Chain | SS | |
| ST-9V First Monoclonal | AAVLTQTPSPVSAAVGGTVSINCQASKTVYDDNALAW | SEQ. ID. NO.: |
| Antibody | YQQKPGQPPKLLIYKASTLASGVPSRFSGSGSGTQFTLTI | 157 |
| (Mouse) | SDVQCDDAATYYCAGGYIYDSGDHAFGGGTEVVVKRA | |
| Full Length Light Chain | DAAPTVSIFPPSSEQLTSGGASVVCFLNNFYPKDINVKW | |
| | KIDGSERQNGVLNSWTDQDSKDSTYSMSSTLTLTKDEY | |
| | ERHNSYTCEATHKTSTSPIVKSFNRNEC | |
| ST-9V First Monoclonal | QSLEESGGRLVTPGTPLTLTCTVSGIDLSRGQVGWVRQ | SEQ. ID. NO.: |
| Antibody | APGEGLEYIGFKGYGGNAFYTNWAKGRFTISKTSSTTV | 158 |
| (Mouse) | DLKITTPTTEDTATYFCARVAGDINHLDLWGQGTLVTV | |
| Full Length Heavy | SSAKTTPPSVYPLAPGSAAQTNSMVTLGCLVKGYFPEP | |
| Chain | VTVTWNSGSLSSGVHTFPAVLQSDLYTLSSSVTVPSSPR | |
| | PSETVTCNVAHPASSTKVDKKIVPRDCGCKPCICTVPEV | |
| | SSVFIFPPKPKDVLTITLTPKVTCVVVDISKDDPEVQFSW | |
| | FVDDVEVHTAQTQPREEQFNSTFRSVSELPIMHQDWLN | |
| | GKEFKCRVNSAAFPAPIEKTISKTKGRPKAPQVYTIPPPK | |
| | EQMAKDKVSLTCMITDFFPEDITVEWQWNGQPAENYK | |
| | NTQPIMNTNGSYFVYSKLNVQKSNWEAGNTFTCSVLHE | |
| | GLHNHHTEKSLSHSPGK | |
| ST-9V Second | AAVLTQTPSPVSAAVGGTVSINCQASKTVYDDNALAW | SEQ. ID. NO.: |
| Monoclonal Antibody | YQQKPGQPPKLLIYKASTLASGVPSRFSGSGSGTQFTLTI | 159 |
| (Rabbit) | SDVQCDDAATYYCAGGYIYDSGDHAFGGGTEVVVKG | |
| Full Length Light Chain | DPVAPTVLIFPPAADQVATGTVTIVCVANKYFPDVTVT | |
| | WEVDGTTQTTGIENSKTPQNSADCTYNLSSTLTLTSTQY | |
| | NSHKEYTCKVTQGTTSVVQSFNRGDC | |
| ST-9V Second | QSLEESGGRLVTPGTPLTLTCTVSGIDLSRGQVGWVRQ | SEQ. ID. NO.: |
| Monoclonal Antibody | APGEGLEYIGFKGYGGNAFYTNWAKGRFTISKTSSTTV | 160 |
| (Rabbit) | DLKITTPTTEDTATYFCARVAGDINHLDLWGQGTLVTV | |
| Full Length Heavy | SSGQPKAPSVFPLAPCCGDTPSSTVTLGCLVKGYLPEPV | |
| Chain | TVTWNSGTLTNGVRTFPSVRQSSGLYSLSSVVSVTSSSQ | |
| | PVTCNVAHPATNTKVDKTVAPSTCSKPTCPPPELLGGPS | |
| | VFIFPPKPKDTLMISRTPEVTCVVVDVSQDDPEVQFTWY | |
| | INNEQVRTARPPLREQQFNSTIRVVSTLPIAHQDWLRGK | |
| | EFKCKVHNKALPAPIEKTISKARGQPLEPKVYTMGPPRE | |
| | ELSSRSVSLTCMINGFYPSDISVEWEKNGKAEDNYKTTP | |
| | AVLDSDGSYFLYSKLSVPTSEWQRGDVFTCSVMHEALH | |
| | NHYTQKSISRSPGK | |
| ST-14 First Monoclonal | RSSQSIVYSDGNTYLE | SEQ. ID. NO.: |
| Antibody (Mouse) | | 161 |
| Light Chain CDR 1 | | |
| ST-14 First Monoclonal | KVSHRFS | SEQ. ID. NO.: |
| Antibody | | 162 |
| (Mouse) | | |
| Light Chain CDR 2 | | |
| ST-14 First Monoclonal | FQGSHVPWT | SEQ. ID. NO.: |
| Antibody | | 163 |
| (Mouse) | | |
| Light Chain CDR 3 | | |
| ST-14 First Monoclonal | GYTFTDYYMK | SEQ. ID. NO.: |
| Antibody | | 164 |
| (Mouse) | | |
| Heavy Chain CDR 1 | | |
| ST-14 First Monoclonal | DINPNNYDTNYNQKFKG | SEQ. ID. NO.: |
| Antibody | | 165 |
| (Mouse) | | |
| Heavy Chain CDR 2 | | |
| ST-14 First Monoclonal | WDY | SEQ. ID. NO.: |
| Antibody | | 166 |
| (Mouse) | | |
| Heavy Chain CDR 3 | | |
| ST-14 Second | RSSQSIVYSDGNTYLE | SEQ. ID. NO.: |
| Monoclonal Antibody | | 167 |
| (Rabbit) | | |
| Light Chain CDR 1 | | |
| ST-14 Second | KVSHRFS | SEQ. ID. NO.: |
| Monoclonal Antibody | | 168 |
| (Rabbit) | | |
| Light Chain CDR 2 | | |
| ST-14 Second | FQGSHVPWT | SEQ. ID. NO.: |
| Monoclonal Antibody | | 169 |
| (Rabbit) | | |
| Light Chain CDR 3 | | |
| ST-14 Second | GYTFTDYYMK | SEQ. ID. NO.: |
| Monoclonal Antibody | | 170 |
| (Rabbit) | | |
| Heavy Chain CDR 1 | | |
| ST-14 Second | DINPNNYDTNYNQKFKG | SEQ. ID. NO.: |
| Monoclonal Antibody | | 171 |
| (Rabbit) | | |
| Heavy Chain CDR 2 | | |
| ST-14 Second | WDY | SEQ. ID. NO.: |
| Monoclonal Antibody | | 172 |
| (Rabbit) | | |
| Heavy Chain CDR 3 | | |
| ST-14 First Monoclonal | DVLMTQTPLSLPVSLGDQASIFCRSSQSIVYSDGNTYLE | SEQ. ID. NO.: |
| Antibody | WYLQKPGQSPKLLIYKVSHRFSGVPDRFSGSGSGTDFTL | 173 |
| (Mouse) | KISRVEAEDLGVYFCFQGSHVPWTFGGGTKLEIK | |
| Variable Light Chain | | |
| ST-14 First Monoclonal | EVQLQQSGPGLVKPGASVKMSCKASGYTFTDYYMKW | SEQ. ID. NO.: |
| Antibody | MKQSHGKSLEWIGDINPNNYDTNYNQKFKGRATLTVD | 174 |
| (Mouse) | KSSSTAYMQLNSLTSEDSAVYYCARWDYWGQGTTLTV | |
| Variable Heavy Chain | SS | |
| ST-14 Second | DVLMTQTPLSLPVSLGDQASIFCRSSQSIVYSDGNTYLE | SEQ. ID. NO.: |
| Monoclonal Antibody | WYLQKPGQSPKLLIYKVSHRFSGVPDRFSGSGSGTDFTL | 175 |
| (Rabbit) | KISRVEAEDLGVYFCFQGSHVPWTFGGGTKLEIK | |
| Variable Light Chain | | |
| ST-14 Second | EVQLQQSGPGLVKPGASVKMSCKASGYTFTDYYMKW | SEQ. ID. NO.: |
| Monoclonal Antibody | MKQSHGKSLEWIGDINPNNYDTNYNQKFKGRATLTVD | 176 |
| (Rabbit) | KSSSTAYMQLNSLTSEDSAVYYCARWDYWGQGTTLTV | |
| Variable Heavy Chain | SS | |
| ST-14 First Monoclonal | DVLMTQTPLSLPVSLGDQASIFCRSSQSIVYSDGNTYLE | SEQ. ID. NO.: |
| Antibody | WYLQKPGQSPKLLIYKVSHRFSGVPDRFSGSGSGTDFTL | 177 |
| (Mouse) | KISRVEAEDLGVYFCFQGSHVPWTFGGGTKLEIKRADA | |
| Full Length Light Chain | APTVSIFPPSSEQLTSGGASVVCFLNNFYPKDINVKWKID | |
| | GSERQNGVLNSWTDQDSKDSTYSMSSTLTLTKDEYERH | |
| | NSYTCEATHKTSTSPIVKSFNRNEC | |
| ST-14 First Monoclonal | EVQLQQSGPGLVKPGASVKMSCKASGYTFTDYYMKW | SEQ. ID. NO.: |
| Antibody | MKQSHGKSLEWIGDINPNNYDTNYNQKFKGRATLTVD | 178 |
| (Mouse) | KSSSTAYMQLNSLTSEDSAVYYCARWDYWGQGTTLTV | |
| Full Length Heavy | SSAKTTPPSVYPLAPGSAAQTNSMVTLGCLVKGYFPEP | |
| Chain | VTVTWNSGSLSSGVHTFPAVLQSDLYTLSSSVTVPSSTW | |
| | PSETVTCNVAHPASSTKVDKKIVPRDCGCKPCICTVPEV | |
| | SSVFIFPPKPKDVLTITLTPKVTCVVVDISKDDPEVQFSW | |
| | FVDDVEVHTAQTQPREEQFNSTFRSVSELPIMHQDWLN | |
| | GKEFKCRVNSAAFPAPIEKTISKTKGRPKAPQVYTIPPPK | |
| | EQMAKDKVSLTCMITDFFPEDITVEWQWNGQPAENYK | |
| | NTQPIMDTDGSYFVYSKLNVQKSNWEAGNTFTCSVLHE | |
| | GLHNHHTEKSLSHSPGK | |
| ST-14 Second | DVLMTQTPLSLPVSLGDQASIFCRSSQSIVYSDGNTYLE | SEQ. ID. NO.: |
| Monoclonal Antibody | WYLQKPGQSPKLLIYKVSHRFSGVPDRFSGSGSGTDFTL | 179 |
| (Rabbit) | KISRVEAEDLGVYFCFQGSHVPWTFGGGTKLEIKRDPV | |
| Full Length Light Chain | APTVLIFPPAADQVATGTVTIVCVANKYFPDVTVTWEV | |
| | DGTTQTTGIENSKTPQNSADCTYNLSSTLTLTSTQYNSH | |
| | KEYTCKVTQGTTSVVQSFNRGDC | |
| ST-14 Second | EVQLQQSGPGLVKPGASVKMSCKASGYTFTDYYMKW | SEQ. ID. NO.: |
| Monoclonal Antibody | MKQSHGKSLEWIGDINPNNYDTNYNQKFKGRATLTVD | 180 |
| (Rabbit) | KSSSTAYMQLNSLTSEDSAVYYCARWDYWGQGTTLTV | |
| Full Length Heavy | SSGQPKAPSVFPLAPCCGDTPSSTVTLGCLVKGYLPEPV | |
| Chain | TVTWNSGTLTNGVRTFPSVRQSSGLYSLSSVVSVTSSSQ | |
| | PVTCNVAHPATNTKVDKTVAPSTCSKPTCPPPELLGGPS | |
| | VFIFPPKPKDTLMISRTPEVTCVVVDVSEDDPEVQFTWY | |
| | INNEQVRTARPPLREQQFNSTIRVVSTLPIAHEDWLRGK | |
| | EFKCKVHNKALPAPIEKTISKARGQPLEPKVYTMGPPRE | |
| | ELSSRSVSLTCMINGFYPSDISVEWEKNGKAEDNYKTTP | |
| | AVLDSDGSYFLYSKLSVPTSEWQRGDVFTCSVM | |
| | HEALHNHYTQKSISRSPGK | |
| ST-18C First | RASQSIDNYLN | SEQ. ID. NO.: |
| Monoclonal Antibody | | 181 |
| (Human) | | |
| Light Chain CDR 1 | | |
| ST-18C First | AASSLQS | SEQ. ID. NO.: |
| Monoclonal Antibody | | 182 |
| (Human) | | |
| Light Chain CDR 2 | | |
| ST-18C First | QQSYSSPYT | SEQ. ID. NO.: |
| Monoclonal Antibody | | 183 |
| (Human) | | |
| Light Chain CDR 3 | | |
| ST-18C First | GLTFSNSWMS | SEQ. ID. NO.: |
| Monoclonal Antibody | | 184 |
| (Human) | | |
| Heavy Chain CDR 1 | | |
| ST-18C First | NINQDAGQKYSVDSVRG | SEQ. ID. NO.: |
| Monoclonal Antibody | | 185 |
| (Human) | | |
| Heavy Chain CDR 2 | | |
| ST-18C First | LGGWRHLDY | SEQ. ID. NO.: |
| Monoclonal Antibody | | 186 |
| (Human) | | |
| Heavy Chain CDR 3 | | |
| ST-18C Second | RASQSIDNYLN | SEQ. ID. NO.: |
| Monoclonal Antibody | | 187 |
| (Rabbit) | | |
| Light Chain CDR 1 | | |
| ST-18C Second | AASSLQS | SEQ. ID. NO.: |
| Monoclonal Antibody | | 188 |
| (Rabbit) | | |
| Light Chain CDR 2 | | |
| ST-18C Second | QQSYSSPYT | SEQ. ID. NO.: |
| Monoclonal Antibody | | 189 |
| (Rabbit) | | |
| Light Chain CDR 3 | | |
| ST-18C Second | GLTFSNSWMS | SEQ. ID. NO.: |
| Monoclonal Antibody | | 190 |
| (Rabbit) | | |
| Heavy Chain CDR 1 | | |
| ST-18C Second | NINQDAGQKYSVDSVRG | SEQ. ID. NO.: |
| Monoclonal Antibody | | 191 |
| (Rabbit) | | |
| Heavy Chain CDR 2 | | |
| ST-18C Second | LGGWRHLDY | SEQ. ID. NO.: |
| Monoclonal Antibody | | 192 |
| (Rabbit) | | |
| Heavy Chain CDR 3 | | |
| ST-18C First | DIQMTQSPSSLSASVGDRVTITCRASQSIDNYLNWYQQK | SEQ. ID. NO.: |
| Monoclonal Antibody | SGKAPDLLIYAASSLQSGVPSRFSGSGSGTDFTLTISSLQ | 193 |
| (Human) | PEDFATYYCQQSYSSPYTFGQGTKLEIK | |
| Variable Light Chain | | |
| ST-18C First | EVQLVESGGGLVQPGGSLRLSCAASGLTFSNSWMSWV | SEQ. ID. NO.: |
| Monoclonal Antibody | RQTPGKGLEWVANINQDAGQKYSVDSVRGRFTISRDN | 194 |
| (Human) | AKNSLYLQMNSLRAEDTAVYYCARLGGWRHLDYWGQ | |
| Variable Heavy Chain | GTLVTVSS | |
| ST-18C Second | DIQMTQSPSSLSASVGDRVTITCRASQSIDNYLNWYQQK | SEQ. ID. NO.: |
| Monoclonal Antibody | SGKAPDLLIYAASSLQSGVPSRFSGSGSGTDFTLTISSLQ | 195 |
| (Rabbit) | PEDFATYYCQQSYSSPYTFGQGTKLEIK | |
| Variable Light Chain | | |
| ST-18C Second | EVQLVESGGGLVQPGGSLRLSCAASGLTFSNSWMSWV | SEQ. ID. NO.: |
| Monoclonal Antibody | RQTPGKGLEWVANINQDAGQKYSVDSVRGRFTISRDN | 196 |
| (Rabbit) | AKNSLYLQMNSLRAEDTAVYYCARLGGWRHLDYWGQ | |
| Variable Heavy Chain | GTLVTVSS | |
| ST-18C First | DIQMTQSPSSLSASVGDRVTITCRASQSIDNYLNWYQQK | SEQ. ID. NO.: |
| Monoclonal Antibody | SGKAPDLLIYAASSLQSGVPSRFSGSGSGTDFTLTISSLQ | 197 |
| (Human) | PEDFATYYCQQSYSSPYTFGQGTKLEIKRTVAAPSVFIFP | |
| Full Length Light Chain | PSDEQLKSGTASVVCLLNNFYPREAKVQWKVDNALQS | |
| | GNSQESVTEQDSKDSTYSLSSTLTLSKADYEKHKVYAC | |
| | EVTHQGLSSPVTKSFNRGEC | |
| ST-18C First | EVQLVESGGGLVQPGGSLRLSCAASGLTFSNSWMSWV | SEQ. ID. NO.: |
| Monoclonal Antibody | RQTPGKGLEWVANINQDAGQKYSVDSVRGRFTISRDN | 198 |
| (Human) | AKNSLYLQMNSLRAEDTAVYYCARLGGWRHLDYWGQ | |
| Full Length Heavy | GTLVTVSSASTKGPSVFPLAPSSKSTSGGTAALGCLVKD | |
| Chain | YFPEPVTVSWNSGALTSGVHTFPAVLQSSGLYSLSSVVT | |
| | VPSSSLGTQTYICNVNHKPSNTKVDKKVEPKSCDKTHT | |
| | CPPCPAPELLGGPSVFLFPPKPKDTLMISRTPEVTCVVVD | |
| | VSHEDPEVKFNWYVDGVEVHNAKTKPREEQYNSTYRV | |
| | VSVLTVLHQDWLNGKEYKCKVSNKALPAPIEKTISKAK | |
| | GQPREPQVYTLPPSRDELTKNQVSLTCLVKGFYPSDIAV | |
| | EWESNGQPENNYKTTPPVLDSDGSFFLYSKLTVDKSRW | |
| | QQGNVFSCSVMHEALHNHYTQKSLSLSPGK | |
| ST-18C Second | DIQMTQSPSSLSASVGDRVTITCRASQSIDNYLNWYQQK | SEQ. ID. NO.: |
| Monoclonal Antibody | SGKAPDLLIYAASSLQSGVPSRFSGSGSGTDFTLTISSLQ | 199 |
| (Rabbit) | PEDFATYYCQQSYSSPYTFGQGTKLEIKGDPVAPTVLIFP | |
| Full Length Light Chain | PAADQVATGTVTIVCVANKYFPDVTVTWEVDGTTQTT | |
| | GIENSKTPQNSADCTYNLSSTLTLTSTQYNSHKEYTCKV | |
| | TQGTTSVVQSFNRGDC | |
| ST-18C Second | EVQLVESGGGLVQPGGSLRLSCAASGLTFSNSWMSWV | SEQ. ID. NO.: |
| Monoclonal Antibody | RQTPGKGLEWVANINQDAGQKYSVDSVRGRFTISRDN | 200 |
| (Rabbit) | AKNSLYLQMNSLRAEDTAVYYCARLGGWRHLDYWGQ | |
| Full Length Heavy | GTLVTVSSGQPKAPSVFPLAPCCGDTPSSTVTLGCLVKG | |
| Chain | YLPEPVTVTWNSGTLTNGVRTFPSVRQSSGLYSLSSVVS | |
| | VTSSSQPVTCNVAHPATNTKVDKTVAPSTCSKPTCPPPE | |
| | LLGGPSVFIFPPKPKDTLMISRTPEVTCVVVDVSQDDPE | |
| | VQFTWYINNEQVRTARPPLREQQFNSTIRVVSTLPIAHQ | |
| | DWLRGKEFKCKVHNKALPAPIEKTISKARGQPLEPKVY | |
| | TMGPPREELSSRSVSLTCMINGFYPSDISVEWEKNGKAE | |
| | DNYKTTPAVLDSDGSYFLYSKLSVPTSEWQRGDVFTCS | |
| | VMHEALHNHYTQKSISRSPGK | |
| ST-19A First | QASQSISSYLA | SEQ. ID. NO.: |
| Monoclonal Antibody | | 201 |
| (Mouse or Human) | | |
| Light Chain CDR 1 | | |
| ST-19A First | RASTLTS | SEQ. ID. NO.: |
| Monoclonal Antibody | | 202 |
| (Human) | | |
| Light Chain CDR 2 | | |
| ST-19A First | QCTGGEICVKGRT | SEQ. ID. NO.: |
| Monoclonal Antibody | | 203 |
| (Human) | | |
| Light Chain CDR 3 | | |
| ST-19A First | GFSFSGVYDIC | SEQ. ID. NO.: |
| Monoclonal Antibody | | 204 |
| (Human) | | |
| Heavy Chain CDR 1 | | |
| ST-19A First | CIYIVTANPYYATWAKG | SEQ. ID. NO.: |
| Monoclonal Antibody | | 205 |
| (Human) | | |
| Heavy Chain CDR 2 | | |
| ST-19A First | GGDSTAWSWEL | SEQ. ID. NO.: |
| Monoclonal Antibody | | 206 |
| (Human) | | |
| Heavy Chain CDR 3 | | |
| ST-19A Second | QASQSISSYLA | SEQ. ID. NO.: |
| Monoclonal Antibody | | 207 |
| (Rabbit) | | |
| Light Chain CDR 1 | | |
| ST-19A Second | RASTLTS | SEQ. ID. NO.: |
| Monoclonal Antibody | | 208 |
| (Rabbit) | | |
| Light Chain CDR 2 | | |
| ST-19A Second | QCTGGEICVKGRT | SEQ. ID. NO.: |
| Monoclonal Antibody | | 209 |
| (Rabbit) | | |
| Light Chain CDR 3 | | |
| ST-19A Second | GFSFSGVYDIC | SEQ. ID. NO.: |
| Monoclonal Antibody | | 210 |
| (Rabbit) | | |
| Heavy Chain CDR 1 | | |
| ST-19A Second | CIYIVTANPYYATWAKG | SEQ. ID. NO.: |
| Monoclonal Antibody | | 211 |
| (Rabbit) | | |
| Heavy Chain CDR 2 | | |
| ST-19A Second | GGDSTAWSWEL | SEQ. ID. NO.: |
| Monoclonal Antibody | | 212 |
| (Rabbit) | | |
| Heavy Chain CDR 3 | | |
| ST-19A First | AFELTQTPASVEAAVGGTVTIKCQASQSISSYLAWYQQ | SEQ. ID. NO.: |
| Monoclonal Antibody | KPGQPPKLLIYRASTLTSGVPSRFRGSGSGTEFTLTISDLE | 213 |
| (Human) | CADAATYYCQCTGGEICVKGRTFGGGTEVVVE | |
| Variable Light Chain | | |
| ST-19A First | QEQLEESGGGLVQPEGSLTLTCTASGFSFSGVYDICWVR | SEQ. ID. NO.: |
| Monoclonal Antibody | QAPGKGPEWIACIYIVTANPYYATWAKGRFTMSKTSST | 214 |
| (Human) | TVTLQLNSLTAADTATYFCVRGGDSTAWSWELWGPGT | |
| Variable Heavy Chain | LVTVSS | |
| ST-19A Second | AFELTQTPASVEAAVGGTVTIKCQASQSISSYLAWYQQ | SEQ. ID. NO.: |
| Monoclonal Antibody | KPGQPPKLLIYRASTLTSGVPSRFRGSGSGTEFTLTISDLE | 215 |
| (Rabbit) | CADAATYYCQCTGGEICVKGRTFGGGTEVVVE | |
| Variable Light Chain | | |
| ST-19A Second | QEQLEESGGGLVQPEGSLTLTCTASGFSFSGVYDICWVR | SEQ. ID. NO.: |
| Monoclonal Antibody | QAPGKGPEWIACIYIVTANPYYATWAKGRFTMSKTSST | 216 |
| (Rabbit) | TVTLQLNSLTAADTATYFCVRGGDSTAWSWELWGPGT | |
| Variable Heavy Chain | LVTVSS | |
| ST-19A First | AFELTQTPASVEAAVGGTVTIKCQASQSISSYLAWYQQ | SEQ. ID. NO.: |
| Monoclonal Antibody | KPGQPPKLLIYRASTLTSGVPSRFRGSGSGTEFTLTISDLE | 217 |
| (Human) | CADAATYYCQCTGGEICVKGRTFGGGTEVVVERTVAA | |
| Full Length Light Chain | PSVFIFPPSDEQLKSGTASVVCLLNNFYPREAKVQWKV | |
| | DNALQSGNSQESVTEQDSKDSTYSLSSTLTLSKADYEK | |
| | HKVYACEVTHQGLSSPVTKSFNRGEC | |
| ST-19A First | QEQLEESGGGLVQPEGSLTLTCTASGFSFSGVYDICWVR | SEQ. ID. NO.: |
| Monoclonal Antibody | QAPGKGPEWIACIYIVTANPYYATWAKGRFTMSKTSST | 218 |
| (Human) | TVTLQLNSLTAADTATYFCVRGGDSTAWSWELWGPGT | |
| Full Length Heavy | LVTVSSASTKGPSVFPLAPSSKSTSGGTAALGCLVKDYF | |
| Chain | PEPVTVSWNSGALTSGVHTFPAVLQSSGLYSLSSVVTVP | |
| | SSSLGTQTYICNVNHKPSNTKVDKKVEPKSCDKTHTCPP | |
| | CPAPELLGGPSVFLFPPKPKDTLMISRTPEVTCVVVDVS | |
| | HEDPEVKFNWYVDGVEVHNAKTKPREEQYNSTYRVVS | |
| | VLTVLHQDWLNGKEYKCKVSNKALPAPIEKTISKAKGQ | |
| | PREPQVYTLPPSRDELTKNQVSLTCLVKGFYPSDIAVEW | |
| | ESNGQPENNYKTTPPVLDSDGSFFLYSKLTVDKSRWQQ | |
| | GNVFSCSVMHEALHNHYTQKSLSLSPGK | |
| ST-19A Second | AFELTQTPASVEAAVGGTVTIKCQASQSISSYLAWYQQ | SEQ. ID. NO.: |
| Monoclonal Antibody | KPGQPPKLLIYRASTLTSGVPSRFRGSGSGTEFTLTISDLE | 219 |
| (Rabbit) | CADAATYYCQCTGGEICVKGRTFGGGTEVVVEGDPVA | |
| Full Length Light Chain | PTVLIFPPAADQVATGTVTIVCVANKYFPDVTVTWEVD | |
| | GTTQQSGIENSKTPQNSADCTYNLSSTLTLTSTQYNSHK | |
| | EYTCKVTQGTTSVVQSFNRGDC | |
| ST-19A Second | QEQLEESGGGLVQPEGSLTLTCTASGFSFSGVYDICWVR | SEQ. ID. NO.: |
| Monoclonal Antibody | QAPGKGPEWIACIYIVTANPYYATWAKGRFTMSKTSST | 220 |
| (Rabbit) | TVTLQLNSLTAADTATYFCVRGGDSTAWSWELWGPGT | |
| Full Length Heavy | LVTVSSGQPKAPSVFPLAPCCGDTPSSTVTLGCLVKGYL | |
| Chain | PEPVTVTWNSGTLTNGVRTFPSVRQSSGLYSLSSVVSVT | |
| | SSSQPVTCNVAHPATNTKVDKTVAPSTCSKPMCPPPELL | |
| | GGPSVFIFPPKPKDTLMISRTPEVTCVVVDVSQDDPEVQ | |
| | FTWYINNEQVRTARPPLREQQFNSTIRVVSTLPIAHQDW | |
| | LRGKEFKCKVHNKALPAPIEKTISKARGQPLEPKVYTM | |
| | GPPREELSSRSVSLTCMINGFYPSDISVEWEKNGKAEDN | |
| | YKTTPTVLDSDGSYFLYSKLSVPTSEWQRGDVFTCSVM | |
| | HEALHNHYTQKSISRSPGK | |
| ST-19F First | QSSQSVYSNNLLS | SEQ. ID. NO.: |
| Monoclonal Antibody | | 221 |
| (Human) | | |
| Light Chain CDR 1 | | |
| ST-19F First | RASTLAS | SEQ. ID. NO.: |
| Monoclonal Antibody | | 222 |
| (Human) | | |
| Light Chain CDR 2 | | |
| ST-19F First | GAVYSGYIYG | SEQ. ID. NO.: |
| Monoclonal Antibody | | 223 |
| (Human) | | |
| Light Chain CDR 3 | | |
| ST-19F First | GFSLITYTVI | SEQ. ID. NO.: |
| Monoclonal Antibody | | 224 |
| (Human) | | |
| Heavy Chain CDR 1 | | |
| ST-19F First | AIRSGESISYASWA KG | SEQ. ID. NO.: |
| Monoclonal Antibody | | 225 |
| (Human) | | |
| Heavy Chain CDR 2 | | |
| ST-19F First | SGNGFDS | SEQ. ID. NO.: |
| Monoclonal Antibody | | 226 |
| (Human) | | |
| Heavy Chain CDR 3 | | |
| ST-19F Second | QSSQSVYSNNLLS | SEQ. ID. NO.: |
| Monoclonal Antibody | | 227 |
| (Rabbit) | | |
| Light Chain CDR 1 | | |
| ST-19F Second | RASTLAS | SEQ. ID. NO.: |
| Monoclonal Antibody | | 228 |
| (Rabbit) | | |
| Light Chain CDR 2 | | |
| ST-19F Second | GAVYSGYIYG | SEQ. ID. NO.: |
| Monoclonal Antibody | | 229 |
| (Rabbit) | | |
| Light Chain CDR 3 | | |
| ST-19F Second | GFSLITYTVI | SEQ. ID. NO.: |
| Monoclonal Antibody | | 230 |
| (Rabbit) | | |
| Heavy Chain CDR 1 | | |
| ST-19F Second | AIRSGESISYASWAKG | SEQ. ID. NO.: |
| Monoclonal Antibody | | 231 |
| (Rabbit) | | |
| Heavy Chain CDR 2 | | |
| ST-19F Second | SGNGFDS | SEQ. ID. NO.: |
| Monoclonal Antibody | | 232 |
| (Rabbit) | | |
| Heavy Chain CDR 3 | | |
| ST-19F First | DPVMTQTPSSTSAAVGGTVTINCQSSQSVYSNNLLSWY | SEQ. ID. NO.: |
| Monoclonal Antibody | QQKPGQPPKLLIYRASTLASGVPSRFKGSGSGTQFSLTIS | 233 |
| (Human) | DLECDDVATYYCGAVYSGYIYGFGGGTEVVVK | |
| Variable Light Chain | | |
| ST-19F First | QSVEESGGRLVTPGTPLTLTCTVSGFSLITYTVIWVRQA | SEQ. ID. NO.: |
| Monoclonal Antibody | PGKGLEWIGAIRSGESISYASWAKGRFTISETSTTVDLKI | 234 |
| (Human) | TSPTTEDTATYFCTRSGNGFDSWGPGTLVTVSS | |
| Variable Heavy Chain | | |
| ST-19F Second | DPVMTQTPSSTSAAVGGTVTINCQSSQSVYSNNLLSWY | SEQ. ID. NO.: |
| Monoclonal Antibody | QQKPGQPPKLLIYRASTLASGVPSRFKGSGSGTQFSLTIS | 235 |
| (Rabbit) | DLECDDVATYYCGAVYSGYIYGFGGGTEVVVK | |
| Variable Light Chain | | |
| ST-19F Second | QSVEESGGRLVTPGTPLTLTCTVSGFSLITYTVIWVRQA | SEQ. ID. NO.: |
| Monoclonal Antibody | PGKGLEWIGAIRSGESISYASWAKGRFTISETSTTVDLKI | 236 |
| (Rabbit) | TSPTTEDTATYFCTRSGNGFDSWGPGTLVTVSS | |
| Variable Heavy Chain | | |
| ST-19F First | DPVMTQTPSSTSAAVGGTVTINCQSSQSVYSNNLLSWY | SEQ. ID. NO.: |
| Monoclonal Antibody | QQKPGQPPKLLIYRASTLASGVPSRFKGSGSGTQFSLTIS | 237 |
| (Human) | DLECDDVATYYCGAVYSGYIYGFGGGTEVVVKRTVAA | |
| Full Length Light Chain | PSVFIFPPSDEQLKSGTASVVCLLNNFYPREAKVQWKV | |
| | DNALQSGNSQESVTEQDSKDSTYSLSSTLTLSKADYEK | |
| | HKVYACEVTHQGLSSPVTKSFNRGEC | |
| ST-19F First | QSVEESGGRLVTPGTPLTLTCTVSGFSLITYTVIWVRQA | SEQ. ID. NO.: |
| Monoclonal Antibody | PGKGLEWIGAIRSGESISYASWAKGRFTISETSTTVDLKI | 238 |
| (Human) | TSPTTEDTATYFCTRSGNGFDSWGPGTLVTVSSASTKGP | |
| Full Length Heavy | SVFPLAPSSKSTSGGTAALGCLVKDYFPEPVTVSWNSG | |
| Chain | ALTSGVHTFPAVLQSSGLYSLSSVVTVPSSSLGTQTYICN | |
| | VNHKPSNTKVDKKVEPKSCDKTHTCPPCPAPELLGGPS | |
| | VFLFPPKPKDTLMISRTPEVTCVVVDVSHEDPEVKFNW | |
| | YVDGVEVHNAKTKPREEQYNSTYRVVSVLTVLHQDWL | |
| | NGKEYKCKVSNKALPAPIEKTISKAKGQPREPQVYTLPP | |
| | SRDELTKNQVSLTCLVKGFYPSDIAVEWESNGQPENNY | |
| | KTTPPVLDSDGSFFLYSKLTVDKSRWQQGNVFSCSVMH | |
| | EALHNHYTQKSLSLSPGK | |
| ST-19F Second | DPVMTQTPSSTSAAVGGTVTINCQSSQSVYSNNLLSWY | SEQ. ID. NO.: |
| Monoclonal Antibody | QQKPGQPPKLLIYRASTLASGVPSRFKGSGSGTQFSLTIS | 239 |
| (Rabbit) | DLECDDVATYYCGAVYSGYIYGFGGGTEVVVKGDPVA | |
| Full Length Light Chain | PTVLIFPPAADQVATGTVTIVCVANKYFPDVTVTWEVD | |
| | GTTQTTGIENSKTPQNSADCTYNLSSTLTLTSTQYNSHK | |
| | EYTCKVTQGTTSVVQSFNRGDC | |
| ST-19F Second | QSVEESGGRLVTPGTPLTLTCTVSGFSLITYTVIWVRQA | SEQ. ID. NO.: |
| Monoclonal Antibody | PGKGLEWIGAIRSGESISYASWAKGRFTISETSTTVDLKI | 240 |
| (Rabbit) | TSPTTEDTATYFCTRSGNGFDSWGPGTLVTVSSGQPKAP | |
| Full Length Heavy | SVFPLAPCCGDTPSSTVTLGCLVKGYLPEPVTVTWNSGT | |
| Chain | LTNGVRTFPSVRQSSGLYSLSSVVSVTSSSQPVTCNVAH | |
| | PATNTKVDKTVAPSTCSKPTCPPPELLGGPSVFIFPPKPK | |
| | DTLMISRTPEVTCVVVDVSQDDPEVQFTWYINNEQVRT | |
| | ARPPLREQQFNSTIRVVSTLPIA | |
| | HQDWLRGKEFKCKVHNKALPAPIEKTISKARGQPLEPK | |
| | VYTMGPPREELSSRSVSLTCMINGFYPSDISVEWEKNGK | |
| | AEDNYKTTPAVLDSDGSYFLYSKLSVPTSEWQRGDVFT | |
| | CSVMHEALHNHYTQKSISRSPGK | |
| ST-22F First | QASESVGDRLA | SEQ. ID. NO.: |
| Monoclonal Antibody | | 241 |
| (Human) | | |
| Light Chain CDR 1 | | |
| ST-22F First | YIATLAS | SEQ. ID. NO.: |
| Monoclonal Antibody | | 242 |
| (Human) | | |
| Light Chain CDR 2 | | |
| ST-22F First | QYTNYDSVSGNV | SEQ. ID. NO.: |
| Monoclonal Antibody | | 243 |
| (Human) | | |
| Light Chain CDR 3 | | |
| ST-22F First | GIDFSGYVYMC | SEQ. ID. NO.: |
| Monoclonal Antibody | | 244 |
| (Human) | | |
| Heavy Chain CDR 1 | | |
| ST-22F First | CIDTDGSGRTWYASWAKG | SEQ. ID. NO.: |
| Monoclonal Antibody | | 245 |
| (Human) | | |
| Heavy Chain CDR 2 | | |
| ST-22F First | ETRSTGFSFNL | SEQ. ID. NO.: |
| Monoclonal Antibody | | 246 |
| (Human) | | |
| Heavy Chain CDR 3 | | |
| ST-22F Second | QASESVGDRLA | SEQ. ID. NO.: |
| Monoclonal Antibody | | 247 |
| (Rabbit) | | |
| Light Chain CDR 1 | | |
| ST-22F Second | YIATLAS | SEQ. ID. NO.: |
| Monoclonal Antibody | | 248 |
| (Rabbit) | | |
| Light Chain CDR 2 | | |
| ST-22F Second | QYTNYDSVSGNV | SEQ. ID. NO.: |
| Monoclonal Antibody | | 249 |
| (Rabbit) | | |
| Light Chain CDR 3 | | |
| ST-22F Second | GIDFSGYVYMC | SEQ. ID. NO.: |
| Monoclonal Antibody | | 250 |
| (Rabbit) | | |
| Heavy Chain CDR 1 | | |
| ST-22F Second | CIDTDGSGRTWYASWAKG | SEQ. ID. NO.: |
| Monoclonal Antibody | | 251 |
| (Rabbit) | | |
| Heavy Chain CDR 2 | | |
| ST-22F Second | ETRSTGFSFNL | SEQ. ID. NO.: |
| Monoclonal Antibody | | 252 |
| (Rabbit) | | |
| Heavy Chain CDR 3 | | |
| ST-22F First | DIVMTQTPASVSAAVGGTVTIKCQASESVGDRLAWYQ | SEQ. ID. NO.: |
| Monoclonal Antibody | QKPGQPPKLLIYYIATLASGVSSRFKGSGSGTQFTLTISD | 253 |
| (Human) | LECDDAATYYCQYTNYDSVSGNVFGGGTEVVVR | |
| Variable Light Chain | | |
| ST-22F First | QSLEESGGDLVKPGASLTLTCTASGIDFSGYVYMCWVR | SEQ. ID. NO.: |
| Monoclonal Antibody | QAPGKGLEWITCIDTDGSGRTWYASWAKGRFTISKTST | 254 |
| (Human) | TVTLQMASLTAADTATYFCARETRSTGFSFNWGPGTLV | |
| Variable Heavy Chain | TVSS | |
| ST-22F Second | DIVMTQTPASVSAAVGGTVTIKCQASESVGDRLAWYQ | SEQ. ID. NO.: |
| Monoclonal Antibody | QKPGQPPKLLIYYIATLASGVSSRFKGSGSGTQFTLTISD | 255 |
| (Rabbit) | LECDDAATYYCQYTNYDSVSGNVFGGGTEVVVR | |
| Variable Light Chain | | |
| ST-22F Second | QSLEESGGDLVKPGASLTLTCTASGIDFSGYVYMCWVR | SEQ. ID. NO.: |
| Monoclonal Antibody | QAPGKGLEWITCIDTDGSGRTWYASWAKGRFTISKTST | 256 |
| (Rabbit) | TVTLQMASLTAADTATYFCARETRSTGFSFNLWGPGTL | |
| Variable Heavy Chain | VTVSS | |
| ST-22F First | DIVMTQTPASVSAAVGGTVTIKCQASESVGDRLAWYQ | SEQ. ID. NO.: |
| Monoclonal Antibody | QKPGQPPKLLIYYIATLASGVSSRFKGSGSGTQFTLTISD | 257 |
| (Human) | LECDDAATYYCQYTNYDSVSGNVFGGGTEVVVRRTVA | |
| Full Length Light Chain | APSVFIFPPSDEQLKSGTASVVCLLNNFYPREAKVQWK | |
| | VDNALQSGNSQESVTEQDSKDSTYSLSSTLTLSKADYE | |
| | KHKVYACEVTHQGLSSPVTKSFNRGEC | |
| ST-22F First | QSLEESGGDLVKPGASLTLTCTASGIDFSGYVYMCWVR | SEQ. ID. NO.: |
| Monoclonal Antibody | QAPGKGLEWITCIDTDGSGRTWYASWAKGRFTISKTST | 258 |
| (Human) | TVTLQMASLTAADTATYFCARETRSTGFSFNLWGPGTL | |
| Full Length Heavy | VTVSSASTKGPSVFPLAPSSKSTSGGTAAL | |
| Chain | GCLVKDYFPEPVTVSWNSGALTSGVHTFPAVLQSSGLY | |
| | SLSSVVTVPSSSLGTQTYICNVNHKPSNTKVDKKVEPKS | |
| | CDKTHTCPPCPAPELLGGPSVFLFPPKPKDTLMISRTPEV | |
| | TCVVVDVSHEDPEVKFNWYVDGVEVHNAKTKPREEQ | |
| | YNSTYRVVSVLTVLHQDWLNGKEYKCKVSNKALPAPI | |
| | EKTISKAKGQPREPQVYTLPPSRDELTKNQVSLTCLVKG | |
| | FYPSDIAVEWESNGQPENNYKTTPPVLDSDGSFFLYSKL | |
| | TVDKSRWQQGNVFSCSVMHEALHNHYTQKSLSLSPGK | |
| ST-22F Second | DIVMTQTPASVSAAVGGTVTIKCQASESVGDRLAWYQ | SEQ. ID. NO.: |
| Monoclonal Antibody | QKPGQPPKLLIYYIATLASGVSSRFKGSGSGTQFTLTISD | 259 |
| (Rabbit) | LECDDAATYYCQYTNYDSVSGNVFGGGTEVVVRGDPV | |
| Full Length Light Chain | APTVLIFPPAADQVATGTVTIVCVANKYFPDVTVTWEV | |
| | DGTTQTTGIENSKTPQNSADCTYNLSSTLTLTSTQYNSH | |
| | KEYTCKVTQGTTSVVQSFNRGDC | |
| ST-22F Second | QSLEESGGDLVKPGASLTLTCTASGIDFSGYVYMCWVR | SEQ. ID. NO.: |
| Monoclonal Antibody | QAPGKGLEWITCIDTDGSGRTWYASWAKGRFTISKTST | 260 |
| (Rabbit) | TVTLQMASLTAADTATYFCARETRSTGFSFNLWGPGTL | |
| Full Length Heavy | VTVSSGQPKAPSVFPLAPCCGDTPSSTVTLGCLVKGYLP | |
| Chain | EPVTVTWNSGTLTNGVRTFPSVRQSSGLYSLSSVVSVTS | |
| | SSQPVTCNVAHPATNTKVDKTVAPSTCSKPTCPPPELLG | |
| | GPSVFIFPPKPKDTLMISRTPEVTCVVVDVSQDDPEVQF | |
| | TWYINNEQVRTARPPLREQQFNSTIRVVSTLPIAHQDWL | |
| | RGKEFKCKVHNKALPAPIEKTISKARGQPLEPKVYTMG | |
| | PPREELSSRSVSLTCMINGFYPSDISVEWEKNGKAEDNY | |
| | KTTPAVLDSDGSYFLYSKLSVPTSEWQRGDVFTCSVMH | |
| | EALHNHYTQKSISRSPGK | |
| ST-23F First | QASQSVVDNNWLA | SEQ. ID. NO.: |
| Monoclonal Antibody | | 261 |
| (Human) | | |
| Light Chain CDR 1 | | |
| ST-23F First | RASNLAA | SEQ. ID. NO.: |
| Monoclonal Antibody | | 262 |
| (Human) | | |
| Light Chain CDR 2 | | |
| ST-23F First | GAHVSFGIFG | SEQ. ID. NO.: |
| Monoclonal Antibody | | 263 |
| (Human) | | |
| Light Chain CDR 3 | | |
| ST-23F First | GFSLSNHGVN | SEQ. ID. NO.: |
| Monoclonal Antibody | | 264 |
| (Human) | | |
| Heavy Chain CDR 1 | | |
| ST-23F First | IINGNGGTWYASWAKG | SEQ. ID. NO.: |
| Monoclonal Antibody | | 265 |
| (Human) | | |
| Heavy Chain CDR 2 | | |
| ST-23F First | GGAGAADSDI | SEQ. ID. NO.: |
| Monoclonal Antibody | | 266 |
| (Human) | | |
| Heavy Chain CDR 3 | | |
| ST-23F Second | QASQSVVDNNWLA | SEQ. ID. NO.: |
| Monoclonal Antibody | | 267 |
| (Rabbit) | | |
| Light Chain CDR 1 | | |
| ST-23F Second | RASNLAA | SEQ. ID. NO.: |
| Monoclonal Antibody | | 268 |
| (Rabbit) | | |
| Light Chain CDR 2 | | |
| ST-23F Second | GAHVSFGIFG | SEQ. ID. NO.: |
| Monoclonal Antibody | | 269 |
| (Rabbit) | | |
| Light Chain CDR 3 | | |
| ST-23F Second | GFSLSNHGVN | SEQ. ID. NO.: |
| Monoclonal Antibody | | 270 |
| (Rabbit) | | |
| Heavy Chain CDR 1 | | |
| ST-23F Second | IINGNGGTWYASWAKG | SEQ. ID. NO.: |
| Monoclonal Antibody | | 271 |
| (Rabbit) | | |
| Heavy Chain CDR 2 | | |
| ST-23F Second | GGAGAADSDI | SEQ. ID. NO.: |
| Monoclonal Antibody | | 272 |
| (Rabbit) | | |
| Heavy Chain CDR 3 | | |
| ST-23F First | AQVLTQTPSSTSAAVGGTVTINCQASQSVVDNNWLAW | SEQ. ID. NO.: |
| Monoclonal Antibody | YQQKPGQPPKQLIYRASNLAAGVPSRFKGSGSGPQFTLT | 273 |
| (Human) | ISDLECDDAATYFCGAHVSFGIFGFGGGTEVVVK | |
| Variable Light Chain | | |
| ST-23F First | QSVEESGGRLVTPGTPLTLTCTVSGFSLSNHGVNWVRQ | SEQ. ID. NO.: |
| Monoclonal Antibody | APGKGLEWIGIINGNGGTWYASWAKGRFTISKTSTTVD | 274 |
| (Human) | LKMTSPTTEDTATYFCARGGAGAADSDIWGPGTLVTVS | |
| Variable Heavy Chain | L | |
| ST-23F Second | AQVLTQTPSSTSAAVGGTVTINCQASQSVVDNNWLAW | SEQ. ID. NO.: |
| Monoclonal Antibody | YQQKPGQPPKQLIYRASNLAAGVPSRFKGSGSGPQFTLT | 275 |
| (Rabbit) | ISDLECDDAATYFCGAHVSFGIFGFGGGTEVVVK | |
| Variable Light Chain | | |
| ST-23F Second | QSVEESGGRLVTPGTPLTLTCTVSGFSLSNHGVNWVRQ | SEQ. ID. NO.: |
| Monoclonal Antibody | APGKGLEWIGIINGNGGTWYASWAKGRFTISKTSTTVD | 276 |
| (Rabbit) | LKMTSPTTEDTATYFCARGGAGAADSDIWGPGTLVTVS | |
| Variable Heavy Chain | L | |
| ST-23F First | AQVLTQTPSSTSAAVGGTVTINCQASQSVVDNNWLAW | SEQ. ID. NO.: |
| Monoclonal Antibody | YQQKPGQPPKQLIYRASNLAAGVPSRFKGSGSGPQFTLT | 277 |
| (Human) | ISDLECDDAATYFCGAHVSFGIFGFGGGTEVVVKRTVA | |
| Full Length Light Chain | APSVFIFPPSDEQLKSGTASVVCLLNNFYPREAKVQWK | |
| | VDNALQSGNSQESVTEQDSKDSTYSLSSTLTLSKADYE | |
| | KHKVYACEVTHQGLSSPVTKSFNRGEC | |
| ST-23F First | QSVEESGGRLVTPGTPLTLTCTVSGFSLSNHGVNWVRQ | SEQ. ID. NO.: |
| Monoclonal Antibody | APGKGLEWIGIINGNGGTWYASWAKGRFTISKTSTTVD | 278 |
| (Human) | LKMTSPTTEDTATYFCARGGAGAADSDIWGPGTLVTVS | |
| Full Length Heavy | LASTKGPSVFPLAPSSKSTSGGTAALGCLVKDYFPEPVT | |
| Chain | VSWNSGALTSGVHTFPAVLQSSGLYSLSSVVTVPSSSLG | |
| | TQTYICNVNHKPSNTKVDKKVEPKSCDKTHTCPPCPAP | |
| | ELLGGPSVFLFPPKPKDTLMISRTPEVTCVVVDVSHEDP | |
| | EVKFNWYVDGVEVHNAKTKPREEQYNSTYRVVSVLTV | |
| | LHQDWLNGKEYKCKVSNKALPAPIEKTISKAKGQPREP | |
| | QVYTLPPSRDELTKNQVSLTCLVKGFYPSDIAVEWESN | |
| | GQPENNYKTTPPVLDSDGSFFLYSKLTVDKSRWQQGNV | |
| | FSCSVMHEALHNHYTQKSLSLSPGK | |
| ST-23F Second | AQVLTQTPSSTSAAVGGTVTINCQASQSVVDNNWLAW | SEQ. ID. NO.: |
| Monoclonal Antibody | YQQKPGQPPKQLIYRASNLAAGVPSRFKGSGSGPQFTLT | 279 |
| (Rabbit) | ISDLECDDAATYFCGAHVSFGIFGFGGGTEVVVKGDPV | |
| Full Length Light Chain | APTVLIFPPAADQVATGTVTIVCVANKYFPDVTVTWEV | |
| | DGTTQTTGIENSKTPQNSADCTYNLSSTLTLTSTQYNSH | |
| | KEYTCKVTQGTTSVVQSFNRGDC | |
| ST-23F Second | QSVEESGGRLVTPGTPLTLTCTVSGFSLSNHGVNWVRQ | SEQ. ID. NO.: |
| Monoclonal Antibody | APGKGLEWIGIINGNGGTWYASWAKGRFTISKTSTTVD | 280 |
| (Rabbit) | LKMTSPTTEDTATYFCARGGAGAADSDIWGPGTLVTVS | |
| Full Length Heavy | LGQPKAPSVFPLAPCCGDTPSSTVTLGCLVKGYLPEPVT | |
| Chain | VTWNSGTLTNGVRTFPSVRQSSGLYSLSSVVSVTSSSQP | |
| | VTCNVAHPATNTKVDKTVAPSTCSKPTCPPPELLGGPSV | |
| | FIFPPKPKDTLMISRTPEVTCVVVDVSQDDPEVQFTWYI | |
| | NNEQVRTARPPLREQQFNSTIRVVSTLPIAHQDWLRGKE | |
| | FKCKVHNKALPAPIEKTISKARGQPLEPKVYTMGPPREE | |
| | LSSRSVSLTCMINGFYPSDISVEWEKNGKAEDNYKTTPA | |
| | VLDSDGSYFLYSKLSVPTSEWQRGDVFTCSVMHEALHN | |
| | HYTQKSISRSPGK | |
| ST-33F First | QASESIGSYLS | SEQ. ID. NO.: |
| Monoclonal Antibody | | 281 |
| (Human) | | |
| Light Chain CDR 1 | | |
| ST-33F First | YASTLAS | SEQ. ID. NO.: |
| Monoclonal Antibody | | 282 |
| (Human) | | |
| Light Chain CDR 2 | | |
| ST-33F First | AGYKNWINDEYP | SEQ. ID. NO.: |
| Monoclonal Antibody | | 283 |
| (Human) | | |
| Light Chain CDR 3 | | |
| ST-33F First | GFSLSAYDMS | SEQ. ID. NO.: |
| Monoclonal Antibody | | 284 |
| (Human) | | |
| Heavy Chain CDR 1 | | |
| ST-33F First | IIDTGGSAYYMNWAKG | SEQ. ID. NO.: |
| Monoclonal Antibody | | 285 |
| (Human) | | |
| Heavy Chain CDR 2 | | |
| ST-33F First | VPWSSDSGSYLNL | SEQ. ID. NO.: |
| Monoclonal Antibody | | 286 |
| (Human) | | |
| Heavy Chain CDR 3 | | |
| ST-33F Second | QASESIGSYLS | SEQ. ID. NO.: |
| Monoclonal Antibody | | 287 |
| (Rabbit) | | |
| Light Chain CDR 1 | | |
| ST-33F Second | YASTLAS | SEQ. ID. NO.: |
| Monoclonal Antibody | | 288 |
| (Rabbit) | | |
| Light Chain CDR 2 | | |
| ST-33F Second | AGYKNWINDEYP | SEQ. ID. NO.: |
| Monoclonal Antibody | | 289 |
| (Rabbit) | | |
| Light Chain CDR 3 | | |
| ST-33F Second | GFSLSAYDMS | SEQ. ID. NO.: |
| Monoclonal Antibody | | 290 |
| (Rabbit) | | |
| Heavy Chain CDR 1 | | |
| ST-33F Second | IIDTGGSAYYMNWAKG | SEQ. ID. NO.: |
| Monoclonal Antibody | | 291 |
| (Rabbit) | | |
| Heavy Chain CDR 2 | | |
| ST-33F Second | VPWSSDSGSYLNL | SEQ. ID. NO.: |
| Monoclonal Antibody | | 292 |
| (Rabbit) | | |
| Heavy Chain CDR 3 | | |
| ST-33F First | ALVMTQTPSPVSAAVGSTVTISCQASESIGSYLSWYQQK | SEQ. ID. NO.: |
| Monoclonal Antibody | PGQPPKLLIYYASTLASGVPSRFSGSGSGTQFTLTISGVE | 293 |
| (Human) | CDDAATYYCAGYKNWINDEYPFGGGTEVVVK | |
| Variable Light Chain | | |
| ST-33F First | QSVEESGGRLVTPGTPLTLTCTASGFSLSAYDMSWVRQ | SEQ. ID. NO.: |
| Monoclonal Antibody | APGKGLEWIGIIDTGGSAYYMNWAKGRFTISRTSTAVD | 294 |
| (Human) | LKMTSLTTEDTATYFCARVPWSSDSGSYLNLWGPGTLV | |
| Variable Heavy Chain | TVSS | |
| ST-33F Second | ALVMTQTPSPVSAAVGSTVTISCQASESIGSYLSWYQQK | SEQ. ID. NO.: |
| Monoclonal Antibody | PGQPPKLLIYYASTLASGVPSRFSGSGSGTQFTLTISGVE | 295 |
| (Rabbit) | CDDAATYYCAGYKNWINDEYPFGGGTEVVVK | |
| Variable Light Chain | | |
| ST-33F Second | QSVEESGGRLVTPGTPLTLTCTASGFSLSAYDMSWVRQ | SEQ. ID. NO.: |
| Monoclonal Antibody | APGKGLEWIGIIDTGGSAYYMNWAKGRFTISRTSTAVD | 296 |
| (Rabbit) | LKMTSLTTEDTATYFCARVPWSSDSGSYLNLWGPGTLV | |
| Variable Heavy Chain | TVSS | |
| ST-33F First | ALVMTQTPSPVSAAVGSTVTISCQASESIGSYLSWYQQK | SEQ. ID. NO.: |
| Monoclonal Antibody | PGQPPKLLIYYASTLASGVPSRFSGSGSGTQFTLTISGVE | 297 |
| (Human) | CDDAATYYCAGYKNWINDEYPFGGGTEVVVKRTVAAP | |
| Full Length Light Chain | SVFIFPPSDEQLKSGTASVVCLLNNFYPREAKVQWKVD | |
| | NALQSGNSQESVTEQDSKDSTYSLSSTLTLSKADYEKH | |
| | KVYACEVTHQGLSSPVTKSFNRGEC | |
| ST-33F First | QSVEESGGRLVTPGTPLTLTCTASGFSLSAYDMSWVRQ | SEQ. ID. NO.: |
| Monoclonal Antibody | APGKGLEWIGIIDTGGSAYYMNWAKGRFTISRTSTAVD | 298 |
| (Human) | LKMTSLTTEDTATYFCARVPWSSDSGSYLNLWGPGTLV | |
| Full Length Heavy | TVSSASTKGPSVFPLAPSSKSTSGGTAALGCLVKDYFPE | |
| Chain | PVTVSWNSGALTSGVHTFPAVLQSSGLYSLSSVVTVPSS | |
| | SLGTQTYICNVNHKPSNTKVDKKVEPKSCDKTHTCPPC | |
| | PAPELLGGPSVFLFPPKPKDTLMISRTPEVTCVVVDVSH | |
| | EDPEVKFNWYVDGVEVHNAKTKPREEQYNSTYRVVSV | |
| | LTVLHQDWLNGKEYKCKVSNKAL | |
| | PAPIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLSL | |
| | SPGK | |
| ST-33F Second | ALVMTQTPSPVSAAVGSTVTISCQASESIGSYLSWYQQK | SEQ. ID. NO.: |
| Monoclonal Antibody | PGQPPKLLIYYASTLASGVPSRFSGSGSGTQFTLTISGVE | 299 |
| (Rabbit) | CDDAATYYCAGYKNWINDEYPFGGGTEVVVKGDPVAP | |
| Full Length Light Chain | TVLIFPPAADQVATGTVTIVCVANKYFPDVTVTWEVDG | |
| | TTQTTGIENSKTPQNSADCTYNLSSTLTLTSTQYNSHKE | |
| | YTCKVTQGTTSVVQSFNRGDC | |
| ST-33F Second | QSVEESGGRLVTPGTPLTLTCTASGFSLSAYDMSWVRQ | SEQ. ID. NO.: |
| Monoclonal Antibody | APGKGLEWIGIIDTGGSAYYMNWAKGRFTISRTSTAVD | 300 |
| (Rabbit) | LKMTSLTTEDTATYFCARVPWSSDSGSYLNLWGPGTLV | |
| Full Length Heavy | TVSSGQPKAPSVFPLAPCCGDTPSSTVTLGCLVKGYLPE | |
| Chain | PVTVTWNSGTLTNGVRTFPSVRQSSGLYSLSSVVSVTSS | |
| | SQPVTCNVAHPATNTKVDKTVAPSTCSKPTCPPPELLGG | |
| | PSVFIFPPKPKDTLMISRTPEVTCVVVDVSQDDPEVQFT | |
| | WYINNEQVRTARPPLREQQFNSTIRVVSTLPIAHQDWLR | |
| | GKEFKCKVHNKALPAPIEKT | |
| | ISKARGQPLEPKVYTMGPPREELSSRSVSLTCMINGFYPS | |
| | DISVEWEKNGKAEDNYKTTPAVLDSDGSYFLYSKLSVP | |
| | TSEWQRGDVFTCSVMHEALHNHYTQKSISRSPGK | |

The present invention encompasses the antibodies (the first monoclonal and second monoclonal antibodies) defined herein as SEQ. ID. NOs.: 1-300 having the recited CDR sequences and/or variable light and variable heavy chain sequences and/or full length light and full length heavy chain sequences (otherwise known as reference antibodies (reference monoclonal antibodies)), as well as functional variants thereof. A functional variant binds to the same target antigen as the reference antibody. The functional variants may have a different affinity for the target antigen when compared to the reference antibody, but substantially the same affinity is preferred.

In one aspect, the invention relates to a monoclonal antibody (mAb), or a functional variant thereof, that specifically binds to a pneumococcal serotype (ST) capsular polysaccharide (PnPs), wherein said mAb comprises:
a) six complementarity determining regions (CDRs) selected from the group consisting of SEQ. ID. NOs.: 1-6, 21-26, 41-46, 61-66, 81-86, 101-106, 121-126, 141-146, 161-166, 181-186, 201-206, 221-226, 241-246, 261-266, 281-286, 7-12, 27-32, 47-52, 67-72, 87-92, 107-112, 127-132, 147-152, 167-172, 187-192, 207-212, 227-232, 247-252, 267-272, and 287-292;
b) a variable heavy chain and a variable light chain having an amino acid sequence selected from the group consisting of SEQ ID NOs: 13-14, 33-34, 53-54, 73-74, 93-94, 113-114, 133-134, 153-154, 173-174, 193-194, 213-214, 233-234, 253-254, 273-274, 293-294, 15-16, 35-36, 55-56, 75-76, 95-96, 115-116, 135-136, 155-156, 175-176, 195-196, 215-216, 235-236, 255-256, 275-276, and 295-296; or
c) a full length light chain and a full length heavy chain having an amino acid sequence selected from the group consisting of SEQ ID NOs: 17-18, 37-38, 57-58, 77-78, 97-98, 117-118, 137-138, 157-158, 177-178, 197-198, 217-218, 237-238, 257-258, 277-278, 297-298, 19-20, 39-40, 59-60, 79-80, 99-100, 119-120, 139-140, 159-160, 179-180, 199-200, 219-220, 239-240, 259-260, 279-280, and 299-300.

In one embodiment, the invention provides a monoclonal antibody comprising six CDRs selected from the group consisting of SEQ. ID. NOs.: 1-6, 21-26, 41-46, 61-66, 81-86, 101-106, 121-126, 141-146, 161-166, 181-186, 201-206, 221-226, 241-246, 261-266, and 281-286.

In another embodiment, the invention provides a monoclonal antibody comprising six CDRs selected from the group consisting of SEQ. ID. NOs.: 7-12, 27-32, 47-52, 67-72, 87-92, 107-112, 127-132, 147-152, 167-172, 187-192, 207-212, 227-232, 247-252, 267-272, and 287-292.

In yet another embodiment, the invention provides a monoclonal antibody comprising a variable heavy chain and a variable light chain having an amino acid sequence selected from the group consisting of SEQ ID NOs: 13-14, 33-34, 53-54, 73-74, 93-94, 113-114, 133-134, 153-154, 173-174, 193-194, 213-214, 233-234, 253-254, 273-274, and 293-294.

In a further embodiment, the invention provides a monoclonal antibody comprising a variable heavy chain and a variable light chain having an amino acid sequence selected from the group consisting of SEQ ID NOs: 15-16, 35-36, 55-56, 75-76, 95-96, 115-116, 135-136, 155-156, 175-176, 195-196, 215-216, 235-236, 255-256, 275-276, and 295-296.

In another embodiment, the invention provides a monoclonal antibody comprising a full length light chain and a full length heavy chain having an amino acid sequence selected from the group consisting of SEQ ID NOs: 17-18, 37-38, 57-58, 77-78, 97-98, 117-118, 137-138, 157-158, 177-178, 197-198, 217-218, 237-238, 257-258, 277-278, and 297-298.

In another embodiment, the invention provides a monoclonal antibody comprising a full length light chain and a full length heavy chain having an amino acid sequence selected from the group consisting of SEQ ID NOs: 19-20, 39-40, 59-60, 79-80, 99-100, 119-120, 139-140, 159-160, 179-180, 199-200, 219-220, 239-240, 259-260, 279-280, and 299-300.

In one embodiment functional variants of a reference antibody show sequence variation at one or more CDRs when compared to corresponding reference CDR sequences. Thus, a functional antibody variant may comprise a functional variant of a CDR. Where the term “functional variant” is used in the context of a CDR sequence, this means that the CDR has at most 2, preferably at most 1 amino acid difference when compared to a corresponding reference CDR sequence, and when combined with the remaining 5 CDRs (or variants thereof) enables the variant antibody to bind to the same target antigen as the reference antibody.

In one embodiment a variant antibody comprises: a light chain CDR1 having at most 2 amino acid differences when compared to a corresponding reference CDR sequence; a light chain CDR2 having at most 2 amino acid differences when compared to a corresponding reference CDR sequence; a light chain CDR3 having at most 2 amino acid differences when compared to a corresponding reference CDR sequence; a heavy chain CDR1 having at most 2 amino acid differences when compared to a corresponding reference CDR sequence; a heavy chain CDR2 having at most 2 amino acid differences when compared to a corresponding reference CDR sequence; a heavy chain CDR3 having at most 2 amino acid differences when compared to a corresponding reference CDR sequence; wherein the variant antibody binds to the same target antigen as the reference antibody.

Preferably, a variant antibody comprises: a light chain CDR 1 having at most 1 amino acid difference when compared to a corresponding reference CDR sequence; a light chain CDR2 having at most 1 amino acid difference when compared to a corresponding reference CDR sequence; a light chain CDR3 having at most 1 amino acid difference when compared to a corresponding reference CDR sequence; a heavy chain CDR1 having at most 1 amino acid difference when compared to a corresponding reference CDR sequence; a heavy chain CDR2 having at most 1 amino acid difference when compared to a corresponding reference CDR sequence; a heavy chain CDR3 having at most 1 amino acid difference when compared to a corresponding reference CDR sequence; wherein the variant antibody binds to the same target antigen as the reference antibody.

For example, a variant of the first antibody may comprise: a light chain CDR 1 having at most 2 amino acid differences when compared to SEQ ID NO: 1; a light chain CDR2 having at most 2 amino acid differences when compared to SEQ ID NO: 2; a light chain CDR3 having at most 2 amino acid differences when compared to SEQ ID NO: 3; a light chain CDR4 having at most 2 amino acid differences when compared to SEQ ID NO: 4; a light chain CDR5 having at most 2 amino acid differences when compared to SEQ ID NO: 5; a light chain CDR6 having at most 2 amino acid differences when compared to SEQ ID NO: 6; wherein the variant antibody binds to a S. pneumoniae ST-X capsular polysaccharide (CP).

For example, a variant of the first antibody may comprise: a light chain CDR 1 having at most 1 amino acid difference when compared to SEQ ID NO: 1; a light chain CDR2 having at most 1 amino acid difference when compared to SEQ ID NO: 2; a light chain CDR3 having at most 1 amino acid difference when compared to SEQ ID NO: 3; a light chain CDR4 having at most 1 amino acid difference when compared to SEQ ID NO: 4; a light chain CDR5 having at most 1 amino acid difference when compared to SEQ ID NO: 5; a light chain CDR6 having at most 1 amino acid difference when compared to SEQ ID NO: 6; wherein the variant antibody binds to a S. pneumoniae ST-X capsular polysaccharide (CP).

The foregoing can be applied analogously to variants of the other antibodies described herein (the second monoclonal antibodies), wherein the amino acid differences are defined relative to the CDR sequences thereof, and wherein the variant antibody binds to the same target antigen as said antibodies.

In one embodiment a variant antibody may have at most 5, 4 or 3 amino acid differences total in the CDRs thereof when compared to a corresponding reference antibody, with the proviso that there is at most 2 (preferably at most 1) amino acid differences per CDR. Preferably a variant antibody has at most 2 (more preferably at most 1) amino acid differences in total in the CDRs thereof when compared to a corresponding reference antibody, with the proviso that there is at most 2 amino acid differences per CDR. More preferably a variant antibody has at most 2 (more preferably at most 1) amino acid differences total in the CDRs thereof when compared to a corresponding reference antibody, with the proviso that there is at most 1 amino acid difference per CDR.

The amino acid difference may be an amino acid substitution, insertion or deletion. In one embodiment, the amino acid difference is a conservative amino acid substitution as described herein.

In one embodiment, a variant antibody has the same framework sequences as the exemplary antibodies (the first monoclonal and second monoclonal antibodies) described herein. In another embodiment the variant antibody may comprise a framework region having at most 2, preferably at most 1 amino acid difference (when compared to a corresponding reference framework sequence). Thus, each framework region may have at most 2, preferably at most 1 amino acid difference (when compared to a corresponding reference framework sequence).

In one embodiment a variant antibody may have at most 5, 4 or 3 amino acid differences total in the framework regions thereof when compared to a corresponding reference antibody, with the proviso that there is at most 2 (preferably at most 1) amino acid differences per framework region. Preferably a variant antibody has at most 2 (more preferably at most 1) amino acid differences in total in the framework regions thereof when compared to a corresponding reference antibody, with the proviso that there is at most 2 amino acid differences per framework region. More preferably a variant antibody has at most 2 (more preferably at most 1) amino acid differences total in the framework regions thereof when compared to a corresponding reference antibody, with the proviso that there is at most 1 amino acid difference per framework region.

Thus, a variant antibody may comprise a variable light chain and a variable heavy chain as described herein, wherein: the light chain has at most 14 amino acid differences (at most 2 amino acid differences in each CDR and at most 2 amino acid differences in each framework region) when compared to a light chain sequence herein; the heavy chain has at most 14 amino acid differences (at most 2 amino acid differences in each CDR and at most 2 amino acid differences in each framework region) when compared to a heavy chain sequence herein; wherein the variant antibody binds to the same target antigen as the reference antibody.

Said variant light or heavy chains may be referred to as “functional equivalents” of the reference light and heavy chains.

In one embodiment a variant antibody may comprise a variable light chain and variable heavy chain as described herein, wherein: the light chain has at most 7 amino acid differences (at most 1 amino acid differences in each CDR and at most 1 amino acid differences in each framework region) when compared to a light chain sequence herein; the heavy chain has at most 7 amino acid differences (at most 1 amino acid differences in each CDR and at most 1 amino acid differences in each framework region) when compared to a heavy chain sequence herein; wherein the variant antibody binds to the same target antigen as the reference antibody.

Suitably, each of the antibodies (first and second monoclonal antibodies) may be contacted with the sample in a discrete compartment. The discrete compartment may be a multi-well plate. The discrete compartment may be a 96-well plate. The multi-well plate may be covered with a plate sealer.

The capsular polysaccharides (the PnPs) detectable by a method of the present invention are serotype (ST)-specific. Thus, for example, where a serotype 1 (ST-1) capsular polysaccharide is detected in a sample, this indicates the presence of S. pneumoniae “1”. The capsular polysaccharide may be independent of a bacterium (e.g. no longer integral to the bacterial membrane), yet may provide an indication of the presence or absence of said bacterium within a sample (e.g. presence or absence of an infection with said bacterium). This is highly advantageous, as direct detection of the bacterium (e.g. intact bacterium) is not required, and a free capsular polysaccharide may be used as a proxy/indicator of the presence of the bacterium.

The present invention allows for the detection of the specific serotypes in the sample, or serotyping of a S. pneumoniae (e.g. isolated S. pneumoniae). Advantageously, this allows for diagnosis of a subject with an infection of specific serotypes of S. pneumoniae, and thus administration of a therapy suitable for treating said serotype. The method of the invention may be used for diagnosing infection of a subject with a S. pneumoniae (e.g. a specific serotype of S. pneumoniae).

Thus, a method of the present invention enables rapid determination of the S. pneumoniae serotype present in a sample. Similarly, the invention provides a rapid method for the confirmation that all of said serotypes are absent from the sample by way of a multiplex method. A multiplex method means that a plurality of assays are performed, preferably under the same assay conditions and/or substantially at the same time. Alternatively, the assays may be performed at separate times.

Furthermore, the methods do not require culturing of bacteria isolated from a subject, and can be performed on samples (e.g. crude samples) directly isolated from a subject. The existing “gold standard” assay for serotyping a broad spectrum of serotypes is the “Quellung reaction”. While this method is capable of identifying numerous pneumococcal serotypes, it requires the use of many specific pneumococcal antisera (e.g. polyclonal antibodies) and is costly and laborious. A significant drawback of this method of typing is that it requires the recovery of a viable pneumococcal culture and thus precludes any case where an isolate is not obtainable—for example, when antimicrobial treatment has been administered prior to specimen collection, or in cases of non-invasive disease. The BinaxNOW™ pneumococcal test (Alere) can detect pneumococcal cell wall C polysaccharide (CWP) in samples (via a CWP polyclonal antibody). However, this test is not capable of reporting any serotype-specific information. Molecular techniques (e.g. PCR) for serotyping suffer from requiring the conditions for each assay to be individually optimized, and from requiring a multitude of complex component parts.

A wide spectrum of S. pneumoniae serotype-specific capsular polysaccharides (PnPs) can be detected by methods of the present invention due to the provision of an array of monoclonal antibodies which bind said serotype-specific capsular polysaccharides (PnPs). The inventors have demonstrated that the presence or absence of 15 or more S. pneumoniae serotype-specific capsular polysaccharides (PnPs) can be detected by methods of the present invention.

In one embodiment, a method of the invention may further comprise administering to said subject a therapy suitable for treating an infection with a S. pneumoniae serotype comprising said capsular polysaccharide.

Suitable treatments include one or more of an antibiotic selected from penicillin, cefotaxime, erythromycin and co-trimoxazole.

In one embodiment, a method of the invention further comprises contacting the sample with one or more first and second monoclonal antibodies (mAbs), to form one or more first mAb-antigen-second mAb complexes, wherein the one or more first and second monoclonal antibodies (mAbs) bind to one or more S. pneumoniae serotype-specific capsular polysaccharides.

Detection of a serotype-specific capsular polysaccharide can be used to indicate the presence of the corresponding bacterium serotype in the sample, or in the subject from which the sample is isolated. For example, a free capsular polysaccharide (released from a bacterium) may be detected in the sample, indicating the presence of the bacterium serotype in the subject.

In one embodiment, the method may be used to diagnose a subject with an infection with a S. pneumoniae serotype, wherein the presence of the first mAb-antigen-second mAb complex is indicative of the presence of an infection with a serotype comprising capsular polysaccharide, and wherein the absence of the first mAb-antigen-second mAb complex is indicative of the absence of an infection with a serotype comprising said capsular polysaccharide.

The term “diagnosis” as used herein encompasses identification, confirmation and/or characterization of S. pneumoniae serotype infection. Methods of diagnosis according to the invention are useful to confirm the existence of an infection. Methods of diagnosis according to the invention are also useful in methods of assessment of clinical screening, prognosis, choice of therapy, evaluation of therapeutic benefit, i.e. for drug screening and drug development. Efficient diagnosis allows rapid identification of the most appropriate treatment (thus lessening unnecessary exposure to harmful drug side effects), and reducing relapse rates.

In another aspect, there is provided a method for determining prognosis of an infection with a S. pneumoniae serotype in a subject, comprising detecting the presence or absence of a serotype-specific capsular polysaccharide through a method of the invention. In such aspects, the presence of the first mAb-antigen-second mAb complex is indicative of (e.g. correlates with) a poor prognosis for an infection with a S. pneumoniae serotype comprising said capsular polysaccharide, and the absence of the first mAb-antigen-second mAb complex is indicative of (e.g. correlates with) a good prognosis for in infection with a S. pneumoniae serotype comprising said capsular polysaccharide.

In one embodiment, a sample may be one or more selected from saliva, blood (e.g. whole blood, blood serum or blood plasma), mucous, sputum, cerebrospinal fluid, synovial fluid, a lesion, bodily fluid isolated from a lesion, eye fluid, lymphatic fluid, seminal fluid, and/or sebaceous fluid.

In one embodiment, the sample is obtained from surgical or other material equipment. In one embodiment, the sample is an environmental sample (e.g. water, soil and/or sediment).

In a preferable embodiment, the sample is urine. Suitably, said urine sample may be isolated from a subject suspected of having an infection with a S. pneumoniae serotype. In some embodiments, the sample is isolated from a subject diagnosed as having an S. pneumoniae infection.

A key advantage to using a urine sample in the methods of the present invention is that this sample is readily obtainable from a subject having or suspected of having an infection with a S. pneumoniae serotype and is obtained without the need for invasive techniques.

In one embodiment, a sample may be processed to isolate a S. pneumoniae serotype from a sample prior to detecting the presence or absence of S. pneumoniae serotype capsular polysaccharide. In one embodiment, the S. pneumoniae is cultured from a bacterium isolated from a subject and the resulting culture is applied to an assay in methods of the invention.

The terms “subject”, “individual” and “patient” are used interchangeably herein to refer to a mammalian subject. In one embodiment the “subject” is a human, a companion animal (e.g. a pet such as dogs, cats, and rabbits), livestock (e.g. pigs, sheep, cattle, and goats), and horses. In a preferable embodiment, the subject is a human. In methods of the invention, the subject may not have been previously diagnosed as having an S. pneumoniae infection. Alternatively, the subject may have been previously diagnosed as having an S. pneumoniae infection. The subject may also be one who exhibits disease risk factors, or one who is asymptomatic for an S. pneumoniae infection. The subject may also be one who is suffering from or is at risk of developing an S. pneumoniae infection. Thus, in one embodiment, a method of the invention may be used to confirm the presence of an S. pneumoniae (serotype) infection in a subject. For example, the subject may previously have been diagnosed with S. pneumoniae (serotype) infection by alternative means. In one embodiment, the subject has been previously administered an S. pneumoniae (serotype) therapy.

“Specific binding” or “specifically binds to” or is “specific for” a particular antigen (polysaccharide antigen), target, or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

A “mAb-antigen complex” means a complex (e.g. macromolecular complex) comprising a capsular polysaccharide antigen which has become bound to a mAb (e.g. a mAb with binding affinity for said capsular polysaccharide antigen). The term “mAb-antigen complex” is synonymous with the terms “bound capsular polysaccharide-mAb complex” and “mAb bound to a capsular polysaccharide”. For example, a “mAb-antigen complex” means a complex (e.g. macromolecular complex) comprising a capsular polysaccharide antigen which has become bound to a first mAb (e.g. a mAb with binding affinity for said capsular polysaccharide antigen).

A “mAb-antigen-secondary antibody complex” means a complex comprising a capsular polysaccharide antigen which has become bound to a mAb (e.g. a mAb with binding affinity for said capsular polysaccharide antigen), wherein said complex has further become bound by a secondary antibody which binds said capsular polysaccharide and/or capsular polysaccharide-mAb complex. For example, a “mAb-antigen-secondary antibody complex” means a complex comprising a capsular polysaccharide antigen which has become bound to a first mAb (e.g. a mAb with binding affinity for said capsular polysaccharide antigen), wherein said complex has further become bound to a second mAb which binds said capsular polysaccharide and/or capsular polysaccharide-mAb complex.

In an embodiment, the first mAb of the present invention is a human and/or mouse mAb. In an embodiment, the second mAb of the present invention is a rabbit mAb.

In one embodiment, the first monoclonal antibody (mAb) may be immobilized on a surface. Preferably, the first mAb may be immobilized on (e.g. absorbed to) the surface of a bead. In one embodiment, said bead may be constructed with/from a carboxylated polystyrene material. Preferably, said bead may be a carboxylated polystyrene microsphere.

In one embodiment, the first monoclonal antibody (mAb) may be immobilized on the surface of a discrete compartment. Said discrete compartment may be a test tube (e.g. a glass test tube) or an “eppendorf” tube or a plate.

In one embodiment, a method of the invention is a multiplex method wherein said contacting step is performed simultaneously and preferably under the same conditions.

In one embodiment, each first and second mAb may be present within a discrete compartment, and the urine sample may be contacted with the first and second mAb within said discrete compartment. Thus, each first and second mAb may be contacted with the urine to provide a plurality of discrete assays.

Conditions (e.g. assay conditions) during the method are preferably kept consistent, preferably without the need for optimization of conditions for individual assays. For example, the volume of sample applied to each assay is preferably the same, as are the time (e.g. incubation) and temperature conditions, etc.

In a preferable embodiment, a sample is incubated with the first mAb to allow capsular polysaccharide in the sample to contact with the first mAb within the assay forming the first mAb-antigen complex. Said incubation may be for any time between about 30 minutes, and about 72 hours (e.g. about, 30 minutes, about 1 hour, about 2 hours, about 3 hours, about 4 hours, about 5 hours, about 6 hours, about 12 hours, about 18 hours, about 24 hours, about 36 hours, about 48 hours, about 60 hours, or about 72 hours). Suitably, said incubation is for about 30 minutes, about 1 hour, about 2 hours, or about 3 hours. Preferably, said incubation is for about 2 hours.

In another embodiment, a sample is incubated with the first mAb to allow capsular polysaccharide in the sample to contact with the first mAb within the assay forming the first mAb-antigen complex. Said incubation may be for any time between 30 minutes and 72 hours (e.g. 30 minutes, 1 hour, 2 hours, 3 hours, 4 hours, 5 hours, 6 hours, 12 hours, 18 hours, 24 hours, 36 hours, 48 hours, 60 hours, or 72 hours). Suitably, said incubation is for 30 minutes, 1 hour, 2 hours, or 3 hours. Preferably, said incubation is for 2 hours.

In a preferable embodiment, next the sample is washed.

In a preferable embodiment, next the sample is incubated with the second mAb to allow capsular polysaccharide in the sample to contact with the second mAb within the assay forming the first mAb-antigen-second mAb complex. Said incubation may be for any time between about 30 minutes and about 48 hours (e.g. about 30 minutes, about 1 hour, about 3 hours, about 6 hours, about 12 hours, or about 24 hours). Suitably, said incubation is for about 30 minutes. Suitably, said incubation is for about 45 minutes. Suitably, said incubation is for about 1 hour.

In another embodiment, the sample is incubated with the second mAb to allow capsular polysaccharide in the sample to contact with the second mAb within the assay forming the first mAb-antigen-second mAb complex. Said incubation may be for any time between 30 minutes and 48 hours (e.g. 30 minutes, 1 hour, 3 hours, 6 hours, 12 hours, or 24 hours). Suitably, said incubation is for 30 minutes. Suitably, said incubation is for 45 minutes. Suitably, said incubation is for 1 hour.

In a preferable embodiment, next the sample is washed.

In a preferable embodiment, next the sample incubated with the third antibody to allow the third antibody to contact with the second mAb within the assay. Said incubation may be for any time between about 30 minutes and about 48 hours (e.g. about 30 minutes, about 1 hour, about 3 hours, about 6 hours, about 12 hours, or about 24 hours). Suitably, said incubation is for about 30 minutes. Suitably, said incubation is for about 45 minutes. Suitably, said incubation is for about 1 hour.

In another embodiment, the sample incubated with the third antibody to allow the third antibody to contact with the second mAb within the assay. Said incubation may be for any time between 30 minutes and 48 hours (e.g. 30 minutes, 1 hour, 3 hours, 6 hours, 12 hours, or 24 hours). Suitably, said incubation is for 30 minutes. Suitably, said incubation is for 45 minutes. Suitably, said incubation is for 1 hour.

In one embodiment, next the sample is washed.

In one embodiment, next the sample is read in a reader (for example, a Luminex Reader™).

In one embodiment, the method of the invention comprises performing each assay in a discrete compartment (e.g. of an immunoassay apparatus), preferably wherein said sample is applied to wells.

Any means (e.g. apparatus) in which an assay may be performed may constitute a “compartment” (e.g. of an immunoassay apparatus) as described herein. In one embodiment, a “compartment” is a discrete well (e.g. of a multi-well plate). In another embodiment, a “compartment” is a tube (e.g. a test tube or “eppendorf” tube).

Methods of the invention have high levels of sensitivity and specificity.

In one embodiment, a capsular polysaccharide is detected at a concentration of ≥about 0.003 ng/mL, 0.01 ng/ml, 0.1 ng/mL, 0.3 ng/mL, or 1 ng/ml. In one embodiment, a capsular polysaccharide is detected at a concentration of greater than or equal to (≥) about 0.008 ng/mL.

Preferably, a (particular polysaccharide(s)) is detected at a concentration of (e.g. a concentration as low as) ≥about 0.008 ng/ml. Preferably, a (different particular polysaccharide(s)) is detected at a concentration of (e.g. a concentration as low as) ≥about 0.03 ng/mL.

In one embodiment, a capsular polysaccharide is detected with a specificity of at least 85% (e.g. at least 90%, 95% or 100%). In another embodiment, a capsular polysaccharide is detected with a specificity of about 98% (e.g. 98.4%, 95%, 99.7%). Specificity is determined by assessing the ability to measure and report the presence or absence of specific capsular polysaccharides in the sample. Specificity may be determined as set out in Example A below.

Example A: A negative urine is spiked with 14 of the 15 serotype specific capsular polysaccharide and the sample tested with Luminex x-MAP™ technology. The assay ability to detect the absence of missing polysaccharide establish the specificity for the target capsular polysaccharide.

In one embodiment, the method of the invention is performed with Luminex x-MAP™ technology.

In another embodiment, the method of the invention comprises applying the urine sample to a control assay comprising no mAb. Alternatively, said control assay may comprise a mAb, but no urine sample is applied to it.

In another aspect, there is provided a kit for detecting the presence or absence of a S. pneumoniae capsular polysaccharide(s) (PnPs) in a sample, wherein said kit comprises:
a) a first monoclonal antibody (mAb) that binds a pneumococcal serotype (ST) capsular polysaccharide (PnPs);
b) a second monoclonal (mAb) that binds the pneumococcal ST PnPs;
c) a third antibody; and
d) instructions to use said kit.

In another aspect, there is provided a kit for detecting the presence or absence of a S. pneumoniae capsular polysaccharide(s) (PnPs) in a sample, wherein said kit comprises:
a) a first monoclonal antibody (mAb) that is capable of binding a PnPs in a sample to form a first mAb-antigen complex;
b) a second mAb that is capable of binding a PnPs in a sample to form a first mAb-antigen-second mAb complex;
c) a third antibody that is capable of binding to the second mAb; and
d) instructions to use said kit to detect the presence or absence of said first mAb-antigen-second mAb complex and detect the presence or absence of PnPs.

In another aspect, the kit further comprises references standards and controls. In another aspect, the kit further comprises plates and optionally plate sealers. In another aspect, the kit further comprises buffers.

The term “reference standard(s)” means a preparation of individual pneumococcal serotypes, for example serotypes 1, 3, 4, 5, 6A, 6B, 7F, 9V, 14, 18C, 19A, 19F, 22F, 23F, and 33F capsule polysaccharides, in solution. The reference standard has known concentrations of the serotypes. The reference standard is included in the SSUAD assay to deduce the pneumococcal serotype specific capsular polysaccharide concentration in an unknown specimen (a sample).

The term “control(s)” means a preparation of specific concentration(s) of individual pneumococcal serotypes, for example serotypes 1, 3, 4, 5, 6A, 6B, 7F, 9V, 14, 18C, 19A, 19F, 22F, 23F, and 33F capsule polysaccharides, in negative urine.

“Plate(s) include a 96 well, black, optical-bottom plate(s) with polymer base, non-treated, 0.4 mL microwell plate(s) (Thermo Scientific™ Nunc™, Cat. #265301 or equivalent) used in the SSUAD assay.

“Buffer(s)” include: an “Activation Buffer” which contains 100 mM MES (pH 6.0±0.05) in sterile distilled water (useful for Luminex™ bead conjugation to a first mAb; a “Coupling Wash Buffer” which contains a phosphate buffer salt solution with tween (PBST), 1% BSA, and 0.05% Sodium Azide (pH 7.4±0.05), useful to store first mAb conjugated Luminex™ beads; and an “Assay Buffer” which contains a phosphate buffer salt solution with tween (PBST), 0.05% casein, and 0.05% Sodium Azide, useful for sample and reagent (antibody) dilutions in the SSUAD assay.

A “plate sealer” may be an aluminum foil sealing film which is a 38 μm, thick sealing film, useful with 96 well plates.

In an alternate embodiment, the immunoassay apparatus further comprises a secondary antibody. Said secondary antibody is suitably a detection antibody capable of binding to a capsular polysaccharide and/or capsular polysaccharide-mAb complex described herein. In an embodiment, said secondary antibody is conjugated to a detection means, preferably a fluorescent dye. Such secondary antibody is preferably used for detecting for the presence or absence of capsular polysaccharide and/or capsular polysaccharide-mAb complex within one or more assays in methods of the present invention.

An immunoassay apparatus may suitably be constructed with Luminex x-MAP Technology™. Luminex x-MAP technology involves the use of magnetic, spectrally distinct carboxylated polystyrene microspheres, or “beads” that can be coated with a broad range of molecules, including nucleic acids and proteins, and used in various assay formats, such as PCR-based assays and immunoassays. Coating such beads with antibodies specific to pneumococcal serotypes allows for the simultaneous detection of different pneumococcal serotype antigens in a single sample, thus reducing the amount of sample required. To date, Luminex™ microsphere technologies have been used to aid in the detection of pneumococcal serotypes using antibody detection of polysaccharide, competitive inhibition assays, PCR-based multiplex assays, and serological assays to detect antibody responses to pneumococcal serotypes.

In an embodiment, the monoclonal antibody is immobilized (e.g. adsorbed) on the surface of a bead such as a carboxylated polystyrene microsphere. In an embodiment a bead is fluorescent.

In another embodiment, a mAb of the invention is used as a reagent.

In another embodiment, a mAb of the invention is used as a reagent in commercial and non-commercial assays.

In another embodiment, the commercial and non-commercial assays include, but are not limited to: release assays for stability (CRM Capture ELISA Assay); release assays for potency (Total Polysaccharide ELISA Assay); PCV ID Assays; % Adsorption Assays; characterization assays (Free Polysaccharide ELISA Assay); and polysaccharide yield assays.

In another embodiment, the invention further relates to an assay for detecting multiple ST-specific PnPs in a sample from a subject wherein Luminex Laboratory MultiAnalyte Profiling Technology (Luminex Corp., Austin, TX) is used in conjunction with a LUMINEX desktop analyzer to simultaneously measure multiple ST-specific PnPs in a single sample. In one embodiment, a first and a second mAb corresponding to more than one pneumococcal serotype (ST) capsular polysaccharide (PnPs) is present within a discrete compartment, and a patient sample is contacted with the first and second mAb within said discrete compartment. In another embodiment of the invention, a plurality of monoclonal antibodies (mAb), or functional variants thereof, that bind to different pneumococcal (ST) capsular polysaccharide (PnPs) in the sample are coupled to a plurality of distinct fluorescent Luminex microspheres. The ST-specific PnPs in the sample are each associated with specific Luminex microspheres that are identified by their distinct red and infrared fluorescent dye spectral properties on the LUMINEX analyzer (Fulton et al., Clin. Chem. 43 (9): 1749-56 (1997)). The methods of the present invention accurately detects multiple ST-specific PnPs simultaneously in a sample from a patient, e.g., a urine sample.

Other definitions or terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Where a range of values is provided, it is understood that each intervening value to the tenth of the unit of the lower limit unless the context clearly dictates otherwise between the upper and lower limits of that range is also specifically disclosed. Each smaller range between any stated value or intervening value in a stated range and any other stated or intervening value in that stated range is encompassed within the disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include the plural referents unless the context clearly dictates otherwise. Thus, for example, reference to “a monoclonal antibody” includes a plurality of such monoclonal antibodies and reference to “the capsular polysaccharide” includes reference to one or more capsular polysaccharides and equivalents thereof known to those skilled in the art, and so forth.

The publications discussed herein are provided solely for their disclosure prior to the filing date of the present application. Nothing herein is to be construed as an admission that such publications constitute prior art to the claims appended hereto.

### Example 1

### Methods of Making the Antibodies of the Invention

The heavy and light chain sequence of the target mAb was cloned into transfection-grade plasmid maxi-prep for mammalian expression. ExpiCHO-S cells were grown in serum-free ExpiCHO™ Expression Medium (Thermo Fisher Scientific). The cells were maintained in Erlenmeyer Flasks (Corning Inc.) at 37° C. with 8% CO₂ on an orbital shaker. One day before transfection, the cells were seeded at an appropriate density in Corning Erlenmeyer Flasks. On the day of transfection, DNA and transfection reagent were mixed at an optimal ratio and then added into the flask with cells ready for transfection.

The recombinant plasmids encoding heavy and light chain of target mAb were transiently transfected into suspension ExpiCHO-S cell cultures. Enhancer and feed were added on day 1 and day 5 post-transfection. The cell culture supernatant collected on day 14 post-transfection or when the cell viability was less than 50% was used for purification. Cell culture broth was centrifuged followed by filtration. Filtered supernatant of mAb was loaded onto the MabSelect SuRe™ LX 30 ml (GE, Cat. No. 17-5474-02) at 6.0 ml/min. After washing and elution with appropriate buffers, the eluted fractions were pooled and buffer exchanged to 3% Sucrose, 50 mM Histidine, 50 mM Arginine, pH 6.0. To determine the molecular weight and purity, the purified antibody was subsequently analyzed by SDS-PAGE, Western blot and SEC-HPLC using standard protocols.

### Example 2

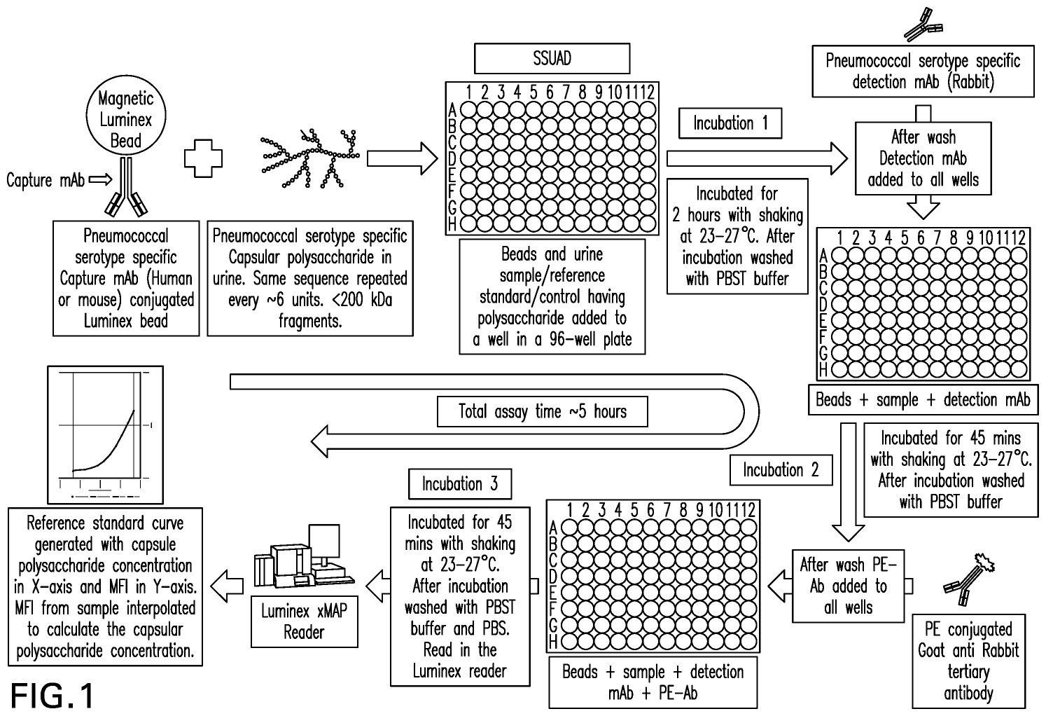

### General SSUAD Assay Protocol (See FIG. 1)

1. The SSUAD assay was performed in a 96-well plate format at 23° C.±2° C., plates were agitated on a plate shaker as indicated.
2. Human or murine ST-specific primary capture mAbs (first mAbs) were coupled to 15 different Luminex beads, using manufacturer-recommended diimide conjugation chemistry.
3. Each 96-well assay plate included standards and controls (11-point diluted reference standards, high, medium, low and negative quality controls) along with urine samples in duplicates.
4. All 15 different primary capture mAbs (first mAbs) conjugated Luminex beads were added to each well, along with sample, references and controls, and incubated at 23° C.±2° C. with agitation for 2 hours±10 mins.
5. Plates were washed 3 times with phosphate buffered saline with Tween 20 (PBST).
6. After the wash, a secondary ST-specific detection mAb (second mAb) cocktail (15-plex) was added to each well and incubated at 23° C.±2° C. with agitation for 45±5 mins.
7. Plates were washed 3 times with PBST.
8. After the wash, tertiary PE conjugated anti-rabbit antibody (the third antibody) was added to each well and incubated at 23° C.±2° C. with agitation for 45±5 mins.
9. Plates were washed 3 times with PBST and resuspended in Dulbecco's phosphate buffered saline (DPBS).
10. The assay plates were analyzed using a Luminex reader (for example a BioPlex 200 reader).

### Example 3

### SSUAD Assay Development and Optimization

### Conjugation of Monoclonal Antibodies to Luminex Beads and Overview of Assay

Pairs of monoclonal antibodies (mAbs) recognizing each of the Vaxneuvance™ serotypes were used as primary capture (the first monoclonal antibodies) and secondary reagents (the second monoclonal antibodies). Human or murine ST-specific primary capture mAbs were coupled to 15 different Luminex™ beads, using manufacturer-recommended diimide conjugation chemistry. The PnPs in the sample were captured in a sandwich binding format by the ST-specific primary and secondary (rabbit) mAbs. Tertiary goat anti-rabbit reporter antibodies coupled to phycoerythrin (PE) were used, which bind to the secondary mAbs to allow detection and quantitation of the captured analyte using the Luminex microfluidics system™ (FIG. 1).

### Incubation Time and Conditions

Incubation time, reagent concentration, and reaction conditions required for each step were optimized in singleplex format before adaptation to the multiplex format. To optimize primary (the first monoclonal antibody), secondary (the second monoclonal antibody), and tertiary (the third antibody) antibody concentrations for the SSUAD assay, 15 PnPs were tested separately at three concentrations against their respective first mAbs and conjugated beads. Pneumococcal ST-specific first mAbs were conjugated to Luminex beads at three concentrations and tested against three dilutions of secondary and tertiary antibodies.

### Adapting to a Multiplex Assay

The Luminex xMAP™ (Luminex Corp., Austin, TX) bead technology allows simultaneous detection of multiple STs of PnPs in a single well, reducing sample volume and assay time. The Luminex™ reader uses a flow cell to align the beads so that each can be individually interrogated for its spectral signature and PE-dependent median fluorescence intensity (MFI). The concentration of PnPs in the sample is determined by interpolating MFI from test samples using a serially diluted PnPs reference curve (FIG. 1).

Optimal primary and secondary mAb concentrations determined from singleplex experiments were tested in the multiplex format and adjustments were made for some STs. Design-Expert (Stat-Ease, MN) was used to optimize incubation times, assay consistency, and throughput in the multiplex format. Specificity of the PnPs mAbs used in the multiplex format was confirmed by comparing urine samples spiked with only one of each of the Vaxneuvance™ serotypes and samples containing the other 14 of 15 Vaxneuvance™ serotypes.

### Reference Standard and Controls

Commercially available PnPs-negative urine samples (Biological Specialty Corp., PA) treated with 0.5 M PIPES (Sigma, USA) to a final concentration of 25 mM were used as “no spike” (NS) negative controls, while standard curve and quality controls (QCs) were prepared by spiking NS urine with specific concentrations of full-length vaccine-grade PnPs (Merck Sharp & Dohme Corp., Kenilworth, NJ, USA).

To allow quantification of PnPs in urine samples, 11 reference standards containing all 15 of the Vaxneuvance™ serotypes were prepared in 2-fold serial dilutions, with in-well starting concentrations ranging from 0.5-20 ng/ml, depending on limits of detection. QC samples used in each run contained high, medium, low, and no PnPs concentrations in the dynamic range of the standard curve for each ST.

### Optimized Assay Procedure

The optimized SSUAD assay was conducted at 23° C.±2° C. in 96-well plates agitated on a plate shaker (FIG. 1). Primary incubation of reference standard, QCs, and test samples with capture mAbs conjugated to Luminex™ beads lasted 2 h±10 min. Subsequent incubations with secondary and tertiary antibodies were for 45 min±5 min each. Between each antibody incubation step, wells were washed with phosphate buffered saline with Tween 20 (PBST). Following the incubation of the tertiary antibodies (the third antibody), Luminex™ beads were resuspended in Dulbecco's phosphate buffered saline (DPBS) and analyzed using a Luminex™ 200 reader (BioPlex 200 reader).

### Assay Qualification

The purpose of qualification experiments was to assess accuracy, precision and ruggedness, specificity, selectivity and dilutional linearity of the SSUAD assay.

### Accuracy

Individual aliquots of eight different NS urine samples were spiked with 11 concentrations of the 15-PnPs reference standard (two-fold difference between spikes) with corresponding NS control. Relative accuracy at a particular spike concentration was considered acceptable if the average recovery across the eight test samples was between 80% and 125% of the known spike concentration.

PnPs recovery for each spiked test sample was assessed using the equation:

$$\% Relative accuracy = 100 \% \times \frac{Obseerved conc}{Expected conc} ,$$

where the observed concentration was the PnPs concentration obtained by interpolating samples against an 11-point reference standard curve tested on the same plate, and the expected concentration was the concentration spiked into the sample. Observed concentrations were corrected for matrix effects using the median response from NS samples when measurable.

### Precision and Ruggedness

Sixteen individual NS samples were spiked with one of 16 concentrations of a reference standard containing either full-length or fragmented PnPs of the 15 Vaxneuvance™ serotypes. The concentration range of spiked PnPs spanned the range of the reference standard included in each plate. NS samples were included within each run to assess background matrix effects. Four different Luminex™ instruments were used to read assay plates to assess assay ruggedness. The target for acceptable precision for each Vaxneuvance serotype was a percent coefficient of variation (% CV)<25% across all replicates. Differences between results across ruggedness criteria (analyst and Luminex™ instrument) were considered acceptable if the % CV was <30%.

Intra-assay precision was assessed on the data from the PnPs spiked samples that were tested two times within each assay run. Total or intermediate assay precision was assessed for each individual test sample (NS, and samples spiked with full length or fragmented PnPs) using all determinable concentrations. Geometric mean concentration (GMC) and % CV were calculated separately for each test sample by variance component analysis using the Mixed Procedure in SAS®. The mixed model contained random terms for Analyst and Run within Analyst. Variance component analysis was also used to assess total assay precision across the set of test samples whose GMC was within the determined LOQs. The mixed model to assess total assay precision across test samples contained a fixed term for Sample and random terms for Analyst, Run within Analyst, and their interactions with Sample. Total or intermediate precision was calculated as 100%×\sqrt{e^{V}−1} where V denotes the sum of the variance component estimates. Estimates of the differences among the levels within each ruggedness factor were the differences between the least squares mean obtained by fitting a model containing fixed terms for Analyst and Luminex Instrument to the natural log-transformed PnPs concentrations.

### Sensitivity, Limit of Detection and Limit of Quantitation

The limit of detection (LOD) of the SSUAD assay was defined as the lowest concentration of PnPs that can be detected with a signal that is significantly higher than that of the background in the absence of PnPs. The LODs were determined by spiking the PnPs reference standard at 11 concentrations (two-fold difference between spikes) into aliquots of eight different NS urine samples. The limits of quantitation (LOQ) were the lowest and highest concentrations of PnPs detected with relative accuracy within 80-125% of the known spike concentration and with total assay variability <25%.

The LOD was the minimum concentration of PnPs having, 1) a geometric mean MFI ratio ≥1.20 (MFI of spiked sample/MFI of corresponding NS sample); and 2) a lower boundary of the 95% prediction interval of the geometric mean MFI ratio that exceeds 1.0, as determined using a t-distribution. The LOQs should also be within the concentrations of the second and tenth points of the standard curve, as determined by assessing the precision of test samples and the relative accuracy of the assay.

### Specificity

Analytical specificity of the SSUAD assay was determined by assessing its ability to measure and report the presence of specific Vaxneuvance™ serotype PnPs in the sample. In each run, 15 NS samples were spiked with 14 of the 15 Vaxneuvance™ serotype PnPs, with each sample missing a different ST. The concentration of PnPs in the spiked samples of each ST corresponded to the fourth highest concentration of the standard curve. This concentration of spiked PnPs would be at a quantifiable level to detect and estimate cross-reactivity among STs. A NS negative control and an all 15 PnPs-spiked pooled urine as positive control were also included.

The GMCs were reported for each of the Vaxneuvance™ serotype spiked in each urine sample.

### Selectivity

A panel of eight different NS urine samples were diluted 2-fold with increasing amounts of assay buffer (0.05% casein in PBST) creating a series of eight dilutions ranging from neat to 1:128. Selectivity of the SSUAD assay was evaluated by measuring a known concentration of PnPs reference standards spiked into each urine sample. The final in-well concentration of PnPs corresponded to the fifth highest concentration of the standard curve, chosen to allow for quantitation at higher levels of response if the urine matrix depressed the measured concentration.

Mean percentage recovery and 90% confidence intervals were calculated for each level of diluted urine for each Vaxneuvance™ serotype.

### Dilutional Linearity

The dilutional linearity of the SSUAD assay was assessed across a range of dilutions by regressing the log₁₀-transformed dilution-corrected PnPs concentrations against the log₁₀-transformed dilutions. The PnPs reference standard was spiked into eight different NS samples then serially diluted four-fold in assay buffer for a four-point dilution series (neat, 1:4, 1:16, and 1:64). Each spiked sample contained a final concentration of spiked PnPs corresponding to the third highest concentration of the standard curve. This concentration was chosen to ensure that the neat sample would be quantifiable and permit at least four of the subsequent dilutions of the sample to be quantifiable. The predetermined acceptance criterion for dilutional linearity was that the dilution-bias per 10-fold dilution should be less than two-fold.

Dilutional linearity was assessed by regressing the log 10-transformed, dilution-corrected PnPs concentrations against the log 10-transformed dilutions. Linearity was estimated using a mixed model that included fixed terms for samples and the average dilution effect (slope), along with a random term representing the variability in the dilution effect across the individual test samples. Dilution bias per 10-fold dilution was calculated as:

$$Dilution Bias per 10 / fold dilution = 100 \% \times ( {10}^{b} - 1 )$$

where “b” represents the estimate of the average dilution effect (slope) from the mixed model.

### Clinical Validation

### Conditions Unique to Validation Experiments

Validation experiments were conducted to ensure that the performance characteristics were consistent with the acceptance criteria established during assay qualification. Clinical urine samples from 25 patients with CAP confirmed by positive culture from blood, pleural fluid, or other sterile sites were acquired from the competence network for community-acquired pneumonia study group (CAPNETZ), an ongoing prospective survey of CAP in Germany (Pletz M. W. et al., The burden of pneumococcal pneumonia-experience of the German competence network CAPNETZ. Pneumologie. 2012; 66:470-475 and Bracken D. C. W. et al., Shift in bacterial etiology from the CAPNETZ cohort in patients with community-acquired pneumonia: data over more than a decade. Infection. 2021; 49:533-537.). Ten of the 25 CAPNETZ samples were divided into two aliquots, while one other patient sample was divided into three aliquots so that, along with the remaining 14 samples, the study panel included 37 individual clinical CAP samples.

Clinical urine samples from 16 patients with non-bacteremic pneumococcal pneumonia (NBPP) and confirmed vaccine ST positivity collected in the CORE PNEUMO US surveillance study (Self W. H. et al., Incidence of All-Cause and Pneumococcal Pneumonia in Tennessee and Georgia, USA, 2018-2020: Results from the PNEUMO Study. 31st European Congress of Clinical Microbiology and Infectious Diseases (ECCMID) 2021, Online.) were also included in the panel of clinical samples during the validation experiments. Three of the 16 NBPP patient samples were divided into three aliquots, two other samples were divided into two aliquots, and one sample was divided into four aliquots so that, along with the remaining 10, the study panel included 27 individual clinical NBPP samples during testing.

For precision and ruggedness, the 37 CAPNETZ samples and the 27 CORE samples were tested four times each at a dilution factor determined during pre-screening before assay validation. Experiments were performed eight times by two analysts, four runs each (one plate per run), on separate days, with ≥1 week between the first two and last two runs. Each plate contained 32 samples tested in duplicate, with each sample randomly arranged in four of the eight runs.

The target for acceptable precision was % CV<25% for each ST. Differences across ruggedness criteria (analyst, bead lot, or Luminex™ instrument) were considered acceptable if <30%. A ‘positive’ result in the SSUAD assay was defined as a measured concentration ≥2-fold higher than the lower limit of quantitation (LLOQ). Quantitative results for each sample tested with the SSUAD assay were compared with the clinical culture results obtained previously.

### Example 4

### Assay Development and Optimization

The SSUAD assay was developed and optimized to detect and quantify the Vaxneuvance™ serotypes in 15-plex format (FIG. 1). During the multiplex optimization process, STs 1, 7F, 18C, 19F, and 23F required additional adjustments to their capture mAb and secondary mAb reaction concentrations. The final optimized SSUAD assay conditions are provided in Table 1.

TABLE 1
Multiplex SSUAD Optimized Assay Conditions

| | | sAB Dilution | | | | | | |
|---|---|---|---|---|---|---|---|---|
| | Capture | | Concentration | In-well concentration (ng/mL) | | | | |
| | mAb coating | | at the working | | | | | Ref |
| Pn | (Concentration | Working | dilution | | | | | Std |
| Serotype | μg/mL) | Dilution | (μg/mL) | QC1 | QC2 | QC3 | QC4 | (PC) |
| 1 | 1.185 | 15,000 | 0.09 | 0.125 | 0.031 | 0.008 | 0.00 | 0.50 |
| 3 | 0.25 | 200,000 | 0.05 | 0.125 | 0.031 | 0.008 | 0.00 | 0.50 |
| 4 | 0.25 | 50,000 | 0.18 | 0.500 | 0.125 | 0.031 | 0.00 | 2.00 |
| 5 | 0.25 | 600 | 1.67 | 0.500 | 0.125 | 0.031 | 0.00 | 2.00 |
| 6A | 1.053 | 3,000 | 1.52 | 0.625 | 0.156 | 0.039 | 0.00 | 2.50 |
| 6B | 0.50 | 1,000 | 9.58 | 0.125 | 0.031 | 0.008 | 0.00 | 0.50 |
| 7F | 1.00 | 6,500 | 1.55 | 2.500 | 0.625 | 0.156 | 0.00 | 10.00 |
| 9V | 1.00 | 10,000 | 1.03 | 0.250 | 0.063 | 0.016 | 0.00 | 1.00 |
| 14 | 0.25 | 12,000 | 0.84 | 0.500 | 0.125 | 0.031 | 0.00 | 2.00 |
| 18C | 0.25 | 1,000 | 9.97 | 0.500 | 0.125 | 0.031 | 0.00 | 2.00 |
| 19A | 0.534 | 50,000 | 0.09 | 0.125 | 0.031 | 0.008 | 0.00 | 0.50 |
| 19F | 0.134 | 400,000 | 0.01 | 2.500 | 0.625 | 0.156 | 0.00 | 10.00 |
| 22F | 0.833 | 5,000 | 0.68 | 0.500 | 0.125 | 0.031 | 0.00 | 2.00 |
| 23F | 0.533 | 75,000 | 0.01 | 5.000 | 1.250 | 0.313 | 0.00 | 20.00 |
| 33F | 0.133 | 5,000 | 0.20 | 0.500 | 0.125 | 0.031 | 0.00 | 2.00 |

Tertiary antibody working dilution 1:500
Primary incubation - 2 h ± 10 minutes @ 23 ± 2° C. with 600-800 rpm agitation
Secondary incubation - 45 minutes ± 5 minutes @ 23 ± 2° C. with 600-800 rpm agitation
Tertiary incubation - 45 minutes ± 5 minutes @ 23 ± 2° C. with 600-800 rpm agitation
mAb (monoclonal antibody);
Pn (pneumococcal);
QC (quality control);
sAB (secondary mAb);
SSUAD (serotype-specific urine antigen detection)

### Assay Qualification-Accuracy

The average recovery of 15 Vaxneuvance™ PnPs in spiked samples was within the expected range of 80-125% throughout the defined quantifiable range for 13 of the 15 STs. Throughout the defined quantifiable range, ST 9V had two spike concentrations (0.0156 and 0.0325 ng/mL) where average recoveries were 79%; the average recovery of ST 1 was outside the expected range for 3 spike concentrations (76-79%) primarily due to one sample that had very low relative accuracy estimates, weighing down the average recovery. After excluding that sample, the average recovery of ST 1 was within the expected range of 80-125% except for 78% recovery at 0.25 ng/ml spike concentration.

### Assay Qualification—Precision and Ruggedness

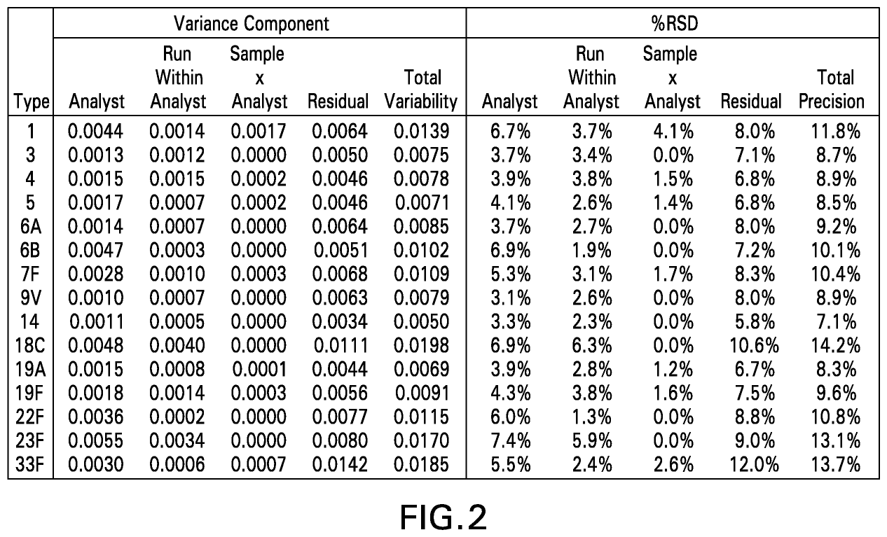

Across all Vaxneuvance™ STs, intra-assay precision estimates ranged between 4.9% and 8.5%, while total precision estimates ranged between 7.1% and 14.2% (FIG. 2). Across the 15 Vaxneuvance™ STs, all differences between the analysts were within +15% and all pairwise differences between Luminex™ instruments were within +10% (Table 2).

TABLE 2
Ruggedness (Qualification) - Mean PnPs Concentration (95%
CI) By Analyst and Luminex ™ Instrument

| | | Analyst | | Instrument | |
|---|---|---|---|---|---|
| | Factor | Concentration (ng/mL) | | Concentration (ng/mL) | |
| Type | Level | LS Mean | 95% CI | LS Mean | 95% CI |
| 1 | 1 | 0.262 | (0.257, 0.267) | 0.256 | (0.250, 0.263) |
| | 2 | 0.238 | (0.233, 0.242) | 0.243 | (0.237, 0.250) |
| | 3 | NA | NA | 0.256 | (0.249, 0.263) |
| | 4 | NA | NA | 0.243 | (0.236, 0.249) |
| 3 | 1 | 0.081 | (0.080, 0.082) | 0.079 | (0.077, 0.080) |
| | 2 | 0.076 | (0.075, 0.077) | 0.077 | (0.076, 0.079) |
| | 3 | NA | NA | 0.078 | (0.077, 0.080) |
| | 4 | NA | NA | 0.08 | (0.078, 0.082) |
| 4 | 1 | 0.332 | (0.328, 0.337) | 0.327 | (0.320, 0.333) |
| | 2 | 0.312 | (0.308, 0.316) | 0.319 | (0.313, 0.325) |
| | 3 | NA | NA | 0.317 | (0.311, 0.324) |
| | 4 | NA | NA | 0.327 | (0.321, 0.333) |
| 5 | 1 | 0.331 | (0.327, 0.336) | 0.321 | (0.315, 0.327) |
| | 2 | 0.312 | (0.308, 0.316) | 0.315 | (0.310, 0.321) |
| | 3 | NA | NA | 0.324 | (0.318, 0.330) |
| | 4 | NA | NA | 0.326 | (0.321, 0.332) |
| 6A | 1 | 0.332 | (0.328, 0.337) | 0.325 | (0.318, 0.331) |
| | 2 | 0.314 | (0.310, 0.318) | 0.318 | (0.312, 0.324) |
| | 3 | NA | NA | 0.325 | (0.318, 0.331) |
| | 4 | NA | NA | 0.325 | (0.319, 0.331) |
| 6B | 1 | 0.129 | (0.128, 0.131) | 0.124 | (0.122, 0.127) |
| | 2 | 0.118 | (0.116, 0.119) | 0.121 | (0.119, 0.123) |
| | 3 | NA | NA | 0.125 | (0.122, 0.128) |
| | 4 | NA | NA | 0.124 | (0.121, 0.126) |
| 7F | 1 | 2.567 | (2.524, 2.610) | 2.52 | (2.460, 2.580) |
| | 2 | 2.383 | (2.343, 2.423) | 2.409 | (2.354, 2.465) |
| | 3 | NA | NA | 2.501 | (2.439, 2.566) |
| | 4 | NA | NA | 2.463 | (2.407, 2.521) |
| 9V | 1 | 0.202 | (0.199, 0.204) | 0.199 | (0.195, 0.203) |
| | 2 | 0.192 | (0.190, 0.195) | 0.194 | (0.190, 0.198) |
| | 3 | NA | NA | 0.198 | (0.193, 0.202) |
| | 4 | NA | NA | 0.197 | (0.193, 0.200) |
| 14 | 1 | 0.424 | (0.419, 0.428) | 0.419 | (0.413, 0.426) |
| | 2 | 0.404 | (0.400, 0.408) | 0.406 | (0.400, 0.412) |
| | 3 | NA | NA | 0.418 | (0.411, 0.425) |
| | 4 | NA | NA | 0.411 | (0.405, 0.418) |
| 18C | 1 | 0.874 | (0.849, 0.900) | 0.845 | (0.812, 0.880) |
| | 2 | 0.787 | (0.765, 0.811) | 0.799 | (0.767, 0.833) |
| | 3 | NA | NA | 0.835 | (0.799, 0.872) |
| | 4 | NA | NA | 0.84 | (0.807, 0.874) |
| 19A | 1 | 0.117 | (0.116, 0.119) | 0.115 | (0.113, 0.117) |
| | 2 | 0.111 | (0.109, 0.112) | 0.111 | (0.109, 0.113) |
| | 3 | NA | NA | 0.116 | (0.114, 0.119) |
| | 4 | NA | NA | 0.113 | (0.111, 0.115) |
| 19F | 1 | 2.378 | (2.344, 2.412) | 2.324 | (2.278, 2.371) |
| | 2 | 2.228 | (2.196, 2.260) | 2.231 | (2.187, 2.277) |
| | 3 | NA | NA | 2.35 | (2.301, 2.402) |
| | 4 | NA | NA | 2.301 | (2.256, 2.348) |
| 22F | 1 | 0.794 | (0.779, 0.808) | 0.765 | (0.747, 0.784) |
| | 2 | 0.73 | (0.716, 0.743) | 0.748 | (0.728, 0.768) |
| | 3 | NA | NA | 0.768 | (0.748, 0.790) |
| | 4 | NA | NA | 0.762 | (0.742, 0.783) |
| 23F | 1 | 3.58 | (3.518, 3.642) | 3.473 | (3.389, 3.560) |
| | 2 | 3.205 | (3.150, 3.260) | 3.237 | (3.160, 3.316) |
| | 3 | NA | NA | 3.403 | (3.315, 3.493) |
| | 4 | NA | NA | 3.44 | (3.358, 3.524) |
| 33F | 1 | 0.835 | (0.815, 0.856) | 0.827 | (0.800, 0.855) |
| | 2 | 0.774 | (0.755, 0.794) | 0.774 | (0.747, 0.801) |
| | 3 | NA | NA | 0.842 | (0.810, 0.875) |
| | 4 | NA | NA | 0.776 | (0.750, 0.803) |

CI (confidence interval);
LS (least squares);
PnPs (pneumococcal polysaccharide)

### Assay Qualification-Sensitivity, LOD and LOQ

Except for ST 6A, LODs were below the established LLOQ for all Vaxneuvance™ STs and were consistent with results from assay development. The lower LOD for ST 6A (0.0390625 ng/mL) was greater than the development LOD (0.0097656 ng/mL). Given the change in LOD, the dilution-corrected LLOQ for ST 6A required an adjustment from 0.01953125 ng/ml to 0.078125 ng/mL. Based on results of specificity testing (see “Specificity”, below), the LLOQ for ST 19F was increased from 0.3125 ng/mL to 1.00 ng/mL. Therefore, adjusted LOQs ensured that LODs were less than the LLOQ for all Vaxneuvance STs and covered the full range of the reference standard.

### Assay Qualification—Specificity

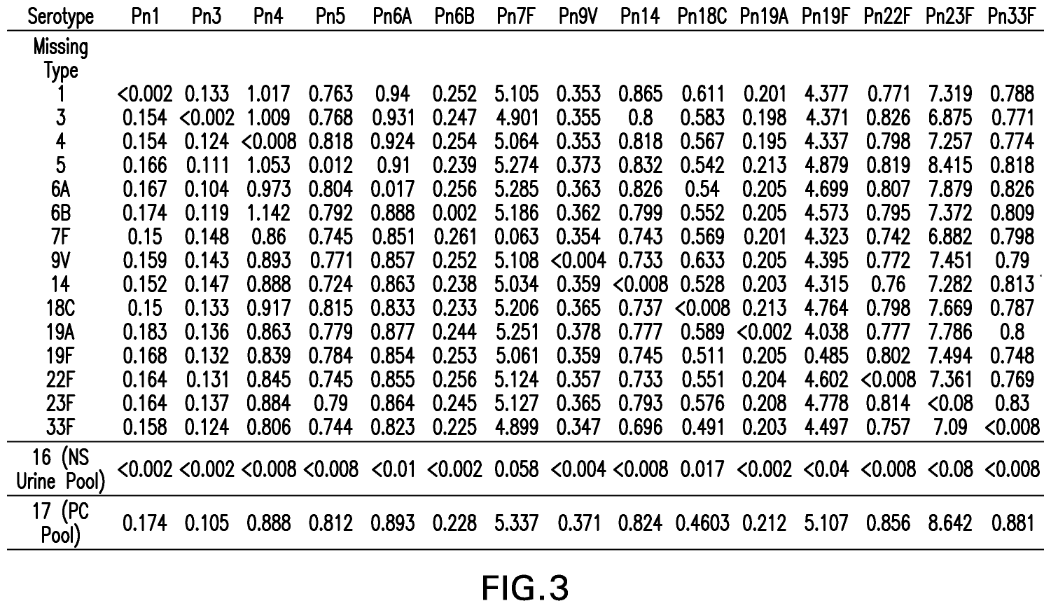

For 13 of the 15 Vaxneuvance™ STs, average measured concentrations for spiked PnPs were within two-fold of spike concentrations; however, for ST 3 and ST 18C, the measured concentrations bordered on 50% that of the nominal spike concentration, respectively (FIG. 3). For 14 of the 15 Vaxneuvance™ STs, measured concentrations for the missing PnPs were below their determined LLOQs. The average measured concentration for ST 19F was 0.485 ng/ml for the ST 19F missing sample, indicating potential interference at the current LLOQ. Based on this observation, the dilution-corrected LLOQ was adjusted from 0.3125 ng/ml to 1.00 ng/ml for ST 19F (see “Sensitivity, LOD and LOQ”, above).

### Assay Qualification—Selectivity

For 14 of the 15 Vaxneuvance™ STs, the average recovery was within the expected range of 80-125% at each pre-dilution level of urine. For ST 1, the average recovery was consistent but clustered around the upper limit of acceptability, from 118.6% in neat urine to 138.9% in urine pre-diluted 1:128.

### Assay Qualification—Dilutional Linearity

The overall dilution bias estimates per 10-fold dilution were between-15% to +15% for 14 of the 15 STs. For ST 18C, the estimated dilution bias was +74% per 10-fold dilution (data not shown).

### Assay Validation

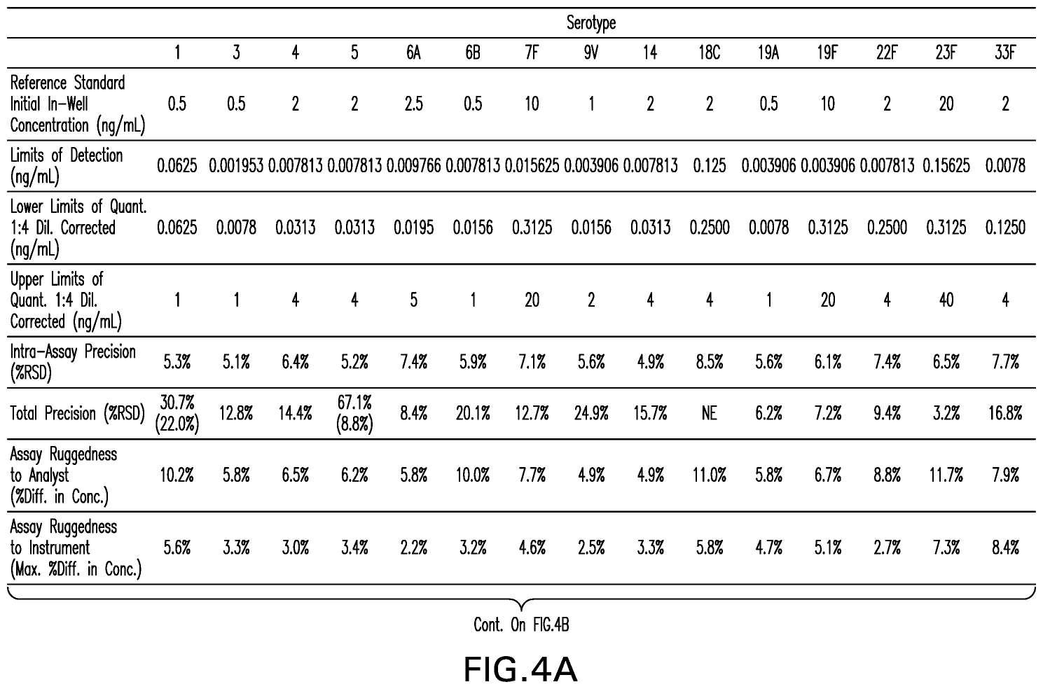

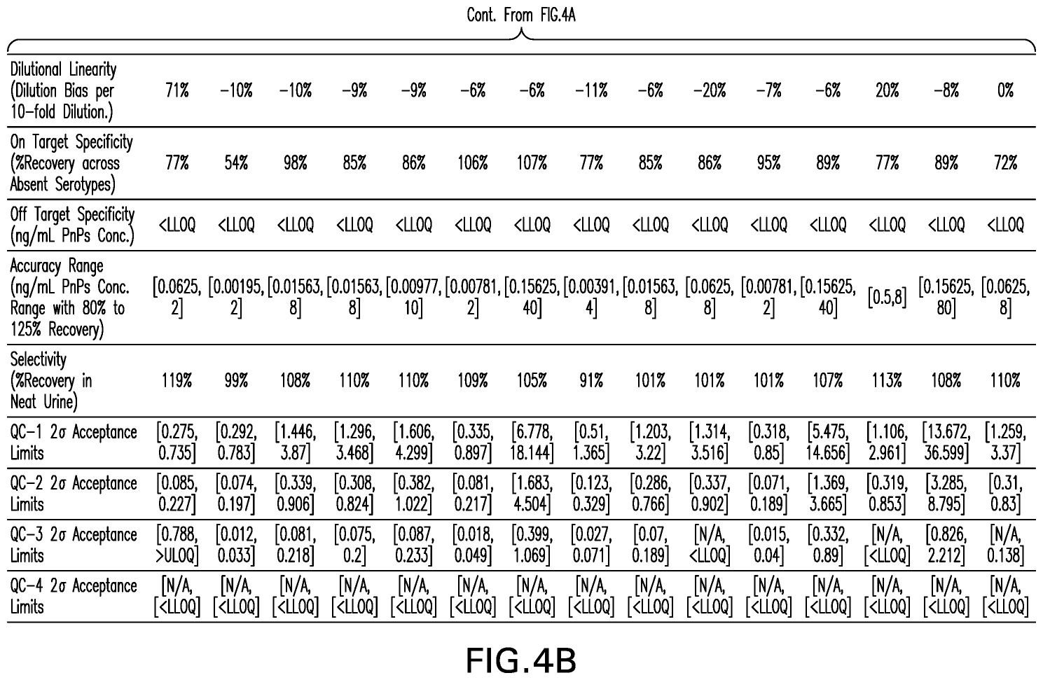

The consolidated results for all validation experiments are shown in FIG. 4.

### Assay Validation—Accuracy

During validation experiments, the concentrations of Vaxneuvance™ PnPs recovered in test samples were within 80-125% of spiked concentrations throughout the defined quantifiable range for 14 of the 15 Vaxneuvance™ STs (data not shown). Recovery of ST 19A was lower than expected (from 42-81%); however, the observed inaccuracy was not judged to be a disqualifying characteristic, given that the assay measured extremely low levels of ST 19A in urine (LLOQ of 0.0078 ng/ml).

### Assay Validation—Precision and Ruggedness

Twelve of the 14 evaluable Vaxneuvance™ STs had % CV<25%, with total assay precision estimates between 3.2% and 24.9% (Data not shown). ST18C could not be evaluated for precision due to the unavailability of 18C-positive clinical samples. ST 1 and ST 5 had relatively high % CV of 30.7% and 67.1%, respectively, driven by a single observation in each case. Upon excluding the observations with extreme concentrations, the % CVs for ST 1 and ST 5 were 22.0% and 8.8%, respectively.

Across the 14 evaluable STs and after excluding outliers noted for ST 1 and ST 5, all differences between levels of ruggedness factors (analyst, bead lot, and Luminex™ instrument) were <30% (data not shown).

### Assay Validation—Sensitivity, LOD and LOQ

The LOQs defined in the qualification study were supported by the validation study and are recommended LOQs for clinical testing. For each of the 15 Vaxneuvance™ STs, the larger of the LODs between the qualification study and the validation study was defined as the LOD for the SSUAD assay. Validation LODs were at least two-fold below the defined LLOQ for 14 of the 15 STs, while for ST 1, the LOD was equal to the recommended LLOQ (0.0625 ng/ml).

The recommended LOQs for clinical testing are conservatively set to be the LOQs defined in the qualification.

### Assay Validation—Specificity

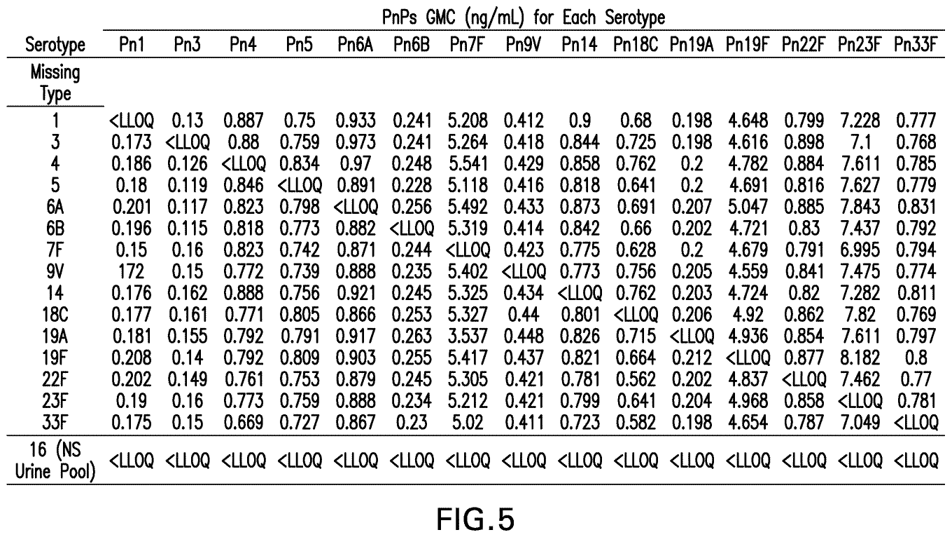

For all 15 Vaxneuvance™ STs, measured concentrations for missing STs were below their determined LLOQs (FIG. 5). Accordingly, the SSUAD assay was determined to have acceptable specificity.

### Assay Validation—Selectivity

The average recovery of known spike concentrations at each pre-dilution level of urine were within 80-125% for 14 of the 15 STs (data not shown). The average recovery for ST 19A was 71% in neat urine and 74% in 1:2-diluted urine; however, for the remaining pre-diluted samples, recovery ranged from 81-104%. Considering the SSUAD assay could measure very low levels of ST 19A (LLOQ: 0.0078 ng/mL), the magnitude of interference was not judged to be a disqualifying characteristic.

### Assay Validation—Dilutional Linearity

Overall dilution fold-bias estimates per 10-fold dilution were between-12% and +28% for 14/15 Vaxneuvance™ STs and +97% for ST 1.

### Clinical Assay Validation

A total of 129 tests were run with 34 clinical (CAPNETZ) samples positive for Vaxneuvance™ STs. Each sample was tested up to four times in separate runs, except for three samples without a sufficient volume. The SSUAD assay detected the appropriate culture-confirmed Vaxneuvance™ ST in 128 out of 129 instances.

Across the 37 CAPNETZ samples, positive results for Vaxneuvance STs other than those previously identified by positive culture were obtained in 6 samples for ST 3 (range from 0.017-139 ng/mL) and 5 samples for ST 19A (range from 0.019-0.06 ng/mL), although most were only slightly above the 2×LLOQ cut-off point of 0.016 ng/mL. The unanticipated positive results for ST 3 and ST 19A suggested that a fold-multiplier >2 would be more appropriate for these STs.

What is claimed is:

1. A method for detecting the presence or absence of a pneumococcal serotype (ST) capsular polysaccharide (PnPs) in a sample, the method comprising:
a) contacting the sample with: a first monoclonal antibody (mAb), or a functional variant thereof, that binds the pneumococcal serotype (ST) capsular polysaccharide (PnPs) in the sample to form a first mAb-antigen complex;
b) contacting the sample with: a second monoclonal antibody (mAb), or a functional variant thereof, that binds the pneumococcal serotype (ST) capsular polysaccharide (PnPs) to form a first mAb-antigen-second mAb complex;
c) contacting the sample with: a third antibody that binds the second monoclonal antibody (mAb), or a functional variant thereof, wherein the third antibody is detectably coupled to a reporter molecule;
d) detecting the presence or absence of the first mAb-antigen-second mAb complex by detecting the reporter molecule;
wherein the presence of the first mAb-antigen-second mAb complex confirms the presence of a pneumococcal serotype (ST) capsular polysaccharide (PnPs); and
wherein the absence of the first mAb-antigen-second mAb complex is indicative of the absence of a pneumococcal serotype (ST) capsular polysaccharide (PnPs).

2. The method of claim 1 wherein the first monoclonal antibody, or a functional variant thereof, specifically binds to a particular pneumococcal serotype capsular polysaccharide selected from ST-1, ST-3, ST-4, ST-5, ST-6A, ST-6B, ST-7F, ST-9V, ST-14, ST-18C, ST-19A, ST-19F, ST-22F, ST-23F and ST-33F.

3. The method of claim 1 wherein the first monoclonal antibody, or a functional variant thereof, comprises:
a) six complementarity determining regions (CDRs) selected from the group consisting of SEQ. ID. NOs.: 1-6, 21-26, 41-46, 61-66, 81-86, 101-106, 121-126, 141-146, 161-166, 181-186, 201-206, 221-226, 241-246, 261-266, and 281-286;
b) a variable heavy chain and variable light chain sequence selected from the group consisting of SEQ ID NOs: 13-14, 33-34, 53-54, 73-74, 93-94, 113-114, 133-134, 153-154, 173-174, 193-194, 213-214, 233-234, 253-254, 273-274, and 293-294; or
c) a full length light and heavy chain sequence selected from the group consisting of SEQ ID NOs: 17-18, 37-38, 57-58, 77-78, 97-98, 117-118, 137-138, 157-158, 177-178, 197-198, 217-218, 237-238, 257-258, 277-278, and 297-298.

4. The method of claim 1 wherein the second monoclonal antibody, or a functional variant thereof, specifically binds to a particular pneumococcal serotype capsular polysaccharide selected from ST-1, ST-3, ST-4, ST-5, ST-6A, ST-6B, ST-7F, ST-9V, ST-14, ST-18C, ST-19A, ST-19F, ST-22F, ST-23F and ST-33F.

5. The method of claim 1 wherein the second monoclonal antibody, or a functional variant thereof, comprises:
a) six complementarity determining regions (CDRs) selected from the group consisting of SEQ. ID. NOs.: 7-12, 27-32, 47-52, 67-72, 87-92, 107-112, 127-132, 147-152, 167-172, 187-192, 207-212, 227-232, 247-252, 267-272, and 287-292;
b) a variable heavy chain and variable light chain sequence selected from the group consisting of SEQ ID NOs: 15-16, 35-36, 55-56, 75-76, 95-96, 115-116, 135-136, 155-156, 175-176, 195-196, 215-216, 235-236, 255-256, 275-276, and 295-296; or
c) a full length light and heavy chain sequence selected from the group consisting of SEQ ID NOs: 19-20, 39-40, 59-60, 79-80, 99-100, 119-120, 139-140, 159-160, 179-180, 199-200, 219-220, 239-240, 259-260, 279-280, and 299-300.

6. The method of claim 1 further comprising contacting the sample with one or more first and second monoclonal antibodies (mAbs), to form one or more first mAb-antigen-second mAb complexes, wherein the one or more first and second monoclonal antibodies (mAbs) bind to one or more S. pneumoniae serotype-specific capsular polysaccharides.

7. The method of claim 1 wherein the first monoclonal antibody is coupled to a bead.

8. The method of claim 7 wherein the bead is made of a carboxylated polystyrene material.

9. The method of claim 7 wherein the bead is a carboxylated polystyrene microsphere.

10. The method of claim 7 wherein the bead is a magnetic Luminex™ bead.

11. The method of claim 1 wherein the sample is a human blood sample, a human saliva sample and/or a human urine sample.

12. The method of claim 11 wherein the sample is a human urine sample.

13. The method of claim 1 wherein the reporter molecule coupled to the third antibody is phycoerythrin (PE) and binds to the second monoclonal antibody to allow detection and quantitation of the captured PnPs.

14. The method of claim 1 wherein the reporter molecule coupled to the third antibody is phycoerythrin (PE) and binds to the second monoclonal antibody to allow detection and quantitation of a captured PnPs using a Luminex microfluidics system™.

15. A kit for detecting the presence or absence of one or more S. pneumoniae capsular polysaccharide(s) (PnPs) in a sample, wherein said kit comprises:
a) one or more first monoclonal antibody (mAb), or functional variant thereof, that binds one or more pneumococcal serotype (ST) capsular polysaccharide (PnPs), said antibody, or functional variant thereof, comprises:
i) six complementarity determining regions (CDRs) selected from the group consisting of SEQ. ID. NOs.: 1-6, 21-26, 41-46, 61-66, 81-86, 101-106, 121-126, 141-146, 161-166, 181-186, 201-206, 221-226, 241-246, 261-266, and 281-286;
ii) a variable heavy chain and a variable light chain having an amino acid sequence selected from the group consisting of SEQ ID NOs: 13-14, 33-34, 53-54, 73-74, 93-94, 113-114, 133-134, 153-154, 173-174, 193-194, 213-214, 233-234, 253-254, 273-274, and 293-294; or
iii) a full length light chain and a full length heavy chain having an amino acid sequence selected from the group consisting of SEQ ID NOs: 17-18, 37-38, 57-58, 77-78, 97-98, 117-118, 137-138, 157-158, 177-178, 197-198, 217-218, 237-238, 257-258, 277-278, and 297-298;
b) one or more second monoclonal antibody (mAb), or functional variant thereof, that binds one or more pneumococcal serotype (ST) capsular polysaccharide (PnPs), said antibody, or functional variant thereof, comprises:
i) six complementarity determining regions (CDRs) selected from the group consisting of SEQ. ID. NOs.: 7-12, 27-32, 47-52, 67-72, 87-92, 107-112, 127-132, 147-152, 167-172, 187-192, 207-212, 227-232, 247-252, 267-272, and 287-292;
ii) a variable heavy chain and variable light chain sequence selected from the group consisting of SEQ ID NOs: 15-16, 35-36, 55-56, 75-76, 95-96, 115-116, 135-136, 155-156, 175-176, 195-196, 215-216, 235-236, 255-256, 275-276, and 295-296; or
iii) a full length light and heavy chain sequence selected from the group consisting of SEQ ID NOs: 19-20, 39-40, 59-60, 79-80, 99-100, 119-120, 139-140, 159-160, 179-180, 199-200, 219-220, 239-240, 259-260, 279-280, and 299-300;
c) a third antibody that binds the second monoclonal antibody; and
d) instructions to use said kit.

16. A monoclonal antibody (mAb), or a functional variant thereof, that specifically binds to a pneumococcal serotype (ST) capsular polysaccharide (PnPs), wherein said mAb comprises:
a) six complementarity determining regions (CDRs) selected from the group consisting of SEQ. ID. NOs.: 1-6, 21-26, 41-46, 61-66, 81-86, 101-106, 121-126, 141-146, 161-166, 181-186, 201-206, 221-226, 241-246, 261-266, 281-286, 7-12, 27-32, 47-52, 67-72, 87-92, 107-112, 127-132, 147-152, 167-172, 187-192, 207-212, 227-232, 247-252, 267-272, and 287-292;
b) a variable heavy chain and a variable light chain having an amino acid sequence selected from the group consisting of SEQ ID NOs: 13-14, 33-34, 53-54, 73-74, 93-94, 113-114, 133-134, 153-154, 173-174, 193-194, 213-214, 233-234, 253-254, 273-274, 293-294, 15-16, 35-36, 55-56, 75-76, 95-96, 115-116, 135-136, 155-156, 175-176, 195-196, 215-216, 235-236, 255-256, 275-276, and 295-296; or
c) a full length light chain and a full length heavy chain having an amino acid sequence selected from the group consisting of SEQ ID NOs: 17-18, 37-38, 57-58, 77-78, 97-98, 117-118, 137-138, 157-158, 177-178, 197-198, 217-218, 237-238, 257-258, 277-278, 297-298, 19-20, 39-40, 59-60, 79-80, 99-100, 119-120, 139-140, 159-160, 179-180, 199-200, 219-220, 239-240, 259-260, 279-280, and 299-300.

17. The monoclonal antibody of claim 16, wherein the mAb comprises six CDRs selected from the group consisting of SEQ. ID. NOs.: 1-6, 21-26, 41-46, 61-66, 81-86, 101-106, 121-126, 141-146, 161-166, 181-186, 201-206, 221-226, 241-246, 261-266, and 281-286.

18. The monoclonal antibody of claim 16, wherein the mAb comprises six CDRs selected from the group consisting of SEQ. ID. NOs.: 7-12, 27-32, 47-52, 67-72, 87-92, 107-112, 127-132, 147-152, 167-172, 187-192, 207-212, 227-232, 247-252, 267-272, and 287-292.

19. The monoclonal antibody of claim 16, wherein the mAb comprises a variable heavy chain and a variable light chain having an amino acid sequence selected from the group consisting of SEQ ID NOs: 13-14, 33-34, 53-54, 73-74, 93-94, 113-114, 133-134, 153-154, 173-174, 193-194, 213-214, 233-234, 253-254, 273-274, and 293-294.

20. The monoclonal antibody of claim 16, wherein the mAb comprises a variable heavy chain and a variable light chain having an amino acid sequence selected from the group consisting of SEQ ID NOs: 15-16, 35-36, 55-56, 75-76, 95-96, 115-116, 135-136, 155-156, 175-176, 195-196, 215-216, 235-236, 255-256, 275-276, and 295-296.

21. The monoclonal antibody of claim 16, wherein the mAb comprises a full length light chain and a full length heavy chain having an amino acid sequence selected from the group consisting of SEQ ID NOs: 17-18, 37-38, 57-58, 77-78, 97-98, 117-118, 137-138, 157-158, 177-178, 197-198, 217-218, 237-238, 257-258, 277-278, and 297-298.

22. The monoclonal antibody of claim 16, wherein the mAb comprises a full length light chain and a full length heavy chain having an amino acid sequence selected from the group consisting of SEQ ID NOs: 19-20, 39-40, 59-60, 79-80, 99-100, 119-120, 139-140, 159-160, 179-180, 199-200, 219-220, 239-240, 259-260, 279-280, and 299-300.
